(19) United States
(12) Patent Application Publication
(10) Pub. No.: US 2023/0348628 A1
(43) Pub. Date: Nov. 2, 2023

(54) PHARMACEUTICAL COMPOSITIONS OF ANTI-CD20/ANTI-CD3 BISPECIFIC ANTIBODIES AND METHODS OF USE

(71) Applicant: Hoffmann-La Roche Inc., Little Falls, NJ (US)

(72) Inventors: Ellen Dorothee MEUX, Grenzach-Wyhlen (DE); Satya Krishna Kishore RAVURI, Pfeffingen (CH); Karin SCHOENHAMMER, Weil am Rhein (DE); Ilona Elisabeth VOLLRATH, Steinen (DE); Jérémy Jean-Pierre DUBOEUF, Hegenheim (FR)

(21) Appl. No.: 18/299,877

(22) Filed: Apr. 13, 2023

Related U.S. Application Data
(60) Provisional application No. 63/330,748, filed on Apr. 13, 2022.

(51) Int. Cl. C07K 16/46; C07K 16/28; A61K 9/08; A61K 47/18; A61K 47/36; A61K 47/26

(52) U.S. Cl. CPC C07K 16/468; C07K 16/2887; C07K 16/2809; A61K 9/08; A61K 47/183; A61K 47/36; A61K 47/26; A61K 2039/505

(57) ABSTRACT

The present invention relates to pharmaceutical compositions of anti-CD20/anti-CD3 bispecific antibodies and methods of using the same.

### SEQUENCE LISTING

The instant application contains a Sequence Listing which has been submitted electronically in XML format and is hereby incorporated by reference in its entirety. Said XML copy, created on Apr. 6, 2023, is named 51177-044002_Sequence_Listing_4_6_23 and is 53,430 bytes in size.

### FIELD OF THE INVENTION

The present invention relates to pharmaceutical compositions of anti-CD20/anti-CD3 bispecific antibodies and methods of using the same.

### BACKGROUND OF THE INVENTION

One of the major challenges in the development of biotech therapeutics is protein stability, which has to be maintained during multiple process steps involved on their way to market. Furthermore, protein stability has to be maintained during storage as well as during administration to the patient. Therapeutic antibodies can be formulated in an aqueous carrier for administration to a subject, e.g., by intravenous or subcutaneous administration. During storage, handling, and administration of such pharmaceutical compositions, it is necessary to mitigate loss of the therapeutic antibody, which can occur through degradation and surface adsorption, such as protein adsorption to surfaces of filters, storage canisters, tubing, syringes, intravenous fluid bags, and other containers. Both low- and high-concentration formulations pose their own challenges during research and development as well as manufacturing. For example, low concentrations are highly affected by surface adsorption whereas high concentrations can show high viscosities.

In instances in which the pharmaceutical composition contains a relatively low concentration of therapeutic protein, protein loss can be dramatically increased by these factors, resulting reduction in reduced therapeutic efficacy of the pharmaceutical composition

Thus, there is a need in the field to develop pharmaceutical formulations in which an anti-CD20/anti-CD3 bispecific antibody (e.g., low-dose anti-CD20/anti-CD3 bispecific antibody, e.g., low-dose anti-CD20/anti-CD3 T cell-engaging bispecific antibody, e.g., glofitamab) is stable and protected from loss due to adsorption.

### SUMMARY OF THE INVENTION

The present invention relates to pharmaceutical compositions of anti-CD20/anti-CD3 bispecific antibodies (e.g., anti-CD20/anti-CD3 T cell-engaging bispecific antibodies (TCB), e.g., glofitamab, RO7082859, or RG6026) and methods of using the same. The disclosed compositions and related methods address the problem of delivering anti-CD20/anti-CD3 bispecific antibodies (e.g., anti-CD20/anti-CD3 TCB, e.g., glofitamab) that are formulated at low concentration, ensuring that patients receive the intended dose of the anti-CD20/anti-CD3 bispecific antibody (e.g., anti-CD20/anti-CD3 TCB, e.g., glofitamab) with little to no loss of the protein during storage and administration.

In one aspect, the invention features a liquid pharmaceutical composition comprising:
about 1 to 25 mg/ml of an anti-CD20/anti-CD3 bispecific antibody;
about 10 to 50 mM of a buffering agent;
about ≥200 mM of a tonicity agent;
about 0-15 mM methionine; and
about ≥0.2 mg/ml of a surfactant;
at a pH in the range of from about 5.0 to about 6.0,
wherein the anti-CD20/anti-CD3 bispecific antibody comprises
a) at least one antigen binding domain that specifically binds to CD20 comprising a heavy chain variable region comprising:
   (i) an HVR-H1 comprising the amino acid sequence of SEQ ID NO: 1;
   (ii) an HVR-H2 comprising the amino acid sequence of SEQ ID NO: 2; and
   (iii) an HVR-H3 comprising the amino acid sequence of SEQ ID NO: 3;
and a light chain variable region comprising:
   (i) an HVR-L1 comprising the amino acid sequence of SEQ ID NO: 4;
   (ii) an HVR-L2 comprising the amino acid sequence of SEQ ID NO: 5; and
   (iii) an HVR-L3 comprising the amino acid sequence of SEQ ID NO: 6; and
b) at least one antigen binding domain that specifically binds to CD3 comprising a heavy chain variable region comprising:
   (i) an HVR-H1 comprising the amino acid sequence of SEQ ID NO: 9;
   (ii) an HVR-H2 comprising the amino acid sequence of SEQ ID NO: 10; and
   (iii) an HVR-H3 comprising the amino acid sequence of SEQ ID NO: 11; and
a light chain variable region comprising:
   (i) an HVR-L1 comprising the amino acid sequence of SEQ ID NO: 12;
   (ii) an HVR-L2 comprising the amino acid sequence of SEQ ID NO: 13; and
   (iii) an HVR-L3 comprising the amino acid sequence of SEQ ID NO: 14.

In one embodiment, the anti-CD20/anti-CD3 bispecific antibody (e.g., anti-CD20/anti-CD3 TCB, e.g., glofitamab) concentration is in the range of about 1 to 5 mg/ml. In one embodiment, the anti-CD20/anti-CD3 bispecific antibody (e.g., anti-CD20/anti-CD3 TCB, e.g., glofitamab) concentration is in the range of about 0.9-1.1 mg/ml. In one embodiment, the anti-CD20/anti-CD3 bispecific antibody (e.g., anti-CD20/anti-CD3 TCB, e.g., glofitamab) concentration is about 1 mg/ml.

In one embodiment, the anti-CD20/anti-CD3 bispecific antibody (e.g., anti-CD20/anti-CD3 TCB, e.g., glofitamab) comprises
a) at least one antigen binding domain that specifically binds to CD20 comprising the heavy chain variable region sequence of SEQ ID NO: 7 and the light chain variable region sequence of SEQ ID NO: 8, and
b) at least one antigen binding domain that specifically binds to CD3 comprising the heavy chain variable region sequence of SEQ ID NO: 15 and the light chain variable region sequence of SEQ ID NO: 16.

In one embodiment, the anti-CD20/anti-CD3 bispecific antibody (e.g., anti-CD20/anti-CD3 TCB, e.g., glofitamab) comprises
a) a first Fab molecule which specifically binds to CD3, particularly CD3 epsilon; and wherein the variable domains VL and VH of the Fab light chain and the Fab heavy chain are replaced by each other;
b) a second Fab and a third Fab molecule which specifically bind to CD20, wherein in the constant domain CL of the second Fab and third Fab molecule the amino acid at position 124 is substituted by lysine (K) (numbering according to Kabat) and the amino acid at position 123 is substituted by lysine (K) or arginine (R), particularly by arginine (R) (numbering according to Kabat), and wherein in the constant domain CH1 o of the second Fab and third Fab molecule the amino acid at position 147 is substituted by glutamic acid (E) (EU numbering) and the amino acid at position 213 is substituted by glutamic acid (E) (EU numbering); and
c) a Fc domain composed of a first and a second subunit capable of stable association.

In one embodiment, the anti-CD20/anti-CD3 bispecific antibody is glofitamab.

In one embodiment, the buffering agent is a histidine buffer, optionally a histidine HCl buffer. In one embodiment, the buffering agent is at a concentration of about 15 to 25 mM. In one embodiment, the buffering agent is at a concentration of about 20 mM. In one embodiment, the buffering agent provides a pH of about 5.2 to about 5.8.

In one embodiment, the tonicity agent is selected from the group of salts, sugars, and amino acids. In one embodiment, the tonicity agent is either sucrose or sodium chloride. In one embodiment, the tonicity agent is sucrose at a concentration of about 200 mM or higher. In one embodiment, the tonicity agent is sucrose at a concentration of about 200 mM-280 mM. In one embodiment, the tonicity agent is sucrose at a concentration of about 240 mM.

In one embodiment, the methionine is at a concentration of about 5-15 mM.

In one embodiment, the methionine is at a concentration of about 10 mM. In one embodiment, the surfactant is at a concentration of about 0.2-0.8 mg/ml. In one embodiment, the surfactant is polysorbate or poloxamer 188. In one embodiment, the surfactant is polysorbate 20 at a concentration of 0.2-0.8 mg/ml. In one embodiment, the surfactant is polysorbate 20 at a concentration of about 0.5 mg/ml

In one embodiment, the liquid pharmaceutical composition comprises:
about 1 to 5 mg/ml of an anti-CD20/anti-CD3 bispecific antibody (e.g., anti-CD20/anti-CD3 TCB, e.g., glofitamab) comprising:
a) at least one antigen binding domain that specifically binds to CD20
   comprising a heavy chain variable region comprising:
      (i) an HVR-H1 comprising the amino acid sequence of SEQ ID NO: 1;
      (ii) an HVR-H2 comprising the amino acid sequence of SEQ ID NO: 2; and
      (iii) an HVR-H3 comprising the amino acid sequence of SEQ ID NO: 3;
   and a light chain variable region comprising:
      (i) an HVR-L1 comprising the amino acid sequence of SEQ ID NO: 4;
      (ii) an HVR-L2 comprising the amino acid sequence of SEQ ID NO: 5; and
      (iii) an HVR-L3 comprising the amino acid sequence of SEQ ID NO: 6; and
b) at least one antigen binding domain that specifically binds to CD3
   comprising a heavy chain variable region comprising:
      (i) an HVR-H1 comprising the amino acid sequence of SEQ ID NO: 9;
      (ii) an HVR-H2 comprising the amino acid sequence of SEQ ID NO: 10; and
      (iii) an HVR-H3 comprising the amino acid sequence of SEQ ID NO: 11; and
   a light chain variable region comprising:
      (i) an HVR-L1 comprising the amino acid sequence of SEQ ID NO: 12;
      (ii) an HVR-L2 comprising the amino acid sequence of SEQ ID NO: 13; and
      (iii) an HVR-L3 comprising the amino acid sequence of SEQ ID NO: 14;
about 15-25 mM of a histidine buffer;
about 200-280 mM sucrose;
about 0-15 mM methionine; and
about 0.2-0.8 mg/ml of PS20
at a pH of about 5 to about 6.

In one embodiment, the liquid pharmaceutical composition comprises:
about 1 mg/ml of glofitamab;
about 20 mM of a histidine buffer;
about 240 mM sucrose;
about 10 mM methionine; and
about 0.5 mg/ml of PS20
at a pH of about 5.5.

In one embodiment the invention provides the use of a liquid pharmaceutical composition of any of the preceding aspects and embodiments for the preparation of a medicament useful for treating a cell proliferative disorder.

In another aspect, the invention features a pharmaceutical composition of any of the preceding aspects and embodiments for use in treating or delaying progression of a cell proliferative disorder in a subject in need thereof.

In another aspect, the invention features a pharmaceutical composition of any of the preceding aspects and embodiments for use in a treating or delaying progression of a cell proliferative disorder in a subject in need thereof, comprising administering to the subject an effective amount of the pharmaceutical composition of any of the preceding aspects and embodiments.

In particular embodiments, the cell proliferative disorder is cancer.

A further aspect of the present invention relates to the invention as described herein.

Each and every embodiment can be combined unless the context clearly suggests otherwise. Each and every embodiment can be applied to each and every aspect of the invention unless the context clearly suggests otherwise.

Specific embodiments of the present invention will become evident from the following more detailed description of certain preferred embodiments and the claims.

### BRIEF DESCRIPTION OF THE DRAWINGS

FIGS. 1A-FIG. 1N: Schematic diagrams showing configurations of exemplary anti-CD20/anti-CD3 bispecific antibodies.

FIG. 2: Schematic diagram showing the structure of glofitamab.

FIG. 3: Formulation Development GLP Tox and Entry into Human Study. Surfactant content of formulations F1 to F5, initial vs. after 6 weeks of storage at 5, 25, or 40° C.

FIG. 4A-FIG. 4C: Formulation Development GLP Tox and Entry into Human Study, size exclusion chromatography (SEC) of formulations F1 to F5, initial vs. after 6 weeks of storage at 5, 25, or 40° C. FIG. 4A: Main Peak, FIG. 4B: high molecular weight (HMW); FIG. 4C. low molecular weight (LMW).

FIG. 5A-FIG. 5C: Formulation Development GLP Tox and Entry into Human Study, ion exchange chromatography (IEC) of formulations F1 to F5, initial vs. after 6 weeks of storage at 5, 25, or 40° C. FIG. 5A: Main Peak, FIG. 5B. HMW; FIG. 5C. LMW.

FIG. 6: Formulation Development—analytical results of formulation F1 up to 84 weeks. F1=5 mg/ml RO7022859 (i.e., glofitamab), 20 mM Histidine HCl pH 5.5, 240 mM Sucrose, 10 mM Methionine, 0.05% (w/v) Polysorbate 20.

FIG. 7A-FIG. 7B: Formulation Development GLP Tox and Entry into Human Study, huCD20 binding of formulations F1 to F5, initial vs. after 3 and 6 weeks of storage at 5, 25, or 40° C. (FIG. 7A) and huCD3 binding of formulations F1 to F5, initial vs. after 3 and 6 weeks of storage at 5, 25, or 40° C. (FIG. 7B.

FIG. 8A-FIG. 8B: Development Studies for Phase III and commercial formulation. Glofitamab size exclusion (SE)-HPLC % HMWS (FIG. 8A) and ion exchange (IE)-HPLC % Acidic Region (FIG. 8B) as a Function of Protein Concentration after 104 Weeks Storage at 5° C.

FIG. 9A-FIG. 9B: Development Studies for Phase III and commercial formulation. Glofitamab SE-HPLC % HMWS (FIG. 9A) and % Acidic Region (FIG. 9B) as Function of pH and Stabilizer (Methionine) Addition after 6w Storage at 40° C.

FIG. 10A-FIG. 10B: Development Studies for Phase III and commercial formulation. Glofitamab SE-HPLC % HMWS including Visible Particle Formation (FIG. 10A) and IE-HPLC % Acidic Region (FIG. 10B) as Function of Tonicity Agent after 26 Weeks Storage at 25° C.

FIG. 11A-FIG. 11B: Development Studies for Phase III and commercial formulation. Glofitamab SE-HPLC % HMWS including Visible Particle Formation (FIG. 11A) and IE-HPLC % Acidic Region (FIG. 11B) as Function of Surfactant after 7 Days of Shaking at 25° C.

FIG. 12: Development Studies for Phase III and commercial formulation. Glofitamab PS20 Content [mg/ml] and Visible Particle Formation as Function of Protein Concentration Initially and after 104 Weeks of Storage at 5° C.

FIG. 13: Long-term stability data: PS20 Content of Example Glofitamab DP Batches on Stability (Storage at 2-8° C.).

### DETAILED DESCRIPTION OF THE INVENTION

The present invention relates to pharmaceutical compositions of anti-CD20/anti-CD3 bispecific antibodies and methods of using the same. The disclosed compositions and related methods address the problem of delivering anti-CD20/anti-CD3 bispecific antibodies that are formulated at low concentration, ensuring that patients receive the intended dose of the anti-CD20/anti-CD3 bispecific antibody with little to no loss of the bispecific antibody during storage and administration.

### I. General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

### II. Definitions

Terms are used herein as generally used in the art, unless otherwise defined in the following.

The term “cluster of differentiation 20” or “CD20” as used herein, refers to any native CD20 from any vertebrate source, including mammals such as primates (e.g., humans) and rodents (e.g., mice and rats), unless otherwise indicated. CD20 (also known as B-lymphocyte antigen CD20, B-lymphocyte surface antigen B1, Leu-16, Bp35, BM5, and LF5; the human protein is characterized in UniProt database entry P11836) is a hydrophobic transmembrane protein with a molecular weight of approximately 35 kD expressed on pre-B and mature B lymphocytes (Valentine, M. A. et al., J. Biol. Chem. 264 (1989) 11282-11287; Tedder, T. F., et al., Proc. Natl. Acad. Sci. U.S.A. 85 (1988) 208-212; Stamenkovic, I., et al., J. Exp. Med. 167 (1988) 1975-1980; Einfeld, D. A., et al., EMBO J. 7 (1988) 711-717; Tedder, T. F., et al., J. Immunol. 142 (1989) 2560-2568). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. The term encompasses “full-length,” unprocessed CD20 as well as any form of CD20 that results from processing in the cell. The term also encompasses naturally occurring variants of CD20, e.g., splice variants or allelic variants. Alternative splicing of this gene results in two transcript variants which encode the same protein. In one embodiment, CD20 is human CD20.

The terms “anti-CD20 antibody” and “an antibody that binds to CD20” refer to an antibody that is capable of binding CD20 with sufficient affinity such that the antibody is useful as a diagnostic and/or therapeutic agent in targeting CD20. In one embodiment, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD20 antibody binds to an epitope of CD20 that is conserved among CD20 from different species.

By “Type II anti-CD20 antibody” is meant an anti-CD20 antibody having binding properties and biological activities of Type II anti-CD20 antibodies as described in Cragg et al., Blood 103 (2004) 2738-2743; Cragg et al., Blood 101 (2003) 1045-1052, Klein et al., mAbs 5 (2013), 22-33, and summarized in Table 1 below.

TABLE 1
Properties of type I and type II anti-CD20 antibodies

| type I anti-CD20 antibodies | type II anti-CD20 antibodies |
|---|---|
| Bind class I CD20 epitope | Bind class II CD20 epitope |
| Localize CD20 to lipid rafts | Do not localize CD20 to lipid rafts |
| High CDC * | Low CDC * |
| ADCC activity * | ADCC activity * |
| Full binding capacity to B cells | Approx. half binding capacity to B cells |
| Weak homotypic aggregation | Homotypic aggregation |
| Low cell death induction | Strong cell death induction |

* if IgG₁ isotype

Examples of type II anti-CD20 antibodies include, e.g., obinutuzumab (GA101), tositumumab (B1), humanized B-Ly1 antibody IgG1 (a chimeric humanized IgG1 antibody as disclosed in WO 2005/044859), 11B8 IgG1 (as disclosed in WO 2004/035607) and AT80 IgG1.

Examples of type I anti-CD20 antibodies include, e.g., rituximab, ofatumumab, veltuzumab, ocaratuzumab, ocrelizumab, PRO131921, ublituximab, H147 IgG3 (ECACC, hybridoma), 2C6 IgG1 (as disclosed in WO 2005/103081), 2F2 IgG1 (as disclosed in WO 2004/035607 and WO 2005/103081) and 2H7 IgG1 (as disclosed in WO 2004/056312).

“CD3” refers to any native CD3 from any vertebrate source, including mammals such as primates (e.g., humans), non-human primates (e.g., cynomolgus monkeys) and rodents (e.g., mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one embodiment, CD3 is human CD3, particularly the epsilon subunit of human CD3 (CD3ε). The amino acid sequence of human CD3ε is shown in UniProt (www.uniprot.org) accession no. P07766 (version 144), or NCBI (www.ncbi.nlm.nih.gov/) RefSeq NP_000724.1. The amino acid sequence of cynomolgus monkey [Macaca fascicularis] CD3ε is shown in NCBI GenBank no. BAB71849.1.

The terms “anti-CD20/anti-CD3 antibody,” “anti-CD20/anti-CD3 bispecific antibody,” and “a bispecific antibody that binds to CD20 and CD3” refer to a bispecific antibody that is capable of binding both CD20 and CD3 with sufficient affinity such that the antibody is useful as a diagnostic and/or therapeutic agent in targeting CD20 and/or CD3. In one embodiment, the extent of binding of a bispecific antibody that binds to CD20 and CD3 to an unrelated, non-CD3 protein and/or non-CD20 protein is less than about 10% of the binding of the antibody to CD3 and/or CD20 as measured, e.g., by a radioimmunoassay (RIA). In certain embodiments, the anti-CD20/anti-CD3 bispecific antibody binds to each of CD20 and/or CD3 with a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, a bispecific antibody that binds to CD20 and CD3 binds to an epitope of CD3 that is conserved among CD3 from different species and/or an epitope of CD20 that is conserved among CD20 from different species. One example of an anti-CD20/anti-CD3 bispecific antibody is glofitamab (WHO Drug Information (International Nonproprietary Names for Pharmaceutical Substances), Recommended INN: List 83, 2020, vol. 34, no. 1, p. 39, also known as anti-CD20/anti-CD3 T cell-engaging bispecific antibody (TCB), CD20-TCB, RO7082859, or RG6026; CAS #: 2229047-91-8).

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor. Amino acid sequence deletions and insertions include amino- and/or carboxy-terminal deletions and insertions of amino acids. Particular amino acid mutations are amino acid substitutions. For the purpose of altering, e.g., the binding characteristics of an Fc region, non-conservative amino acid substitutions, i.e., replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g., 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc region to glycine can be indicated as 329G, G329, G₃₂₉, P329G, or Pro329Gly.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., receptor and a ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art. A particular method for measuring affinity is Surface Plasmon Resonance (SPR).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

As used herein, the term “antigen binding moiety” refers to a polypeptide molecule that specifically binds to an antigenic determinant. In one embodiment, an antigen binding moiety is able to direct the entity to which it is attached (e.g., a cytokine or a second antigen binding moiety) to a target site, for example to a specific type of tumor cell or tumor stroma bearing the antigenic determinant. Antigen binding moieties include antibodies and fragments thereof as further defined herein. Preferred antigen binding moieties include an antigen binding domain of an antibody, comprising an antibody heavy chain variable region and an antibody light chain variable region. In certain embodiments, the antigen binding moieties may include antibody constant regions as further defined herein and known in the art. Useful heavy chain constant regions include any of the five isotypes: α, δ, ε, γ, or μ. Useful light chain constant regions include any of the two isotypes: κ and λ.

By “binds,” “specifically binds,” or is “specific for” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g., surface plasmon resonance technique (analyzed on a BIACORE® instrument) (Liljeblad et al., Glyco J. 17, 323-329 (2000)), and traditional binding assays (Heeley, Endocr Res. 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

“Reduced binding,” for example reduced binding to an Fc receptor, refers to a decrease in affinity for the respective interaction, as measured for example by SPR. For clarity the term includes also reduction of the affinity to zero (or below the detection limit of the analytic method), i.e., complete abolishment of the interaction. Conversely, “increased binding” refers to an increase in binding affinity for the respective interaction.

As used herein, the term “antigen binding molecule” refers in its broadest sense to a molecule that specifically binds an antigenic determinant. Examples of antigen binding molecules are immunoglobulins and derivatives, e.g., fragments, thereof.

As used herein, the term “antigenic determinant” is synonymous with “antigen” and “epitope,” and refers to a site (e.g., a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, free in blood serum, and/or in the extracellular matrix (ECM). The proteins referred to as antigens herein (e.g., CD3) can be any native form the proteins from any vertebrate source, including mammals such as primates (e.g., humans) and rodents (e.g., mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g., splice variants or allelic variants. An exemplary human protein useful as antigen is CD3, particularly the epsilon subunit of CD3 (see UniProt no. P07766 (version 130), NCBI RefSeq no. NP_000724.1, for the human sequence; or UniProt no. Q95L15 (version 49), NCBI GenBank no. BAB71849.1, for the cynomolgus [Macaca fascicularis] sequence). In certain embodiments a T cell activating bispecific antigen binding molecule described herein binds to an epitope of CD3 or a target cell antigen that is conserved among the CD3 or target cell antigen from different species.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN® (DNASTAR®) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX® operating system, including digital UNIX® V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen binding activity.

The terms “full length antibody,” “intact antibody,” and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g., scFv), and multispecific antibodies formed from antibody fragments. The term “antibody fragment” as used herein also encompasses single-domain antibodies.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five classes, called α (IgA), δ (IgD), ε (IgE), γ (IgG), or μ (IgM), some of which may be further divided into subclasses, e.g., γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Preferably, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen binding specificity.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term IgG “isotype” or “subclass” as used herein is meant any of the subclasses of immunoglobulins defined by the chemical and antigenic characteristics of their constant regions.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, EU numbering). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991 (see also above). A “subunit” of an Fc domain as used herein refers to one of the two polypeptides forming the dimeric Fc domain, i.e., a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e., the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g., antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89).

The term “effector functions” when used in reference to antibodies refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

As used herein, the term “effector cells” refers to a population of lymphocytes that display effector moiety receptors, e.g., cytokine receptors, and/or Fc receptors on their surface through which they bind an effector moiety, e.g., a cytokine, and/or an Fc region of an antibody and contribute to the destruction of target cells, e.g., tumor cells. Effector cells may for example mediate cytotoxic or phagocytic effects. Effector cells include, but are not limited to, effector T cells such as CD8⁺ cytotoxic T cells, CD4⁺ helper T cells, γδ T cells, NK cells, lymphokine-activated killer (LAK) cells, and macrophages/monocytes.

As used herein, the terms “engineer,” “engineered,” and “engineering,” are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. “Engineering”, particularly with the prefix “glyco-”, as well as the term “glycosylation engineering,” includes metabolic engineering of the glycosylation machinery of a cell, including genetic manipulations of the oligosaccharide synthesis pathways to achieve altered glycosylation of glycoproteins expressed in cells. Furthermore, glycosylation engineering includes the effects of mutations and cell environment on glycosylation. In one embodiment, the glycosylation engineering is an alteration in glycosyltransferase activity. In a particular embodiment, the engineering results in altered glucosaminyltransferase activity and/or fucosyltransferase activity. Glycosylation engineering can be used to obtain a “host cell having increased GnTIII activity” (e.g., a host cell that has been manipulated to express increased levels of one or more polypeptides having β(1,4)-N-acetylglucosaminyltransferase III (GnTIII) activity), a “host cell having increased ManII activity” (e.g., a host cell that has been manipulated to express increased levels of one or more polypeptides having α-mannosidase II (ManII) activity), or a “host cell having decreased α(1,6) fucosyltransferase activity” (e.g., a host cell that has been manipulated to express decreased levels of α(1,6) fucosyltransferase).

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate proteins used for the present invention. In one embodiment, the host cell is engineered to allow the production of an antibody with modified oligosaccharides. In certain embodiments, the host cells have been manipulated to express increased levels of one or more polypeptides having β(1,4)-N-acetylglucosaminyltransferase III (GnTIII) activity. In certain embodiments the host cells have been further manipulated to express increased levels of one or more polypeptides having α-mannosidase II (ManII) activity. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

As used herein, the term “polypeptide having GnTIII activity” refers to a polypeptide that is able to catalyze the addition of a N-acetylglucosamine (GlcNAc) residue in β-1,4 linkage to the β-linked mannoside of the trimannosyl core of N-linked oligosaccharides. This includes fusion polypeptides exhibiting enzymatic activity similar to, but not necessarily identical to, an activity of β(1,4)-N-acetylglucosaminyltransferase III, also known as β-1,4-mannosyl-glycoprotein 4-beta-N-acetylglucosaminyl-transferase (EC 2.4.1.144), according to the Nomenclature Committee of the International Union of Biochemistry and Molecular Biology (NC-IUBMB), as measured in a particular biological assay, with or without dose dependency. In the case where dose dependency does exist, it need not be identical to that of GnTIII, but rather substantially similar to the dose-dependency in a given activity as compared to the GnTIII (i.e., the candidate polypeptide will exhibit greater activity or not more than about 25-fold less and, preferably, not more than about ten-fold less activity, and most preferably, not more than about three-fold less activity relative to the GnTIII). In certain embodiments the polypeptide having GnTIII activity is a fusion polypeptide comprising the catalytic domain of GnTIII and the Golgi localization domain of a heterologous Golgi resident polypeptide. Particularly, the Golgi localization domain is the localization domain of mannosidase II or GnTI, most particularly the localization domain of mannosidase II. Alternatively, the Golgi localization domain is selected from the group consisting of: the localization domain of mannosidase I, the localization domain of GnTII, and the localization domain of α1,6 core fucosyltransferase. Methods for generating such fusion polypeptides and using them to produce antibodies with increased effector functions are disclosed in WO2004/065540, U.S. Provisional Pat. Appl. No. 60/495,142 and U.S. Pat. Appl. Publ. No. 2004/0241817, the entire contents of which are expressly incorporated herein by reference.

As used herein, the term “Golgi localization domain” refers to the amino acid sequence of a Golgi resident polypeptide which is responsible for anchoring the polypeptide to a location within the Golgi complex. Generally, localization domains comprise amino terminal “tails” of an enzyme.

As used herein, the term “polypeptide having ManII activity” refers to polypeptides that are able to catalyze the hydrolysis of the terminal 1,3- and 1,6-linked α-D-mannose residues in the branched GlcNAcMan₅GlcNAc₂ mannose intermediate of N-linked oligosaccharides. This includes polypeptides exhibiting enzymatic activity similar to, but not necessarily identical to, an activity of Golgi α-mannosidase II, also known as mannosyl oligosaccharide 1,3-1,6-α-mannosidase II (EC 3.2.1.114), according to the Nomenclature Committee of the International Union of Biochemistry and Molecular Biology (NC-IUBMB).

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

By “antibody having increased/reduced antibody dependent cell-mediated cytotoxicity (ADCC)” is meant an antibody having increased/reduced ADCC as determined by any suitable method known to those of ordinary skill in the art. One accepted in vitro ADCC assay is as follows:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
   i) the PBMCs are isolated using standard density centrifugation procedures and are suspended at 5×10⁶ cells/ml in RPMI cell culture medium;
   ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
   iii) 100 microliters of the final target cell suspension above are transferred to each well of a 96-well microtiter plate;
   iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
   v) for the maximum release (MR) controls, 3 additional wells in the plate containing the labeled target cells, receive 50 microliters of a 2% (v/v) aqueous solution of non-ionic detergent (Nonidet, Sigma, St. Louis), instead of the antibody solution (point iv above);
   vi) for the spontaneous release (SR) controls, 3 additional wells in the plate containing the labeled target cells, receive 50 microliters of RPMI cell culture medium instead of the antibody solution (point iv above);
   vii) the 96-well microtiter plate is then centrifuged at 50×g for 1 minute and incubated for 1 hour at 4° C.;
   viii) 50 microliters of the PBMC suspension (point i above) are added to each well to yield an effector:target cell ratio of 25:1 and the plates are placed in an incubator under 5% CO₂ atmosphere at 37° C. for 4 hours;
   ix) the cell-free supernatant from each well is harvested and the experimentally released radioactivity (ER) is quantified using a gamma counter;
   x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point v above), and SR is the average radioactivity quantified (see point ix above) for the SR controls (see point vi above);
4) “increased/reduced ADCC” is defined as either an increase/reduction in the maximum percentage of specific lysis observed within the antibody concentration range tested above, and/or a reduction/increase in the concentration of antibody required to achieve one half of the maximum percentage of specific lysis observed within the antibody concentration range tested above. The increase/reduction in ADCC is relative to the ADCC, measured with the above assay, mediated by the same antibody, produced by the same type of host cells, using the same standard production, purification, formulation and storage methods, which are known to those skilled in the art, but that has not been engineered.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

As used herein, the terms “first,” “second,” “third,” etc. with respect to antigen binding moieties or domains, are used for convenience of distinguishing when there is more than one of each type of moiety or domain. Use of these terms is not intended to confer a specific order or orientation unless explicitly so stated.

The terms “multispecific” and “bispecific” mean that the antigen binding molecule is able to specifically bind to at least two distinct antigenic determinants. Typically, a bispecific antigen binding molecule comprises two antigen binding sites, each of which is specific for a different antigenic determinant. In certain embodiments, a bispecific antigen binding molecule is capable of simultaneously binding two antigenic determinants, particularly two antigenic determinants expressed on two distinct cells.

The term “valent” or “valency” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e., one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

An “activating T cell antigen” as used herein refers to an antigenic determinant expressed by a T lymphocyte, particularly a cytotoxic T lymphocyte, which is capable of inducing or enhancing T cell activation upon interaction with an antigen binding molecule. Specifically, interaction of an antigen binding molecule with an activating T cell antigen may induce T cell activation by triggering the signaling cascade of the T cell receptor complex. An exemplary activating T cell antigen is CD3. In a particular embodiment the activating T cell antigen is CD3, particularly the epsilon subunit of CD3 (see UniProt no. P07766 (version 130), NCBI RefSeq no. NP_000724.1, for the human sequence; or UniProt no. Q951I5 (version 49), NCBI GenBank no. BAB71849.1, for the cynomolgus [Macaca fascicularis] sequence).

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating therapeutic agents used in the present invention are capable of inducing T cell activation. Suitable assays to measure T cell activation are known in the art described herein.

A “target cell antigen” as used herein refers to an antigenic determinant presented on the surface of a target cell, for example a cell in a tumor such as a cancer cell or a cell of the tumor stroma. In a particular embodiment, the target cell antigen is CD20, particularly human CD20 (see UniProt no. P11836).

A “B-cell antigen” as used herein refers to an antigenic determinant presented on the surface of a B lymphocyte, particularly a malignant B lymphocyte (in that case the antigen also being referred to as “malignant B-cell antigen”).

A “T-cell antigen” as used herein refers to an antigenic determinant presented on the surface of a T lymphocyte, particularly a cytotoxic T lymphocyte.

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g., a Fab molecule and an Fc domain subunit) are linked by peptide bonds, either directly or via one or more peptide linkers.

An “effective amount” of an agent refers to the amount that is necessary to result in a physiological change in the cell or tissue to which it is administered.

A “therapeutically effective amount” of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

By “therapeutic agent” is meant an active ingredient, e.g., of a pharmaceutical composition, that is administered to a subject in an attempt to alter the natural course of a disease in the subject being treated, and can be performed either for prophylaxis or during the course of clinical pathology. An “immunotherapeutic agent” refers to a therapeutic agent that is administered to a subject in an attempt to restore or enhance the subject's immune response, e.g., to a tumor.

The term “pharmaceutical composition” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

The term “package insert” or “instructions for use” is used to refer to instructions customarily included in commercial packages of therapeutic products that contain information about the indications, usage, dosage, administration, combination therapy, contraindications and/or warnings concerning the use of such therapeutic products.

The term “combination treatment” noted herein encompasses combined administration (where two or more therapeutic agents are included in the same or separate formulations), and separate administration, in which case, administration of an antibody as reported herein can occur prior to, simultaneously, and/or following, administration of the additional therapeutic agent or agents, preferably an antibody or antibodies.

By a “crossover” Fab molecule (also termed “Crossfab”) is meant a Fab molecule wherein the variable domains or the constant domains of the Fab heavy and light chain are exchanged (i.e., replaced by each other), i.e., the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e., comprising a heavy chain composed of the heavy chain variable and constant domains (VH-CH1, in N- to C-terminal direction), and a light chain composed of the light chain variable and constant domains (VL-CL, in N- to C-terminal direction).

The term “polynucleotide” refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA). The term “nucleic acid molecule” refers to any one or more nucleic acid segments, e.g., DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98%, or 99% identical to a nucleotide sequence of the present invention can be determined conventionally using known computer programs, such as the ones discussed above for polypeptides (e.g., ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The term “about” as used herein refers to the usual error range for the respective value readily known to the skilled person in this technical field. Reference to “about” a value or parameter herein includes (and describes) embodiments that are directed to that value or parameter per se.

By “B cell proliferative disorder” is meant a disease wherein the number of B cells in a patient is increased as compared to the number of B cells in a healthy subject, and particularly wherein the increase in the number of B cells is the cause or hallmark of the disease. A “CD20-positive B cell proliferative disorder” is a B cell proliferative disorder wherein B-cells, particularly malignant B-cells (in addition to normal B-cells), express CD20.

Exemplary B cell proliferation disorders include Non-Hodgkin lymphoma (NHL), diffuse large B-cell lymphoma (DLBCL; e.g., relapsed or refractory DLBCL not otherwise specified (NOS), high grade B cell lymphoma (HGBCL; e.g., HGBCL NOS, double-hit HGBCL, and triple-hit HGBCL), primary mediastinal large B-cell lymphoma (PMBCL), and DLBCL arising from FL (transformed FL; trFL)); follicular lymphoma (FL), including Grades 1-3b FL; mantle-cell lymphoma (MCL); and marginal zone lymphoma (MZL), including splenic, nodal or extra-nodal MZL. In one embodiment the CD20-positive B cell proliferative disorder is a relapsed or refractory NHL (e.g., a relapsed or refractory DLBCL, a relapsed or refractory FL, or a relapsed or refractory MCL).

“Refractory disease” is defined as no complete remission to first-line therapy. In one embodiment refractory disease defined as no response to or relapse within 6 months of prior therapy. In one embodiment refractory disease is characterized by one or more of the following: Progressive disease (PD) as best response to first-line therapy, Stable disease (SD) as best response after at least 4 cycles of first line therapy (e.g., 4 cycles of rituximab, cyclophosphamide, doxorubicin hydrochloride (hydroxydaunorubicin), vincristine sulfate (Oncovin), and prednisone, also abbreviated as R-CHOP), or Partial response (PR) as best response after at least 6 cycles, and biopsy-proven residual disease or disease progression after the partial response. “Relapsed disease” is defined as complete remission to first-line therapy. In one embodiment disease relapse is proven by biopsy. In one embodiment, patients have relapsed after or failed to respond to at least two prior systemic treatment regimens (including at least one prior regimen containing anthracycline, and at least one containing an anti CD20-directed therapy).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the individual or subject is a human. In one instance, each subject in a population of subjects is human. In one instance, each subject in a reference population of subjects is human.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, methods of the invention are used to delay development of a disease or to slow the progression of a disease.

As used herein, “delaying progression” of a disorder or disease means to defer, hinder, slow, retard, stabilize, and/or postpone development of the disease or disorder (e.g., a CD20-positive B cell proliferative disorder, e.g., NHL, e.g., DLBCL). This delay can be of varying length of time, depending on the history of the disease and/or individual being treated. As is evident to one skilled in the art, a sufficient or significant delay can, in effect, encompass prevention, in that the individual does not develop the disease. For example, in a late stage cancer, development of central nervous system (CNS) metastasis, may be delayed.

By “reduce” or “inhibit” is meant the ability to cause an overall decrease, for example, of 20%, 30%, 40%, 50%, 60%, 70%, 75%, 80%, 85%, 90%, 95%, or greater. For clarity the term includes also reduction to zero (or below the detection limit of the analytical method), i.e., complete abolishment or elimination. In certain embodiments, reduce or inhibit can refer to the reduction or inhibition of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with an anti-CD20/anti-CD3 bispecific antibody using the step-up dosing regimen of the invention relative to unchanging, preset dosing with the target dose of the bispecific antibody. In other embodiments, reduce or inhibit can refer to effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP). In other embodiments reduce or inhibit can refer to the symptoms of the CD20-positive B cell proliferative disorder being treated (e.g., an NHL (e.g., a DLBCL), an FL (e.g., a relapsed and/or refractor FL or a transformed FL), an MCL, a high-grade B cell lymphoma, or a PMLBCL), the presence or size of metastases, or the size of the primary tumor.

As used herein, “administering” is meant a method of giving a dosage of the pharmaceutical composition of an anti-CD20/anti-CD3 bispecific antibody to a subject. The pharmaceutical compositions described herein can be administered intravenously (e.g., by intravenous infusion).

As used herein, “buffer” refers to a buffered solution that resists changes in pH by the action of its acid-base conjugate components (also referred to herein as “buffering agents”). In some embodiments, the buffer of this invention has a pH in the range of from about 5 to about 6. Exemplary buffering agents for use in the invention include, but are not limited to, histidine (e.g., histidine HCl), an acetate, a phosphate, a succinate, or a combination thereof. In some embodiments, the histidine is histidine hydrochloride (histidine HCl), histidine acetate, sodium phosphate monobasic, sodium phosphate dibasic, sodium phosphate tribasic, potassium phosphate monobasic, potassium phosphate dibasic, potassium phosphate tribasic, or a mixture thereof.

The pharmaceutical compositions according to the invention may also comprise one or more tonicity agents. The term “tonicity agents” denotes pharmaceutically acceptable excipients used to modulate the tonicity of the formulation. The formulation can be hypotonic, isotonic or hypertonic. Isotonicity in general relates to the osmotic pressure of a solution, usually relative to that of human blood serum (around 250-350 mOsmol/kg). The formulation according to the invention can be hypotonic, isotonic or hypertonic but will preferably be isotonic. An isotonic formulation is liquid or liquid reconstituted from a solid form, e.g. from a lyophilized form, and denotes a solution having the same tonicity as some other solution with which it is compared, such as physiologic salt solution and the blood serum. Suitable tonicity agents comprise but are not limited to salts like sodium chloride or potassium chloride, glycerine and any component from the group of amino acids or sugars, in particular glucose. Tonicity agents are generally used in an amount of about ≥200 mM.

Within the stabilizers and tonicity agents there is a group of compounds which can function in both ways, i.e., they can at the same time be a stabilizer and a tonicity agent. Examples thereof can be found in the group of sugars, amino acids, polyols, cyclodextrines, polyethyleneglycols and salts. An example for a sugar which can at the same time be a stabilizer and a tonicity agent is trehalose.

As used herein, a “surfactant” refers to a surface-active agent, preferably a nonionic surfactant. Examples of surfactants herein include polysorbate (for example, polysorbate 20, polysorbate 40, polysorbate 60, polysorbate 65, polysorbate 80, polysorbate 85); poloxamer (e.g., poloxamer 188); TRITON®; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.); polyethyl glycol, polypropyl glycol, and copolymers of ethylene and propylene glycol (e.g., PLURONIC® type block copolymers, e.g., PLURONIC® F-68); and the like. In one embodiment, the surfactant herein is polysorbate 20 (PS20). In yet another embodiment, the surfactant herein is poloxamer 188 (P188).

A “preservative” is a compound which can be optionally included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl, and benzyl alcohol; alkyl parabens such as methyl or propyl paraben; catechol; resorcinol; cyclohexanol; 3-pentanol, and m-cresol. In one embodiment, the preservative herein is benzyl alcohol. In some embodiments, the formulation does not include a preservative.

A “stable” pharmaceutical composition is a pharmaceutical formulation in which the anti-CD20/anti-CD3 bispecific antibody (e.g., anti-CD20/anti-CD3 TCB, e.g., glofitamab) therein essentially retains its physical stability and/or chemical stability and/or biological activity upon storage. Preferably, the formulation essentially retains its physical and chemical stability, as well as its biological activity upon storage (e.g., frozen storage). The storage period is generally selected based on the intended shelf-life of the formulation. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected amount of light exposure and/or temperature for a selected time period. Stability can be evaluated qualitatively and/or quantitatively in a variety of different ways, including evaluation of aggregate formation (for example, using size exclusion chromatography, by measuring turbidity, and/or by visual inspection); evaluation of ROS formation (for example, by using a light stress assay or an 2,2′-azobis(2-amidinopropane) dihydrochloride (AAPH) stress assay); oxidation of specific amino acid residues of the anti-CD20/anti-CD3 bispecific antibody (for example, a Met residue of the anti-CD20/anti-CD3 bispecific antibody (e.g., anti-CD20/anti-CD3 TCB, e.g., glofitamab)); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact anti-CD20/anti-CD3 bispecific antibody; peptide map (for example, tryptic or LYS-C) analysis; evaluating biological activity or target binding function of the anti-CD20/anti-CD3 bispecific antibody (e.g., binding of to a T cell and/or a B cell); and the like. Instability may involve any one or more of: aggregation, deamidation (e.g., Asn deamidation), oxidation (e.g., Met oxidation and/or Trp oxidation), isomerization (e.g., Asp isomerization), clipping/hydrolysis/fragmentation (e.g., hinge region fragmentation), succinimide formation, unpaired cysteine(s), N-terminal extension, C-terminal processing, glycosylation differences, and the like. The term “liquid” as used herein in connection with the formulation according to the invention denotes a formulation which is liquid at a temperature of at least about 2 to about 8° C. under atmospheric pressure.

Within this application, unless otherwise stated, the techniques utilized may be found in any of several well-known references such as: Molecular Cloning: A Laboratory Manual (Sambrook, et al., 1989, Cold Spring Harbor Laboratory Press), PCR Protocols: A Guide to Methods and Applications (Innis, et al. 1990. Academic Press, San Diego, CA), and Harlow and Lane (1988) Antibodies: A Laboratory Manual ch.14 (Cold Spring Harbor Laboratory, Cold Spring Harbor, NY).

As appropriate, procedures involving the use of commercially available kits and reagents are generally carried out in accordance with manufacturer defined protocols and/or parameters unless otherwise noted. Before the present methods and uses therefore are described, it is to be understood that this invention is not limited to the particular methodology, protocols, cell lines, animal species or genera, constructs, and reagents described as such can, of course, vary. It is also to be understood that the terminology used herein is for the purpose of describing particular embodiments only, and is not intended to limit the scope of the present invention which will be limited only by the appended claims.

### III. Pharmaceutical Compositions

The invention provides pharmaceutical compositions that include anti-CD20/anti-CD3 bispecific antibodies (e.g., anti-CD20/anti-CD3 TCBs, e.g., glofitamab) at low concentrations and uses thereof, for example, for treatment of B-cell proliferative disorders (e.g., non-Hodgkin lymphoma, NHL). Pharmaceutical compositions of the invention can be formulated to support low concentrations of the anti-CD20/anti-CD3 bispecific antibody (e.g., anti-CD20/anti-CD3 TCB, e.g., glofitamab) and are stable against protein loss by adsorption during storage and clinical administration. Adsorption can be a significant issue for low antibody concentrations that require further dilution and handling prior to clinical administration and could result in low potency values. Glofitamab is given at a dose of 2.5 mg and 10 mg (step fractionated dose) and 30 mg maintenance dose (target dose, flat dose). Glofitamab is intended for IV administration after dilution in 0.9% or 0.45% sodium chloride via IV bag infusion. The doses are enabled in the IV bag by two dose solution concentrations from 0.05 mg/ml to 0.6 mg/ml.

In one embodiment, a liquid pharmaceutical composition is provided comprising:
about 1 to 25 mg/ml of an anti-CD20/anti-CD3 bispecific antibody (e.g., anti-CD20/anti-CD3 TCB, e.g., glofitamab);
about 10 to 50 mM of a buffering agent;
about ≥200 mM of a tonicity agent;
about 0-15 mM methionine; and
about 0.2 mg/ml of a surfactant;
at a pH in the range of from about 5.0 to about 6.0.
   In one embodiment, a liquid pharmaceutical composition is provided comprising:
about 5 mg/ml of an anti-CD20/anti-CD3 bispecific antibody (e.g., anti-CD20/anti-CD3 TCB, e.g., glofitamab);
about 10 to 50 mM of a buffering agent;
about ≥200 mM of a tonicity agent;
about 0-15 mM methionine; and
about 0.2 mg/ml of a surfactant;
at a pH in the range of from about 5.0 to about 6.0.
   In one embodiment, a liquid pharmaceutical composition is provided comprising:
about 0.9 to 1.1 mg/ml of an anti-CD20/anti-CD3 bispecific antibody (e.g., anti-CD20/anti-CD3 TCB, e.g., glofitamab);
about 10 to 50 mM of a buffering agent;
about ≥200 mM of a tonicity agent;
about 0-15 mM methionine; and
about ≥0.2 mg/ml of a surfactant;
at a pH in the range of from about 5.0 to about 6.0.
   In one embodiment, a liquid pharmaceutical composition is provided comprising:
about 1 mg/ml of an anti-CD20/anti-CD3 bispecific antibody (e.g., anti-CD20/anti-CD3 TCB, e.g., glofitamab);
about 10 to 50 mM of a buffering agent;
about ≥200 mM of a tonicity agent;
about 0-15 mM methionine; and
about 0.2 mg/ml of a surfactant;
at a pH in the range of from about 5.0 to about 6.0.

In one embodiment, the anti-CD20/anti-CD3 bispecific antibody concentration is in the range of about 1 to 5 mg/ml. In one embodiment, the anti-CD20/anti-CD3 bispecific antibody concentration is about 0.5, about 0.6, about 0.7, about 0.8, about 0.9, about 1 mg/ml, about 1.1 mg/ml, about 1.5 mg/ml, about 2 mg/ml, about 3 mg/ml, about 4 mg/ml or about 5 mg/ml. In one embodiment, the anti-CD20/anti-CD3 bispecific antibody concentration is about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, or about 30 mg/ml.

In one embodiment, the anti-CD20/anti-CD3 bispecific antibody concentration is in the range of about 0.9-1.1 mg/ml. In one embodiment, the anti-CD20/anti-CD3 bispecific antibody concentration is about 1 mg/ml.

In one embodiment the liquid pharmaceutical composition comprises an anti-CD20/anti-CD3 bispecific antibody (e.g., anti-CD20/anti-CD3 TCB, e.g., glofitamab) comprising at least one antigen binding domain that specifically binds to CD20, comprising a heavy chain variable region comprising
(i) an HVR-H1 comprising the amino acid sequence of SEQ ID NO: 1;
(ii) an HVR-H2 comprising the amino acid sequence of SEQ ID NO: 2; and
(iii) an HVR-H3 comprising the amino acid sequence of SEQ ID NO: 3;
   and a light chain variable region comprising
(i) an HVR-L1 comprising the amino acid sequence of SEQ ID NO: 4;
(ii) an HVR-L2 comprising the amino acid sequence of SEQ ID NO: 5; and
(iii) an HVR-L3 comprising the amino acid sequence of SEQ ID NO: 6.

In one embodiment, the anti-CD20/anti-CD3 bispecific antibody (e.g., anti-CD20/anti-CD3 TCB, e.g., glofitamab) of the liquid pharmaceutical composition comprises at least one antigen binding domain that specifically binds to CD20, comprising a heavy chain variable region sequence that is at least 80%, 85%, 90%, 95%, 96%, 97%, 98%, or 99% identical to of SEQ ID NO: 7 and a light chain variable region sequence that is at least 80%, 85%, 90%, 95%, 96%, 97%, 98%, or 99% identical to the sequence of SEQ ID NO: 8. In a further embodiment, the anti-CD20/anti-CD3 bispecific antibody (e.g., anti-CD20/anti-CD3 TCB, e.g., glofitamab) comprises at least one antigen binding domain that specifically binds to CD20 comprising the heavy chain variable region sequence of SEQ ID NO: 7 and the light chain variable region sequence of SEQ ID NO: 8.

In one embodiment, the anti-CD20/anti-CD3 bispecific antibody (e.g., anti-CD20/anti-CD3 TCB, e.g., glofitamab) of the liquid pharmaceutical composition comprises at least one antigen binding domain that specifically binds to CD3 comprising:

a heavy chain variable region comprising:
(i) an HVR-H1 comprising the amino acid sequence of SEQ ID NO: 9;
(ii) an HVR-H2 comprising the amino acid sequence of SEQ ID NO: 10; and
(iii) an HVR-H3 comprising the amino acid sequence of SEQ ID NO: 11;
   and a light chain variable region comprising
(i) an HVR-L1 comprising the amino acid sequence of SEQ ID NO: 12;
(ii) an HVR-L2 comprising the amino acid sequence of SEQ ID NO: 13; and
(iii) an HVR-L3 comprising the amino acid sequence of SEQ ID NO: 14.

In one embodiment, anti-CD20/anti-CD3 bispecific antibody (e.g., anti-CD20/anti-CD3 TCB, e.g., glofitamab) of the liquid pharmaceutical composition comprises at least one antigen binding domain that specifically binds to CD3, comprising a heavy chain variable region sequence that is at least 80%, 85%, 90%, 95%, 96%, 97%, 98%, or 99% identical to of SEQ ID NO: 15 and a light chain variable region sequence that is at least 80%, 85%, 90%, 95%, 96%, 97%, 98%, or 99% identical to the sequence of SEQ ID NO: 16. In a further embodiment, the anti-CD20/anti-CD3 bispecific antibody (e.g., anti-CD20/anti-CD3 TCB, e.g., glofitamab) comprises at least one antigen binding domain that specifically binds to CD3 comprising the heavy chain variable region sequence of SEQ ID NO: 15 and the light chain variable region sequence of SEQ ID NO: 16.

In one embodiment, the anti-CD20/anti-CD3 bispecific antibody (e.g., anti-CD20/anti-CD3 TCB, e.g., glofitamab) of the liquid pharmaceutical composition comprises
a) at least one antigen binding domain that specifically binds to CD20
   comprising a heavy chain variable region comprising:
      (i) an HVR-H1 comprising the amino acid sequence of SEQ ID NO: 1;
      (ii) an HVR-H2 comprising the amino acid sequence of SEQ ID NO: 2; and
      (iii) an HVR-H3 comprising the amino acid sequence of SEQ ID NO: 3;
and a light chain variable region comprising:
   (i) an HVR-L1 comprising the amino acid sequence of SEQ ID NO: 4;
   (ii) an HVR-L2 comprising the amino acid sequence of SEQ ID NO: 5; and
   (iii) an HVR-L3 comprising the amino acid sequence of SEQ ID NO: 6; and
b) at least one antigen binding domain that specifically binds to CD3
   comprising a heavy chain variable region comprising:
      (i) an HVR-H1 comprising the amino acid sequence of SEQ ID NO: 9;
      (ii) an HVR-H2 comprising the amino acid sequence of SEQ ID NO: 10; and
      (iii) an HVR-H3 comprising the amino acid sequence of SEQ ID NO: 11; and
a light chain variable region comprising:
   (i) an HVR-L1 comprising the amino acid sequence of SEQ ID NO: 12;
   (ii) an HVR-L2 comprising the amino acid sequence of SEQ ID NO: 13; and
   (iii) an HVR-L3 comprising the amino acid sequence of SEQ ID NO: 14.

In one embodiment, the anti-CD20/anti-CD3 bispecific antibody (e.g., anti-CD20/anti-CD3 TCB, e.g., glofitamab) of the liquid pharmaceutical composition comprises
(i) at least one antigen binding domain that specifically binds to CD20 comprising the heavy chain variable region sequence of SEQ ID NO: 7 and the light chain variable region sequence of SEQ ID NO: 8, and
(ii) at least one antigen binding domain that specifically binds to CD3 comprising the heavy chain variable region sequence of SEQ ID NO: 15 and the light chain variable region sequence of SEQ ID NO: 16.

In one embodiment, the antigen binding domain that specifically binds to CD3 of the anti-CD20/anti-CD3 bispecific antibody (e.g., anti-CD20/anti-CD3 TCB, e.g., glofitamab) is an antibody fragment, particularly a Fab molecule or a scFv molecule, more particularly a Fab molecule. In a particular embodiment, the antigen binding domain that specifically binds to CD3 of the anti-CD20/anti-CD3 bispecific antibody (e.g., anti-CD20/anti-CD3 TCB, e.g., glofitamab) is a crossover Fab molecule wherein the variable domains or the constant domains of the Fab heavy and light chain are exchanged (i.e., replaced by each other).

In one embodiment, the antigen binding domain that specifically binds to CD20 of the anti-CD20/anti-CD3 bispecific antibody (e.g., anti-CD20/anti-CD3 TCB, e.g., glofitamab) is an antibody fragment, particularly a Fab molecule or a scFv molecule, more particularly a Fab molecule. In a particular embodiment, the antigen binding domain that specifically binds to CD20 of the anti-CD20/anti-CD3 bispecific antibody (e.g., anti-CD20/anti-CD3 TCB, e.g., glofitamab) is a conventional Fab molecule.

In one embodiment, the anti-CD20/anti-CD3 bispecific antibody (e.g., anti-CD20/anti-CD3 TCB, e.g., glofitamab) of the liquid pharmaceutical composition comprises at least one antigen binding domain that specifically binds to CD20, and one antigen binding domain that specifically binds to CD3. In one embodiment, the anti-CD20/anti-CD3 bispecific antibody of the liquid pharmaceutical composition comprises a first antigen binding domain that specifically binds to CD3, and a second and a third antigen binding domain that specifically bind to CD20. In one embodiment, the first antigen binding domain is a crossover Fab molecule, and the second and the third antigen binding domain are each a conventional Fab molecule. In one embodiment, the anti-CD20/anti-CD3 bispecific antibody (e.g., anti-CD20/anti-CD3 TCB, e.g., glofitamab) further comprises an Fc domain. The anti-CD20/anti-CD3 bispecific antibody (e.g., anti-CD20/anti-CD3 TCB, e.g., glofitamab) of the liquid pharmaceutical composition may comprise modifications in the Fc region and/or the antigen binding domains as described herein. In one embodiment, the anti-CD20/anti-CD3 bispecific antibody (e.g., anti-CD20/anti-CD3 TCB, e.g., glofitamab) of the liquid pharmaceutical composition comprises an IgG1 Fc domain comprising one or more amino acid substitutions that reduce binding to an Fc receptor and/or effector function. In one embodiment the anti-CD20/anti-CD3 bispecific antibody (e.g., anti-CD20/anti-CD3 TCB, e.g., glofitamab) of the liquid pharmaceutical composition comprises an IgG1 Fc domain comprising the amino acid substitutions L234A, L235A and P329G (EU numbering).

In one embodiment the anti-CD20/anti-CD3 bispecific antibody (e.g., anti-CD20/anti-CD3 TCB, e.g., glofitamab) of the liquid pharmaceutical composition comprises
(i) an antigen binding domain that specifically binds to CD3 which is fused at the C-terminus of the Fab heavy chain to the N-terminus of the first subunit of the Fc domain,
(ii) a first antigen binding domain that specifically binds to CD20 which is fused at the C-terminus of the Fab heavy chain to the N-terminus of the Fab heavy chain of the antigen binding domain that specifically binds to CD3; and
(iii) a second antigen binding domain that specifically binds to CD20 which is fused at the C-terminus of the Fab heavy chain to the N-terminus of the second subunit of the Fc domain.

In a particular embodiment, the anti-CD20/anti-CD3 bispecific antibody (e.g., anti-CD20/anti-CD3 TCB, e.g., glofitamab) of the liquid pharmaceutical composition comprises
a) a first Fab molecule which specifically binds to CD3, particularly CD3 epsilon; and wherein the variable domains VL and VH of the Fab light chain and the Fab heavy chain are replaced by each other;
b) a second and a third Fab molecule which specifically bind to CD20, wherein in the constant domain CL of the second and third Fab molecule the amino acid at position 124 is substituted by lysine (K) (numbering according to Kabat) and the amino acid at position 123 is substituted by lysine (K) or arginine (R), particularly by arginine (R) (numbering according to Kabat), and wherein in the constant domain CH1 o of the second Fab and third Fab molecule the amino acid at position 147 is substituted by glutamic acid (E) (EU numbering) and the amino acid at position 213 is substituted by glutamic acid (E) (EU numbering); and
c) a Fc domain composed of a first and a second subunit capable of stable association.

In one embodiment, the anti-CD20/anti-CD3 bispecific antibody (e.g., anti-CD20/anti-CD3 TCB, e.g., glofitamab) of the liquid pharmaceutical composition comprises two antigen binding domains that specifically bind to CD20 and one antigen binding domain that specifically binds to CD3.

In one embodiment, the anti-CD20/anti-CD3 bispecific antibody (e.g., anti-CD20/anti-CD3 TCB, e.g., glofitamab) of the liquid pharmaceutical composition is bivalent for CD20 and monovalent for CD3.

In one embodiment the first Fab molecule under a) is fused at the C-terminus of the Fab heavy chain to the N-terminus of one of the subunits of the Fc domain under c), the second Fab molecule under b) is fused at the C-terminus of the Fab heavy chain to the N-terminus of the heavy chain of the first Fab molecule under a), and the third Fab molecule under b) is fused at the C-terminus of the Fab heavy chain to the N-terminus of the other subunit of the Fc domain under c). In one embodiment, the first Fab molecule under a) comprises a heavy chain variable region that is at least 95%, 96%, 97%, 98%, or 99% identical to the sequence of SEQ ID NO: 15, and a light chain variable region that is at least 95%, 96%, 97%, 98%, or 99% identical to the sequence of SEQ ID NO: 16.

In still a further embodiment, the first Fab molecule under a) comprises the heavy chain variable region sequence of SEQ ID NO: 15, and the light chain variable region sequence of SEQ ID NO: 16.

In one embodiment, the second Fab molecule and the third Fab molecule under b) each comprise a heavy chain variable region that is at least 95%, 96%, 97%, 98%, or 99% identical to the sequence of SEQ ID NO: 7, and a light chain variable region that is at least 95%, 96%, 97%, 98%, or 99% identical to the sequence of SEQ ID NO: 8.

In one embodiment, the second Fab molecule and the third Fab molecule under b) each comprise the heavy chain variable region sequence of SEQ ID NO: 7, and the light chain variable region sequence of SEQ ID NO: 8.

In a particular embodiment, the anti-CD20/anti-CD3 bispecific antibody (e.g., anti-CD20/anti-CD3 TCB, e.g., glofitamab) of the liquid pharmaceutical composition comprises a polypeptide that is at least 95%, 96%, 97%, 98%, or 99% identical to the sequence of SEQ ID NO: 17, a polypeptide that is at least 95%, 96%, 97%, 98%, or 99% identical to the sequence of SEQ ID NO: 18, a polypeptide that is at least 95%, 96%, 97%, 98%, or 99% identical to the sequence of SEQ ID NO: 19, and a polypeptide that is at least 95%, 96%, 97%, 98%, or 99% identical to the sequence of SEQ ID NO: 20. In a further particular embodiment, the bispecific antibody comprises a polypeptide sequence of SEQ ID NO: 17, a polypeptide sequence of SEQ ID NO: 18, a polypeptide sequence of SEQ ID NO: 19 and a polypeptide sequence of SEQ ID NO: 20. In a further particular embodiment, the bispecific antibody comprises one polypeptide chain comprising the amino acid sequence of SEQ ID NO: 17, one polypeptide chain comprising the amino acid sequence of SEQ ID NO: 18, one polypeptide chain comprising the amino acid sequence of SEQ ID NO: 19, and two polypeptide chains each comprising the amino acid sequence of SEQ ID NO: 20.

Particular anti-CD20/anti-CD3 bispecific antibodies are described in PCT publication no. WO 2016/020309 and European patent application nos. EP15188093 and EP16169160 (each incorporated herein by reference in its entirety).

In one embodiment, the anti-CD20/anti-CD3 bispecific antibody (e.g., anti-CD20/anti-CD3 TCB, e.g., glofitamab) of the liquid pharmaceutical composition specifically binds to CD3ε.

In one embodiment, the anti-CD20/anti-CD3 bispecific antibody of the liquid pharmaceutical composition can compete for binding with antibody H2C (PCT publication no. WO2008/119567), antibody V9 (Rodrigues et al., Int J Cancer Suppl. 7, 45-50 (1992) and U.S. Pat. No. 6,054,297), antibody FN18 (Nooij et al., Eur J Immunol. 19, 981-984 (1986)), antibody SP34 (Pessano et al., EMBO J. 4, 337-340 (1985)), antibody OKT3 (Kung et al., Science 206, 347-349 (1979)), antibody WT31 (Spits et al., J Immunol. 135, 1922 (1985)), antibody UCHT1 (Burns et al., J Immunol. 129, 1451-1457 (1982)), antibody 7D6 (Coulie et al., Eur J Immunol. 21, 1703-1709 (1991)) or antibody Leu-4. In some embodiments, the anti-CD20/anti-CD3 bispecific antibody of the liquid pharmaceutical composition may also comprise an antigen binding moiety that specifically binds to CD3 as described in WO 2005/040220, WO 2005/118635, WO 2007/042261, WO 2008/119567, WO 2008/119565, WO 2012/162067, WO 2013/158856, WO 2013/188693, WO 2013/186613, WO 2014/110601, WO 2014/145806, WO 2014/191113, WO 2014/047231, WO 2015/095392, WO 2015/181098, WO 2015/001085, WO 2015/104346, WO 2015/172800, WO 2016/020444, or WO 2016/014974.

In some embodiments, the anti-CD20/anti-CD3 bispecific antibody of the liquid pharmaceutical composition may comprise an antibody or an antigen binding moiety from rituximab, obinutuzumab, ocrelizumab, ofatumumab, ocaratuzumab, veltuzumab, and ublituximab.

In one embodiment, the anti-CD20/anti-CD3 bispecific antibody is glofitamab.

In some embodiments, the anti-CD20/anti-CD3 bispecific antibody may comprise a generic, biosimilar or non-comparable biologic version of an antibody, named herein.

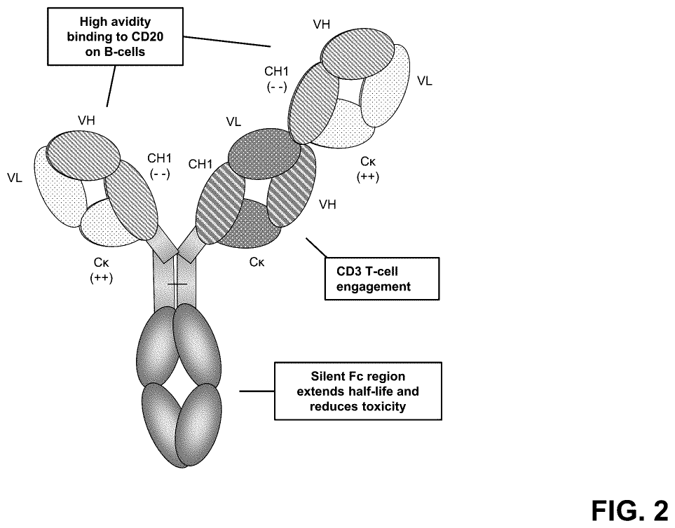

In one embodiment the anti-CD20/anti-CD3 bispecific antibody of the liquid pharmaceutical composition provided herein is glofitamab. Glofitamab (WHO Drug Information (International Nonproprietary Names for Pharmaceutical Substances), Recommended INN: List 83, 2020, vol. 34, no. 1, p. 39, also known as CD20-TCB, RO7082859, or RG6026; CAS #: 2229047-91-8) is a novel T-cell-engaging bispecific (TCB) full-length antibody with a 2:1 molecular configuration for bivalent binding to CD20 on B cells and monovalent binding to CD3, particularly the CD3 epsilon chain (CD3e), on T cells. Its CD3-binding region is fused to one of the CD20-binding regions in a head-to-tail fashion via a flexible linker. This structure endows glofitamab with superior in vitro potency versus other CD20-CD3 bispecific antibodies with a 1:1 configuration and leads to profound antitumor efficacy in preclinical DLBCL models. CD20 bivalency preserves this potency in the presence of competing anti-CD20 antibodies, providing the opportunity for pre- or co-treatment with these agents. Glofitamab comprises an engineered, heterodimeric Fc region with completely abolished binding to FcgRs and C1q. By simultaneously binding to human CD20-expressing tumor cells and to the CD3e of the T-cell receptor (TCR) complex on T-cells, it induces tumor cell lysis, in addition to T-cell activation, proliferation and cytokine release. Lysis of B-cells mediated by glofitamab is CD20-specific and does not occur in the absence of CD20 expression or in the absence of simultaneous binding (cross-linking) of T-cells to CD20-expressing cells. In addition to killing, T-cells undergo activation due to CD3 cross-linking, as detected by an increase in T-cell activation markers (CD25 and CD69), cytokine release (IFNγ, TNFα, IL-2, IL-6, IL-10), cytotoxic granule release (Granzyme B) and T-cell proliferation. A schematic of the molecule structure of glofitamab is depicted in FIG. 2. The sequences of glofitamab are summarized in Table 2.

TABLE 2
Sequence IDs for glofitamab
Sequence IDs for glofitamab

| SEQ ID NO: | Description | SEQ ID NO: | Description |
|---|---|---|---|
| CD3 Heavy Chain | | CD3 Light Chain | |
| 9 | HVR-H1 (Kabat) | 12 | HVR-L1 (Kabat) |
| 10 | HVR-H2 (Kabat) | 13 | HVR-L2 (Kabat) |
| 11 | HVR-H3 (Kabat) | 14 | HVR-L3 (Kabat) |
| 15 | VH | 16 | VL |
| CD20 Heavy Chain | | CD20 Light Chain | |
| 1 | HVR-H1 (Kabat) | 4 | HVR-L1 (Kabat) |
| 2 | HVR-H2 (Kabat) | 5 | HVR-L2 (Kabat) |
| 3 | HVR-H3 (Kabat) | 6 | HVR-L3 (Kabat) |
| 7 | VH | 8 | VH |

Full-length antibody

| | | | |
|---|---|---|---|
| 17 | HC-knob | 18 | HC-hole |
| 19 | LC-CD3 | 20 | LC-CD20 |

In some embodiments, the buffering agent is histidine, an acetate, a phosphate, a succinate, a citrate, or a combination thereof. In some embodiments, the histidine is a histidine acetate. Alternative buffering agents include sodium phosphate monobasic, sodium phosphate dibasic, sodium phosphate tribasic, potassium phosphate monobasic, potassium phosphate dibasic, potassium phosphate tribasic, or a mixture thereof. In a particular embodiment the liquid pharmaceutical composition comprises a histidine buffer, i.e., a buffer having histidine, generally L-histidine, as buffering agent. In a particular embodiment the buffering agent comprises L-histidine HCl, i.e., a buffer comprising L-histidine or mixtures of L-histidine and L-histidine HCl and pH adjustment achieved with hydrochloric acid. L-histidine HCl buffer can be prepared by dissolving suitable amounts of L-histidine and L-histidine hydrochloride in water, or by dissolving a suitable amount of L-histidine in water and adjusting the pH to the desired value by addition of hydrochloric acid.

In certain instances, the buffering agent (e.g., histidine, e.g., L-histidine HCl) is at a concentration from 10 mM to 50 mM. For example, the buffering agent can be from 10 mM to 15 mM, or from 15 mM to 20 mM, e.g., from 6 mM to 18 mM, from 7 mM to 16 mM, from 8 mM to 15 mM, or from 9 mM to 12 mM, e.g., about 10 mM, about 11 mM about 12 mM, about 13 mM, about 14 mM, about 15 mM, about 16 mM, about 17 mM, about 18 mM, about 19 mM, or about 20 mM. In particular instances, the concentration of the buffering agent (e.g., histidine, e.g., L-histidine HCl) is from about 15 to 25 mM. In one embodiment, the buffering agent (e.g., histidine, e.g., L-histidine HCl) is at a concentration of about 20 mM.

Regardless of the buffer used, the pH can be adjusted to a value in the range from about 5.0 to about 6.0, particularly to a pH of about 5.2 to about 5.8, with an acid or a base known in the art, e.g. hydrochloric acid, acetic acid, phosphoric acid, sulfuric acid and citric acid, sodium hydroxide and potassium hydroxide.

It was found by the inventors of the present invention that an anti-CD20/anti-CD3 bispecific antibody (e.g., anti-CD20/anti-CD3 TCB, e.g., glofitamab) comprising
a) at least one antigen binding domain that specifically binds to CD20
comprising a heavy chain variable region comprising:
   (i) an HVR-H1 comprising the amino acid sequence of SEQ ID NO: 1;
   (ii) an HVR-H2 comprising the amino acid sequence of SEQ ID NO: 2; and
   (iii) an HVR-H3 comprising the amino acid sequence of SEQ ID NO: 3;
and a light chain variable region comprising:
   (i) an HVR-L1 comprising the amino acid sequence of SEQ ID NO: 4;
   (ii) an HVR-L2 comprising the amino acid sequence of SEQ ID NO: 5; and
   (iii) an HVR-L3 comprising the amino acid sequence of SEQ ID NO: 6; and
b) at least one antigen binding domain that specifically binds to CD3
comprising a heavy chain variable region comprising:
   (i) an HVR-H1 comprising the amino acid sequence of SEQ ID NO: 9;
   (ii) an HVR-H2 comprising the amino acid sequence of SEQ ID NO: 10; and
   (iii) an HVR-H3 comprising the amino acid sequence of SEQ ID NO: 11; and
a light chain variable region comprising:
   (i) an HVR-L1 comprising the amino acid sequence of SEQ ID NO: 12;
   (ii) an HVR-L2 comprising the amino acid sequence of SEQ ID NO: 13; and
   (iii) an HVR-L3 comprising the amino acid sequence of SEQ ID NO: 14,
      is particularly stable in compositions at a pH of about 5.2 to about 5.8. In one embodiment the buffering agent provides a pH of about 5.2 to about 5.8, particularly a pH of about 5.5.

In some embodiments, the pharmaceutical composition includes a tonicity agent, such as a sugar, an amino acid, or a salt. In embodiments in which the tonicity agent is a sugar, the sugar can be, e.g., sucrose, glucose, glycerol or trehalose. In particular embodiments, the sugar is sucrose, optionally D-sucrose. In some embodiments the tonicity agent is either sucrose or sodium chloride. The tonicity agent (e.g., sugar, e.g., sucrose) can be at a concentration from at least about ≥200 mM. For example, the tonicity agent (e.g., sugar, e.g., sucrose) can be at a concentration, such as from 200 mM to 220 mM, from 220 mM to 240 mM, from 240 mM to 260 mM, from 260 mM to 280 mM, from 280 mM to 300 mM, from 300 mM to 320 mM, from 320 mM to 340 mM, from 340 mM to 360 mM, from 360 mM to 380 mM, from 380 mM to 400 mM, from 400 mM to 420 mM, from 420 mM to 440 mM, from 440 mM to 460 mM, from 460 mM to 480 mM, or from 480 mM to 500 mM, e.g., from 200 mM to 300 mM, e.g., about 200 mM, about 210 mM, about 220 mM, about 230 mM, about 240 mM, about 250 mM, about 260 mM, about 270 mM, about 280 mM, about 290 mM, about 300 mM, about 350 mM, about 400 mM, about 450 mM, or about 500 mM. In some embodiments, the concentration of the tonicity agent is about 200 mM to 280 mM. In some embodiments, the concentration of the tonicity agent is about 240 mM. In one particular embodiment, the tonicity agent is sucrose and present at a concentration of at least about 200 mM, i.e., at a concentration of about ≥200 mM. In other particular embodiments, the tonicity agent is sucrose (e.g., D-sucrose) and present at a concentration of about 200 mM-280 mM. In one particular embodiment, the tonicity agent is sucrose (e.g., D-sucrose) and present at a concentration of about 240 mM.

In some embodiments the liquid pharmaceutical composition comprises methionine as a stabilizer.

Any suitable concentration of the stabilizer methionine may be used. For example, in some embodiments of any of the preceding pharmaceutical compositions, the concentration of the stabilizer (e.g., methionine) is about 0.01 mM to about 15 mM, e.g., about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, or about 15 mM.

In particular embodiments, the concentration of methionine is from about 5 mM to 15 mM. In particular embodiments, the concentration of methionine is about 10 mM.

Any of the pharmaceutical compositions described herein can include a surfactant. Any suitable surfactant can be used. In some embodiments, the surfactant is a nonionic surfactant (e.g., a polysorbate (a polyoxyethylene (n) sorbitan monolaurate), a poloxamer, a polyoxyethelene alkyl ether, an alkyl phenyl polyoxyethylene ether, or a combination thereof). In some embodiments, the nonionic surfactant is a polysorbate (e.g., polysorbate 20 (polyoxyethylene (20) sorbitan monolaurate (PS20), TWEEN 20®; e.g., super refined PS20 (a PS20 that has been subjected to proprietary flash chromatographic process for greater purity and is available from Avantor Performance Materials, LLC (Center Valley, PA, US))) or polysorbate 80 (polyoxyethylene (20) sorbitan monooleate (PS80), e.g., TWEEN 80®; e.g., super refined PS80 (Avantor))). In particular embodiments, the polysorbate is polysorbate 20. In other embodiments, the nonionic surfactant is a poloxamer (e.g., poloxamer 188, poly(ethylene glycol)-block-poly(propylene glycol)-block-poly(ethylene glycol)).

The pharmaceutical surfactant can be at a concentration from at least about 0.2 mg/ml, i.e., at a concentration from at least about 0.02% (w/v).

In some embodiments of any of the pharmaceutical compositions described herein, the concentration of the surfactant (e.g., PS20 or P188) is about 0.01% (w/v) to about 2% (w/v), e.g., about 0.01%, about 0.02%, about 0.03%, about 0.04%, about 0.05%, about 0.06%, about 0.07%, about 0.08%, about 0.09%, about 0.1%, about 0.15%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% (w/v).

In some embodiments, the concentration of the surfactant (e.g., PS20 or P188) is about 0.1-1 mg/ml, i.e., 0.01% (w/v) to about 0.1% (w/v). In some embodiments, the concentration of the surfactant (e.g., PS20 or P188) is about 0.2-1 mg/ml, i.e., 0.02% (w/v) to about 0.1% (w/v). In some embodiments, the concentration of the surfactant (e.g., PS20 or P188) is about 0.2-0.8 mg/ml, i.e., 0.02% (w/v) to about 0.08% (w/v). In some embodiments, the concentration of the surfactant (e.g., PS20 or P188) is about 0.5 mg/ml, i.e., 0.05% (w/v).

In certain embodiments, the surfactant is P188, and the concentration of the P188 is about 0.05% (w/v), 0.07% (w/v) or 0.1% (w/v).

In particular embodiments, the surfactant is PS20, and the concentration of PS20 is at least about 0.2 mg/ml, i.e at a concentration from at least about 0.02% (w/v) PS20.

In particular embodiments, the surfactant is PS20, and the concentration of PS20 is about 0.2-0.8 mg/ml, i.e., about 0.02% (w/v) to about 0.08% (w/v). In particular embodiments, the surfactant is PS20, and the concentration of PS20 is about 0.5 mg/ml, i.e., 0.05% (w/v). In particular embodiments, the surfactant is PS20, and the concentration of PS20 is at least about 0.02% (w/v) PS20. In particular embodiments, the surfactant is PS20, and the concentration of PS20 is about 0.02% (w/v) to about 0.08% (w/v). In particular embodiments, the surfactant is PS20, and the concentration of PS20 is about 0.05% (w/v).

In one embodiment, the liquid pharmaceutical composition according to the invention comprises:
about 1 to 5 mg/ml of an anti-CD20/anti-CD3 bispecific antibody (e.g., anti-CD20/anti-CD3 TCB, e.g., glofitamab) comprising:
   a) at least one antigen binding domain that specifically binds to CD20
   comprising a heavy chain variable region comprising:
      (i) an HVR-H1 comprising the amino acid sequence of SEQ ID NO: 1;
      (ii) an HVR-H2 comprising the amino acid sequence of SEQ ID NO: 2; and
      (iii) an HVR-H3 comprising the amino acid sequence of SEQ ID NO: 3;
      and a light chain variable region comprising:
         (i) an HVR-L1 comprising the amino acid sequence of SEQ ID NO: 4;
         (ii) an HVR-L2 comprising the amino acid sequence of SEQ ID NO: 5; and
         (iii) an HVR-L3 comprising the amino acid sequence of SEQ ID NO: 6; and
   b) at least one antigen binding domain that specifically binds to CD3
      comprising a heavy chain variable region comprising:
         (i) an HVR-H1 comprising the amino acid sequence of SEQ ID NO: 9;
         (ii) an HVR-H2 comprising the amino acid sequence of SEQ ID NO: 10; and
         (iii) an HVR-H3 comprising the amino acid sequence of SEQ ID NO: 11; and
      a light chain variable region comprising:
         (i) an HVR-L1 comprising the amino acid sequence of SEQ ID NO: 12;
         (ii) an HVR-L2 comprising the amino acid sequence of SEQ ID NO: 13; and
         (iii) an HVR-L3 comprising the amino acid sequence of SEQ ID NO: 14;
about 15-25 mM of a histidine buffer;
about 200-280 mM sucrose;
about 0-15 mM methionine; and
about 0.2-0.8 mg/ml of PS20
at a pH of about 5 to about 6.

In one embodiment, the liquid pharmaceutical composition according to the invention comprises:
about 1 to 5 mg/ml of an anti-CD20/anti-CD3 bispecific antibody (e.g., anti-CD20/anti-CD3 TCB, e.g., glofitamab) comprising:
   a) at least one antigen binding domain that specifically binds to CD20
      comprising a heavy chain variable region comprising:
         (i) an HVR-H1 comprising the amino acid sequence of SEQ ID NO: 1;
         (ii) an HVR-H2 comprising the amino acid sequence of SEQ ID NO: 2; and
         (iii) an HVR-H3 comprising the amino acid sequence of SEQ ID NO: 3;
      and a light chain variable region comprising:
         (i) an HVR-L1 comprising the amino acid sequence of SEQ ID NO: 4;
         (ii) an HVR-L2 comprising the amino acid sequence of SEQ ID NO: 5; and
         (iii) an HVR-L3 comprising the amino acid sequence of SEQ ID NO: 6; and
   b) at least one antigen binding domain that specifically binds to CD3
      comprising a heavy chain variable region comprising:
         (i) an HVR-H1 comprising the amino acid sequence of SEQ ID NO: 9;
         (ii) an HVR-H2 comprising the amino acid sequence of SEQ ID NO: 10; and
         (iii) an HVR-H3 comprising the amino acid sequence of SEQ ID NO: 11; and
      a light chain variable region comprising:
         (i) an HVR-L1 comprising the amino acid sequence of SEQ ID NO: 12;
         (ii) an HVR-L2 comprising the amino acid sequence of SEQ ID NO: 13; and
         (iii) an HVR-L3 comprising the amino acid sequence of SEQ ID NO: 14;
about 15-25 mM of a histidine buffer;
about 200-280 mM sucrose;
about 0-15 mM methionine; and
about 0.2-0.8 mg/ml of PS20
at a pH of about 5.2 to about 5.8.

In one embodiment, the liquid pharmaceutical composition according to the invention comprises:
about 0.9 to 1.1 mg/ml of an anti-CD20/anti-CD3 bispecific antibody (e.g., anti-CD20/anti-CD3 TCB, e.g., glofitamab) comprising:
   a) at least one antigen binding domain that specifically binds to CD20
      comprising a heavy chain variable region comprising:
         (i) an HVR-H1 comprising the amino acid sequence of SEQ ID NO: 1;
         (ii) an HVR-H2 comprising the amino acid sequence of SEQ ID NO: 2; and
         (iii) an HVR-H3 comprising the amino acid sequence of SEQ ID NO: 3;
      and a light chain variable region comprising:
         (i) an HVR-L1 comprising the amino acid sequence of SEQ ID NO: 4;
         (ii) an HVR-L2 comprising the amino acid sequence of SEQ ID NO: 5; and
         (iii) an HVR-L3 comprising the amino acid sequence of SEQ ID NO: 6; and
   b) at least one antigen binding domain that specifically binds to CD3
      comprising a heavy chain variable region comprising:
         (i) an HVR-H1 comprising the amino acid sequence of SEQ ID NO: 9;
         (ii) an HVR-H2 comprising the amino acid sequence of SEQ ID NO: 10; and
         (iii) an HVR-H3 comprising the amino acid sequence of SEQ ID NO: 11; and
      a light chain variable region comprising:
         (i) an HVR-L1 comprising the amino acid sequence of SEQ ID NO: 12;
         (ii) an HVR-L2 comprising the amino acid sequence of SEQ ID NO: 13; and
         (iii) an HVR-L3 comprising the amino acid sequence of SEQ ID NO: 14;
about 15-25 mM of a histidine buffer;
about 200-280 mM sucrose;
about 0-15 mM methionine; and
about 0.2-0.8 mg/ml of PS20
at a pH of about 5.2 to about 5.8.

In one embodiment the liquid pharmaceutical composition comprises:
about 1 mg/ml of an anti-CD20/anti-CD3 bispecific antibody (e.g., anti-CD20/anti-CD3 TCB, e.g., glofitamab) comprising:
   a) at least one antigen binding domain that specifically binds to CD20
      comprising a heavy chain variable region comprising:
         (i) an HVR-H1 comprising the amino acid sequence of SEQ ID NO: 1;
         (ii) an HVR-H2 comprising the amino acid sequence of SEQ ID NO: 2; and
         (iii) an HVR-H3 comprising the amino acid sequence of SEQ ID NO: 3;
      and a light chain variable region comprising:
         (i) an HVR-L1 comprising the amino acid sequence of SEQ ID NO: 4;
         (ii) an HVR-L2 comprising the amino acid sequence of SEQ ID NO: 5; and
         (iii) an HVR-L3 comprising the amino acid sequence of SEQ ID NO: 6; and
   b) at least one antigen binding domain that specifically binds to CD3
      comprising a heavy chain variable region comprising:
         (i) an HVR-H1 comprising the amino acid sequence of SEQ ID NO: 9;
         (ii) an HVR-H2 comprising the amino acid sequence of SEQ ID NO: 10; and
         (iii) an HVR-H3 comprising the amino acid sequence of SEQ ID NO: 11; and
      a light chain variable region comprising:
         (i) an HVR-L1 comprising the amino acid sequence of SEQ ID NO: 12;
         (ii) an HVR-L2 comprising the amino acid sequence of SEQ ID NO: 13; and
         (iii) an HVR-L3 comprising the amino acid sequence of SEQ ID NO: 14;
about 15-25 mM of a histidine buffer;
about 200-280 mM sucrose;
about 0-15 mM methionine; and
about 0.2-0.8 mg/ml of PS20
at a pH of about 5.2 to about 5.8.

In one embodiment, the liquid pharmaceutical composition comprises:
about 1 mg/ml of an anti-CD20/anti-CD3 bispecific antibody (e.g., anti-CD20/anti-CD3 TCB, e.g., glofitamab) comprising:
   a) at least one antigen binding domain that specifically binds to CD20
      comprising a heavy chain variable region comprising:
         (i) an HVR-H1 comprising the amino acid sequence of SEQ ID NO: 1;
         (ii) an HVR-H2 comprising the amino acid sequence of SEQ ID NO: 2; and
         (iii) an HVR-H3 comprising the amino acid sequence of SEQ ID NO: 3;
      and a light chain variable region comprising:
         (i) an HVR-L1 comprising the amino acid sequence of SEQ ID NO: 4;
         (ii) an HVR-L2 comprising the amino acid sequence of SEQ ID NO: 5; and
         (iii) an HVR-L3 comprising the amino acid sequence of SEQ ID NO: 6; and
   b) at least one antigen binding domain that specifically binds to CD3
      comprising a heavy chain variable region comprising:
         (i) an HVR-H1 comprising the amino acid sequence of SEQ ID NO: 9;
         (ii) an HVR-H2 comprising the amino acid sequence of SEQ ID NO: 10; and
         (iii) an HVR-H3 comprising the amino acid sequence of SEQ ID NO: 11; and
      a light chain variable region comprising:
         (i) an HVR-L1 comprising the amino acid sequence of SEQ ID NO: 12;
         (ii) an HVR-L2 comprising the amino acid sequence of SEQ ID NO: 13; and
         (iii) an HVR-L3 comprising the amino acid sequence of SEQ ID NO: 14;
about 20 mM of a histidine buffer;
about 240 mM sucrose;
about 10 mM methionine; and
about 0.5 mg/ml of PS20
at a pH of about 5.5.

In one embodiment, the liquid pharmaceutical composition according to the invention comprises:
about 1 to 5 mg/ml of an anti-CD20/anti-CD3 bispecific antibody (e.g., anti-CD20/anti-CD3 TCB, e.g., glofitamab) comprising:
   (i) at least one antigen binding domain that specifically binds to CD20 comprising the heavy chain variable region sequence of SEQ ID NO: 7 and the light chain variable region sequence of SEQ ID NO: 8, and
   (ii) at least one antigen binding domain that specifically binds to CD3 comprising the heavy chain variable region sequence of SEQ ID NO: 15 and the light chain variable region sequence of SEQ ID NO: 16;
about 15-25 mM of a histidine buffer;
about 200-280 mM sucrose;
about 0-15 mM methionine; and
about 0.2-0.8 mg/ml of PS20
at a pH of about 5.2 to about 5.8.

In one embodiment, the liquid pharmaceutical composition according to the invention comprises:
about 0.9 to about 1.1 mg/ml of an anti-CD20/anti-CD3 bispecific antibody comprising:
   (i) at least one antigen binding domain that specifically binds to CD20 comprising the heavy chain variable region sequence of SEQ ID NO: 7 and the light chain variable region sequence of SEQ ID NO: 8, and
   (ii) at least one antigen binding domain that specifically binds to CD3 comprising the heavy chain variable region sequence of SEQ ID NO: 15 and the light chain variable region sequence of SEQ ID NO: 16;
about 15-25 mM of a histidine buffer;
about 200-280 mM sucrose;
about 0-15 mM methionine; and
about 0.2-0.8 mg/ml of PS20
at a pH of about 5.2 to about 5.8.

In one embodiment, the liquid pharmaceutical composition according to the invention comprises:
about 1 mg/ml of an anti-CD20/anti-CD3 bispecific antibody (e.g., anti-CD20/anti-CD3 TCB, e.g., glofitamab) comprising:
   (i) at least one antigen binding domain that specifically binds to CD20 comprising the heavy chain variable region sequence of SEQ ID NO: 7 and the light chain variable region sequence of SEQ ID NO: 8, and
   (ii) at least one antigen binding domain that specifically binds to CD3 comprising the heavy chain variable region sequence of SEQ ID NO: 15 and the light chain variable region sequence of SEQ ID NO: 16;
about 15-25 mM of a histidine buffer;
about 200-280 mM sucrose;
about 0-15 mM methionine; and
about 0.2-0.8 mg/ml of PS20
at a pH of about 5.2 to about 5.8.

In one embodiment, the liquid pharmaceutical composition according to the invention comprises:
about 1 mg/ml of an anti-CD20/anti-CD3 bispecific antibody (e.g., anti-CD20/anti-CD3 TCB, e.g., glofitamab) comprising:
   (i) at least one antigen binding domain that specifically binds to CD20 comprising the heavy chain variable region sequence of SEQ ID NO: 7 and the light chain variable region sequence of SEQ ID NO: 8, and
   (ii) at least one antigen binding domain that specifically binds to CD3 comprising the heavy chain variable region sequence of SEQ ID NO: 15 and the light chain variable region sequence of SEQ ID NO: 16;
about 20 mM of a histidine buffer;
about 240 mM sucrose;
about 10 mM methionine; and
about 0.5 mg/ml of PS20
at a pH of about 5.5.

In one embodiment, the liquid pharmaceutical composition according to the invention comprises:
about 1 to 5 mg/ml of glofitamab,
about 15-25 mM of a histidine buffer;
about 200-280 mM sucrose;
about 0-15 mM methionine; and
about 0.2-0.8 mg/ml of PS20
at a pH of about 5.2 to about 5.8.

In one embodiment, the liquid pharmaceutical composition according to the invention comprises:
about 0.9 to about 1.1 mg/ml of glofitamab,
about 15-25 mM of a histidine buffer;
about 200-280 mM sucrose;
about 0-15 mM methionine; and
about 0.2-0.8 mg/ml of PS20
at a pH of about 5.2 to about 5.8.

In one embodiment, the liquid pharmaceutical composition according to the invention comprises:
about 1 mg/ml of glofitamab;
about 15-25 mM of a histidine buffer;
about 200-280 mM sucrose;
about 0-15 mM methionine; and
about 0.2-0.8 mg/ml of PS20
at a pH of about 5.2 to about 5.8.

In one embodiment, the liquid pharmaceutical composition according to the invention comprises:
about 1 mg/ml of glofitamab;
about 20 mM of a histidine buffer;
about 240 mM sucrose;
about 10 mM methionine; and
about 0.5 mg/ml of PS20
at a pH of about 5.5.

The formulations may also contain adjuvants such as preservatives, wetting agents, emulsifying agents, and dispersing agents. Prevention of presence of microorganisms may be ensured both by sterilization procedures, and by the inclusion of various antibacterial and antifungal agents, e.g., paraben, chlorobutanol, phenol, sorbic acid, and the like. Preservatives are generally used in an amount of about 0.001 to about 2% (w/v). Preservatives comprise but are not limited to ethanol, benzyl alcohol, phenol, m-cresol, p-chlor-m-cresol, methyl or propyl parabens, and benzalkonium chloride.

### IV. Therapeutic Agents for Use in the Pharmaceutical Compositions of the Invention

### A. Anti-CD20/Anti-CD3 Bispecific Antibodies

The present invention provides new pharmaceutical compositions of anti-CD20/anti-CD3 bispecific antibodies (e.g., anti-CD20/anti-CD3 T cell-engaging bispecific antibodies (TCBs), e.g., glofitamab). In one embodiment, the antibody is a monoclonal antibody. In one embodiment, the anti-CD20/anti-CD3 bispecific antibody is a polyclonal antibody. In one embodiment the anti-CD20/anti-CD3 bispecific antibody s a human antibody. In one embodiment, the anti-CD20/anti-CD3 bispecific antibody (e.g., anti-CD20/anti-CD3 TCB, e.g., glofitamab) is humanized antibody. In one embodiment the anti-CD20/anti-CD3 bispecific antibody is a chimeric antibody. In one embodiment the anti-CD20/anti-CD3 bispecific antibody is a full-length antibody. In one embodiment the anti-CD20/anti-CD3 bispecific antibody (e.g., anti-CD20/anti-CD3 TCB, e.g., glofitamab) is an IgG-class antibody, particularly an IgG1 subclass antibody. In one embodiment, the anti-CD20/anti-CD3 bispecific antibody (e.g., anti-CD20/anti-CD3 TCB, e.g., glofitamab) is a recombinant antibody.

In certain embodiments, the anti-CD20/anti-CD3 bispecific antibody comprises an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. In one embodiment, the antibody fragment is a Fab fragment or a scFv fragment.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

In certain embodiments, the anti-CD20/anti-CD3 bispecific antibody is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, the anti-CD20/anti-CD3 bispecific antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In certain embodiments, the anti-CD20/anti-CD3 bispecific antibody is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al. Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Binding domains comprised in the anti-CD20/anti-CD3 bispecific antibody (e.g., anti-CD20/anti-CD3 TCB, e.g., glofitamab) may be isolated by screening combinatorial libraries for binding moieties with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

Techniques for making bispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The anti-CD20/anti-CD3 bispecific antibody herein also includes a “Dual Acting FAb” or “DAF” comprising an antigen binding site that binds to two different antigens (see, US 2008/0069820, for example).

“Crossmab” antibodies are also included herein (see e.g., WO2009080251, WO2009080252, WO2009080253, WO2009080254).

Another technique for making bispecific antibody fragments is the “bispecific T cell engager” or BiTE® approach (see, e.g., WO2004/106381, WO2005/061547, WO2007/042261, and WO2008/119567). This approach utilizes two antibody variable domains arranged on a single polypeptide. For example, a single polypeptide chain includes two single chain Fv (scFv) fragments, each having a variable heavy chain (VH) and a variable light chain (VL) domain separated by a polypeptide linker of a length sufficient to allow intramolecular association between the two domains. This single polypeptide further includes a polypeptide spacer sequence between the two scFv fragments. Each scFv recognizes a different epitope, and these epitopes may be specific for different cell types, such that cells of two different cell types are brought into proximity or tethered when each scFv is engaged with its cognate epitope. One particular embodiment of this approach includes a scFv recognizing a cell-surface antigen expressed by an immune cell, e.g., a CD3 polypeptide on a T cell, linked to another scFv that recognizes a cell-surface antigen expressed by a target cell, such as a malignant or tumor cell.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species.

In certain embodiments, the anti-CD20/anti-CD3 bispecific antibody may be further modified to contain additional nonproteinaceous moieties that are known in the art and readily available. The moieties suitable for derivatization of the anti-CD20/anti-CD3 bispecific antibody (e.g., anti-CD20/anti-CD3 TCB, e.g., glofitamab) include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

The anti-CD20/anti-CD3 bispecific antibody may also be conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, the anti-CD20/anti-CD3 bispecific antibody comprises an antibody-drug conjugate (ADC) in which an antibody is conjugated to one or more drugs, including but not limited to a maytansinoid (see U.S. Pat. Nos. 5,208,020, 5,416,064, and European Patent EP 0425235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, the anti-CD20/anti-CD3 bispecific antibody is conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes.

In another embodiment, the anti-CD20/anti-CD3 bispecific antibody is conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example Tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of the anti-CD20/anti-CD3 bispecific antibody and a cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of a radionucleotide to an antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

In one embodiment, the anti-CD20/anti-CD3 bispecific antibody (e.g., anti-CD20/anti-CD3 TCB, e.g., glofitamab) is indicated for the treatment of a cell proliferative disorder (e.g., cancer). In one embodiment, the cell proliferative disorder is a cancer. In one embodiment, cancer is a B-cell proliferative disorder. In one embodiment, the cancer is a CD20-positive B-cell proliferative disorder. In one embodiment, the cancer is a non-Hodgkin's lymphoma (NHL). In one embodiment the NHL is a diffuse large B cell lymphoma (DLBCL), a high grade B cell lymphoma (HGBCL), a DLBCL arising from follicular lymphoma (FL) [transformed FL; trFL], a primary mediastinal large B-cell lymphoma (PMBCL), or marginal zone lymphoma (MZL). MZL can be categorized as splenic, nodal and extra-nodal MZL. In one embodiment the NHL is a mantle cell lymphoma (MCL). In one embodiment, the NHL is a Grades 1-3a Follicular Lymphoma (FL). In one embodiment, the CD20-positive B cell proliferative disorder is a relapsed or refractory B cell proliferative disorder. In one embodiment, the relapsed or refractory B cell proliferative disorder is relapsed or refractory NHL (e.g., a relapsed or refractory DLBCL, a relapsed or refractory FL, or a relapsed or refractory MCL).

In one embodiment, the anti-CD20/anti-CD3 bispecific antibody (e.g., anti-CD20/anti-CD3 TCB, e.g., glofitamab) specifically binds to CD3s.

In one embodiment, the anti-CD20/anti-CD3 bispecific antibody can compete for binding with antibody H2C (PCT Publication No. WO 2008/119567), antibody V9 (Rodrigues et al., Int J Cancer Suppl. 7, 45-50 (1992) and U.S. Pat. No. 6,054,297), antibody FN18 (Nooij et al., Eur J Immunol. 19, 981-984 (1986)), antibody SP34 (Pessano et al., EMBO J. 4, 337-340 (1985)), antibody OKT3 (Kung et al., Science 206, 347-349 (1979)), antibody WT31 (Spits et al., J Immunol. 135, 1922 (1985)), antibody UCHT1 (Burns et al., J Immunol. 129, 1451-1457 (1982)), antibody 7D6 (Coulie et al., Eur J Immunol. 21, 1703-1709 (1991)) or antibody Leu-4. In some embodiments, the anti-CD20/anti-CD3 bispecific antibody may also comprise an antigen binding moiety that specifically binds to CD3 as described in WO 2005/040220, WO 2005/118635, WO 2007/042261, WO 2008/119567, WO 2008/119565, WO 2012/162067, WO 2013/158856, WO 2013/188693, WO 2013/186613, WO 2014/110601, WO 2014/145806, WO 2014/191113, WO 2014/047231, WO 2015/095392, WO 2015/181098, WO 2015/001085, WO 2015/104346, WO 2015/172800, WO 2016/020444, or WO 2016/014974.

In some embodiments, the anti-CD20/anti-CD3 bispecific antibody may comprise an antibody or an antigen binding moiety from rituximab, obinutuzumab ocrelizumab, ofatumumab, ocaratuzumab, veltuzumab, and ublituximab.

In one embodiment, the anti-CD20/anti-CD3 bispecific antibody is glofitamab.

In some embodiments, the anti-CD20/anti-CD3 bispecific antibody may comprise a generic, biosimilar or non-comparable biologic version of an antibody, named herein.

In one embodiment, the anti-CD20/anti-CD3 bispecific antibody (e.g., anti-CD20/anti-CD3 TCB, e.g., glofitamab) comprises at least one antigen binding domain that specifically binds to CD20, comprising:
a heavy chain variable region comprising:
   (i) an HVR-H1 comprising the amino acid sequence of SEQ ID NO: 1;
   (ii) an HVR-H2 comprising the amino acid sequence of SEQ ID NO: 2; and
   (iii) an HVR-H3 comprising the amino acid sequence of SEQ ID NO: 3;
and a light chain variable region comprising:
   (i) an HVR-L1 comprising the amino acid sequence of SEQ ID NO: 4;
   (ii) an HVR-L2 comprising the amino acid sequence of SEQ ID NO: 5; and
   (iii) an HVR-L3 comprising the amino acid sequence of SEQ ID NO: 6.

In one embodiment, anti-CD20/anti-CD3 bispecific antibody (e.g., anti-CD20/anti-CD3 TCB, e.g., glofitamab) comprises at least one antigen binding domain that specifically binds to CD20, comprising a heavy chain variable region sequence that is at least 80%, 85%, 90%, 95%, 96%, 97%, 98%, or 99% identical to of SEQ ID NO: 7 and a light chain variable region sequence that is at least 80%, 85%, 90%, 95%, 96%, 97%, 98%, or 99% identical to the sequence of SEQ ID NO: 8. In a further embodiment, the anti-CD20/anti-CD3 bispecific antibody (e.g., anti-CD20/anti-CD3 TCB, e.g., glofitamab) comprises at least one antigen binding domain that specifically binds to CD20 comprising the heavy chain variable region sequence of SEQ ID NO: 7 and the light chain variable region sequence of SEQ ID NO: 8.

In one embodiment, the anti-CD20/anti-CD3 bispecific antibody (e.g., anti-CD20/anti-CD3 TCB, e.g., glofitamab) comprises at least one antigen binding domain that specifically binds to CD3 comprising
a heavy chain variable region comprising:
   (i) an HVR-H1 comprising the amino acid sequence of SEQ ID NO: 9;
   (ii) an HVR-H2 comprising the amino acid sequence of SEQ ID NO: 10; and
   (iii) an HVR-H3 comprising the amino acid sequence of SEQ ID NO: 11; and
and a light chain variable region comprising:
   (i) an HVR-L1 comprising the amino acid sequence of SEQ ID NO: 12;
   (ii) an HVR-L2 comprising the amino acid sequence of SEQ ID NO: 13; and
   (iii) an HVR-L3 comprising the amino acid sequence of SEQ ID NO: 14.

In one embodiment, anti-CD20/anti-CD3 bispecific antibody (e.g., anti-CD20/anti-CD3 TCB, e.g., glofitamab) comprises at least one antigen binding domain that specifically binds to CD3, comprising a heavy chain variable region sequence that is at least 80%, 85%, 90%, 95%, 96%, 97%, 98%, or 99% identical to of SEQ ID NO: 15 and a light chain variable region sequence that is at least 80%, 85%, 90%, 95%, 96%, 97%, 98%, or 99% identical to the sequence of SEQ ID NO: 16. In a further embodiment, the anti-CD20/anti-CD3 bispecific antibody (e.g., anti-CD20/anti-CD3 TCB, e.g., glofitamab) comprises at least one antigen binding domain that specifically binds to CD3 comprising the heavy chain variable region sequence of SEQ ID NO: 15 and the light chain variable region sequence of SEQ ID NO: 16.

In one embodiment, the anti-CD20/anti-CD3 bispecific antibody (e.g., anti-CD20/anti-CD3 TCB, e.g., glofitamab) comprises:
a) at least one antigen binding domain that specifically binds to CD20 comprising a heavy chain variable region comprising:
   (i) an HVR-H1 comprising the amino acid sequence of SEQ ID NO: 1;
   (ii) an HVR-H2 comprising the amino acid sequence of SEQ ID NO: 2; and
   (iii) an HVR-H3 comprising the amino acid sequence of SEQ ID NO: 3;
and a light chain variable region comprising:
   (i) an HVR-L1 comprising the amino acid sequence of SEQ ID NO: 4;
   (ii) an HVR-L2 comprising the amino acid sequence of SEQ ID NO: 5; and
   (iii) an HVR-L3 comprising the amino acid sequence of SEQ ID NO: 6; and
b) at least one antigen binding domain that specifically binds to CD3 comprising a heavy chain variable region comprising:
   (i) an HVR-H1 comprising the amino acid sequence of SEQ ID NO: 9;
   (ii) an HVR-H2 comprising the amino acid sequence of SEQ ID NO: 10; and
   (iii) an HVR-H3 comprising the amino acid sequence of SEQ ID NO: 11; and
a light chain variable region comprising:
   (i) an HVR-L1 comprising the amino acid sequence of SEQ ID NO: 12;
   (ii) an HVR-L2 comprising the amino acid sequence of SEQ ID NO: 13; and
   (iii) an HVR-L3 comprising the amino acid sequence of SEQ ID NO: 14.

In one embodiment, the anti-CD20/anti-CD3 bispecific antibody (e.g., anti-CD20/anti-CD3 TCB, e.g., glofitamab) comprises:
(i) at least one antigen binding domain that specifically binds to CD20 comprising the heavy chain variable region sequence of SEQ ID NO: 7 and the light chain variable region sequence of SEQ ID NO: 8, and
(ii) at least one antigen binding domain that specifically binds to CD3 comprising the heavy chain variable region sequence of SEQ ID NO: 15 and the light chain variable region sequence of SEQ ID NO: 16.

In one embodiment, the antigen binding domain that specifically binds to CD3 of the anti-CD20/anti-CD3 bispecific antibody is an antibody fragment, particularly a Fab molecule or a scFv molecule, more particularly a Fab molecule. In a particular embodiment, the antigen binding domain of the anti-CD20/anti-CD3 bispecific antibody (e.g., anti-CD20/anti-CD3 TCB, e.g., glofitamab) that specifically binds to CD3 is a crossover Fab molecule wherein the variable domains or the constant domains of the Fab heavy and light chain are exchanged (i.e., replaced by each other).

In one embodiment, the anti-CD20/anti-CD3 bispecific antibody (e.g., anti-CD20/anti-CD3 TCB, e.g., glofitamab) comprises at least one antigen binding domain that specifically binds to CD20, and one antigen binding domain that specifically binds to CD3. In one embodiment, the anti-CD20/anti-CD3 bispecific antibody (e.g., anti-CD20/anti-CD3 TCB, e.g., glofitamab) comprises a first antigen binding domain that specifically binds to CD3, and a second and a third antigen binding domain that specifically bind to CD20. In one embodiment, the first antigen binding domain is a crossover Fab molecule, and the second and the third antigen binding domain are each a conventional Fab molecule. In one embodiment, the anti-CD20/anti-CD3 bispecific antibody (e.g., anti-CD20/anti-CD3 TCB, e.g., glofitamab) further comprises an Fc domain. The anti-CD20/anti-CD3 bispecific antibody may comprise modifications in the Fc region and/or the antigen binding domains as described herein. In one embodiment, the anti-CD20/anti-CD3 bispecific antibody (e.g., anti-CD20/anti-CD3 TCB, e.g., glofitamab) comprises an IgG1 Fc domain comprising one or more amino acid substitutions that reduce binding to an Fc receptor and/or effector function. In one embodiment the anti-CD20/anti-CD3 bispecific antibody (e.g., anti-CD20/anti-CD3 TCB, e.g., glofitamab) comprises an IgG1 Fc domain comprising the amino acid substitutions L234A, L235A and P329G (EU numbering).

In one embodiment the anti-CD20/anti-CD3 bispecific antibody (e.g., anti-CD20/anti-CD3 TCB, e.g., glofitamab) comprises:
(i) an antigen binding domain that specifically binds to CD3 which is fused at the C-terminus of the Fab heavy chain to the N-terminus of the first subunit of the Fc domain,
(ii) a first antigen binding domain that specifically binds to CD20 which is fused at the C-terminus of the Fab heavy chain to the N-terminus of the Fab heavy chain of the antigen binding domain that specifically binds to CD3; and
(iii) a second antigen binding domain that specifically binds to CD20 which is fused at the C-terminus of the Fab heavy chain to the N-terminus of the second subunit of the Fc domain.

In a particular embodiment, the anti-CD20/anti-CD3 bispecific antibody (e.g., anti-CD20/anti-CD3 TCB, e.g., glofitamab) comprises:
a) a first Fab molecule which specifically binds to CD3, particularly CD3 epsilon; and wherein the variable domains VL and VH of the Fab light chain and the Fab heavy chain are replaced by each other;
b) a second Fab and a third Fab molecule which specifically bind to CD20, wherein in the constant domain CL of the second Fab and third Fab molecule the amino acid at position 124 is substituted by lysine (K) (numbering according to Kabat) and the amino acid at position 123 is substituted by lysine (K) or arginine (R), particularly by arginine (R) (numbering according to Kabat), and wherein in the constant domain CH1 o of the second Fab and third Fab molecule the amino acid at position 147 is substituted by glutamic acid (E) (EU numbering) and the amino acid at position 213 is substituted by glutamic acid (E) (EU numbering); and
c) a Fc domain composed of a first and a second subunit capable of stable association.

In one embodiment, the anti-CD20/anti-CD3 bispecific antibody (e.g., anti-CD20/anti-CD3 TCB, e.g., glofitamab) comprises two antigen binding domains that specifically bind to CD20 and one antigen binding domain that specifically binds to CD3.

In one embodiment, the anti-CD20/anti-CD3 bispecific antibody (e.g., anti-CD20/anti-CD3 TCB, e.g., glofitamab) is bivalent for CD20 and monovalent for CD3.

In one embodiment the first Fab molecule under a) is fused at the C-terminus of the Fab heavy chain to the N-terminus of one of the subunits of the Fc domain under c), the second Fab molecule under b) is fused at the C-terminus of the Fab heavy chain to the N-terminus of the heavy chain of the first Fab molecule under a), and the third Fab molecule under b) is fused at the C-terminus of the Fab heavy chain to the N-terminus of the other subunit of the Fc domain under c). In one embodiment, the first Fab molecule under a) comprises a heavy chain variable region that is at least 95%, 96%, 97%, 98%, or 99% identical to the sequence of SEQ ID NO: 15, and a light chain variable region that is at least 95%, 96%, 97%, 98%, or 99% identical to the sequence of SEQ ID NO: 16.

In still a further embodiment, the first Fab molecule under a) comprises the heavy chain variable region sequence of SEQ ID NO: 15, and the light chain variable region sequence of SEQ ID NO: 16.

In one embodiment, the second Fab molecule and the third Fab molecule under b) each comprise a heavy chain variable region that is at least 95%, 96%, 97%, 98%, or 99% identical to the sequence of SEQ ID NO: 7, and a light chain variable region that is at least 95%, 96%, 97%, 98%, or 99% identical to the sequence of SEQ ID NO: 8.

In one embodiment, the second Fab molecule under the third Fab molecule under b) each comprise the heavy chain variable region sequence of SEQ ID NO: 7, and the light chain variable region sequence of SEQ ID NO: 8.

In a particular embodiment, the anti-CD20/anti-CD3 bispecific antibody comprises a polypeptide that is at least 95%, 96%, 97%, 98%, or 99% identical to the sequence of SEQ ID NO: 17, a polypeptide that is at least 95%, 96%, 97%, 98%, or 99% identical to the sequence of SEQ ID NO: 18, a polypeptide that is at least 95%, 96%, 97%, 98%, or 99% identical to the sequence of SEQ ID NO: 19, and a polypeptide that is at least 95%, 96%, 97%, 98%, or 99% identical to the sequence of SEQ ID NO: 20. In a further particular embodiment, the bispecific antibody comprises a polypeptide sequence of SEQ ID NO: 17, a polypeptide sequence of SEQ ID NO: 18, a polypeptide sequence of SEQ ID NO: 19 and a polypeptide sequence of SEQ ID NO: 20. In a further particular embodiment, the bispecific antibody comprises one polypeptide chain comprising the amino acid sequence of SEQ ID NO: 17, one polypeptide chain comprising the amino acid sequence of SEQ ID NO: 18, one polypeptide chain comprising the amino acid sequence of SEQ ID NO: 19, and two polypeptide chains each comprising the amino acid sequence of SEQ ID NO: 20.

Particular anti-CD20/anti-CD3 bispecific antibodies are described in PCT publication no. WO 2016/020309 and European patent application nos. EP15188093 and EP16169160 (each incorporated herein by reference in its entirety). In one embodiment the anti-CD20/anti-CD3 bispecific antibody of the pharmaceutical composition of the invention is glofitamab.

### B. Antibody Formats

1. Configurations of the Anti-CD20/Anti-CD3 Bispecific Antibody

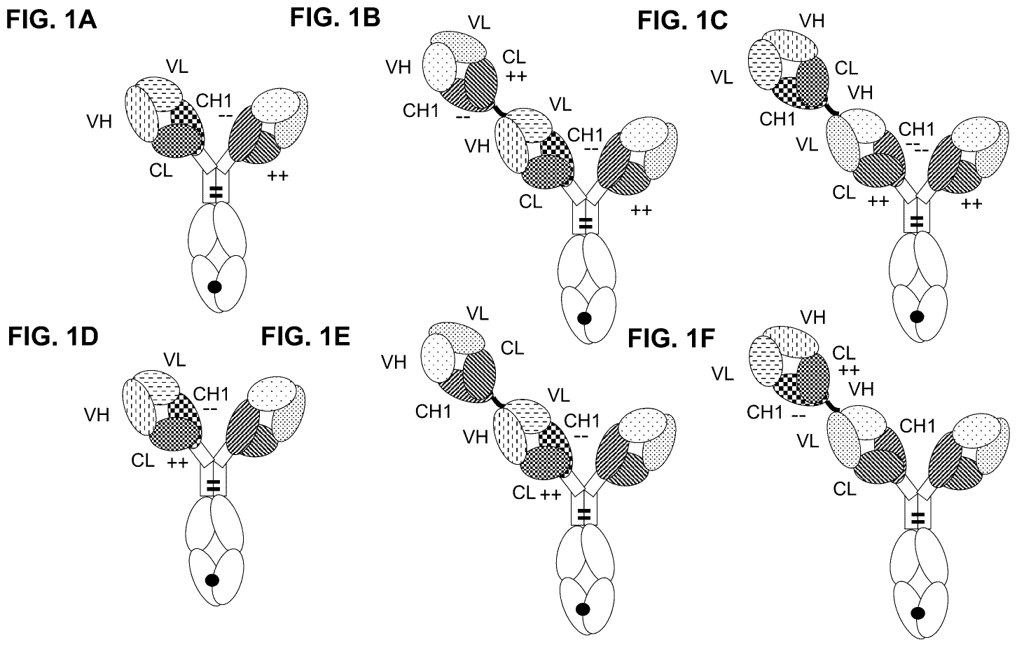

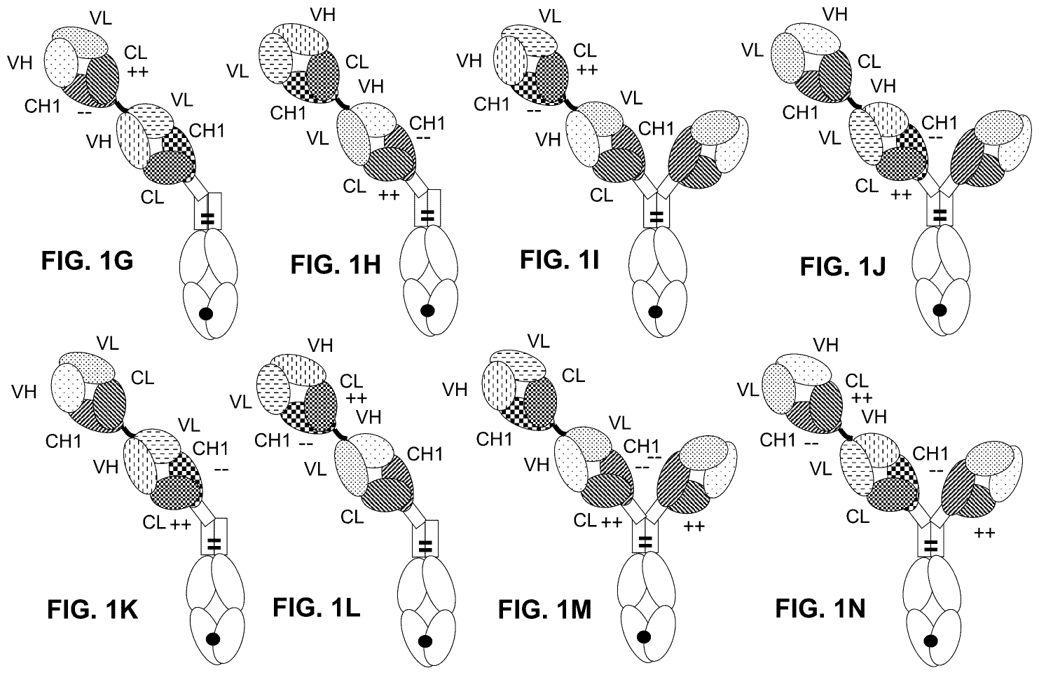

The components of the anti-CD20/anti-CD3 bispecific antibody can be fused to each other in a variety of configurations. Exemplary configurations are depicted in FIG. 1A-FIG. 1N.

In particular embodiments, the antigen binding moieties comprised in the anti-CD20/anti-CD3 bispecific antibody are Fab molecules. In such embodiments, the first, second, third, etc. antigen binding moiety may be referred to herein as first, second, third, etc. Fab molecule, respectively. Furthermore, in particular embodiments, the anti-CD20/anti-CD3 bispecific antibody comprises an Fc domain composed of a first and a second subunit capable of stable association.

In some embodiments, the first Fab molecule is fused at the C-terminus of the Fab heavy chain to the N-terminus of the first or the second subunit of the Fc domain.

In one such embodiment, the second Fab molecule is fused at the C-terminus of the Fab heavy chain to the N-terminus of the Fab heavy chain of the first Fab molecule. In a specific such embodiment, the anti-CD20/anti-CD3 bispecific antibody essentially consists of the first and the second Fab molecule, the Fc domain composed of a first and a second subunit, and optionally one or more peptide linkers, wherein the first Fab molecule is fused at the C-terminus of the Fab heavy chain to the N-terminus of the first or the second subunit of the Fc domain and the second Fab molecule is fused at the C-terminus of the Fab heavy chain to the N-terminus of the Fab heavy chain of the first Fab molecule. Such a configuration is schematically depicted in FIGS. 1G and 1K. Optionally, the Fab light chain of the first Fab molecule and the Fab light chain of the second Fab molecule may additionally be fused to each other.

In another embodiment, the second Fab molecule is fused at the C-terminus of the Fab heavy chain to the N-terminus of the first or second subunit of the Fc domain. In a specific such embodiment, the antibody essentially consists of the first and the second Fab molecule, the Fc domain composed of a first and a second subunit, and optionally one or more peptide linkers, wherein the first and the second Fab molecule are each fused at the C-terminus of the Fab heavy chain to the N-terminus of one of the subunits of the Fc domain. Such a configuration is schematically depicted in FIGS. 1A and 1D. The first and the second Fab molecule may be fused to the Fc domain directly or through a peptide linker. In a particular embodiment the first and the second Fab molecule are each fused to the Fc domain through an immunoglobulin hinge region. In a specific embodiment, the immunoglobulin hinge region is a human IgG₁ hinge region, particularly where the Fc domain is an IgG₁ Fc domain.

In other embodiments, the second Fab molecule is fused at the C-terminus of the Fab heavy chain to the N-terminus of the first or second subunit of the Fc domain. In one such embodiment, the first Fab molecule is fused at the C-terminus of the Fab heavy chain to the N-terminus of the Fab heavy chain of the second Fab molecule. In a specific such embodiment, the antibody essentially consists of the first and the second Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first or the second subunit of the Fc domain. Such a configuration is schematically depicted in FIGS. 1H and 1L. Optionally, the Fab light chain of the first Fab molecule and the Fab light chain of the second Fab molecule may additionally be fused to each other.

The Fab molecules may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ (SEQ ID NO: 21), (SG₄)ₙ (SEQ ID NO: 22), or G₄(SG₄)ₙ (SEQ ID NO: 23) peptide linkers. “n” is generally an integer from 1 to 10, typically from 2 to 4. In one embodiment said peptide linker has a length of at least 5 amino acids, in one embodiment a length of 5 to 100, in a further embodiment of 10 to 50 amino acids. In one embodiment said peptide linker is (GxS)ₙ or (GXS)ₙGₘ with G=glycine, S=serine, and (x=3, n=3, 4, 5 or 6, and m=0, 1, 2 or 3) or (x=4, n=2, 3, 4 or 5 and m=0, 1, 2 or 3) (SEQ ID NOs: 27-58), in one embodiment x=4 and n=2 or 3, in a further embodiment x=4 and n=2. In one embodiment said peptide linker is (G₄S)₂ (SEQ ID NO: 24). A particularly suitable peptide linker for fusing the Fab light chains of the first and the second Fab molecule to each other is (G₄S)₂ (SEQ ID NO: 24). An exemplary peptide linker suitable for connecting the Fab heavy chains of the first and the second Fab fragments comprises the sequence (D)-(G₄S)₂ (SEQ ID NOs: 24 and 25). Another suitable such linker comprises the sequence (G₄S)₄ (SEQ ID NO: 26). Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

An antibody with a single antigen binding moiety (such as a Fab molecule) capable of specific binding to a target cell antigen (for example as shown in FIG. 1A, 1D, 1G, 1H, 1K, or 1L) is useful, particularly in cases where internalization of the target cell antigen is to be expected following binding of a high affinity antigen binding moiety. In such cases, the presence of more than one antigen binding moiety specific for the target cell antigen may enhance internalization of the target cell antigen, thereby reducing its availability.

In many other cases, however, it will be advantageous to have an antibody comprising two or more antigen binding moieties (such as Fab molecules) specific for a target cell antigen (see examples shown in FIG. 1B, 1C, 1E, 1F, 1I, 1J, 1M, or 1N), for example to optimize targeting to the target site or to allow crosslinking of target cell antigens.

Accordingly, in particular embodiments, the anti-CD20/anti-CD3 bispecific antibody comprises two anti-CD20 binding moieties, e.g., two Fab molecules targeting CD20. In one embodiment the two Fab molecules targeting CD20 are conventional Fab molecules. In one embodiment, the two Fab molecules targeting CD20 comprise the same heavy and light chain amino acid sequences and have the same arrangement of domains (i.e., conventional or crossover).

In alternative embodiments, the anti-CD20/anti-CD3 bispecific antibody comprises two anti-CD3 binding moieties, e.g., two Fab molecules targeting CD3. In one such embodiment, the two Fab molecules targeting CD3 are both crossover Fab molecules (a Fab molecule wherein the variable domains VH and VL or the constant domains CL and CH1 of the Fab heavy and light chains are exchanged/replaced by each other). In one such embodiment, the two Fab molecules targeting CD3 comprise the same heavy and light chain amino acid sequences and have the same arrangement of domains (i.e., conventional or crossover).

In one embodiment, the third Fab molecule is fused at the C-terminus of the Fab heavy chain to the N-terminus of the first or second subunit of the Fc domain.

In a particular embodiment, the second and the third Fab molecule are each fused at the C-terminus of the Fab heavy chain to the N-terminus of one of the subunits of the Fc domain, and the first Fab molecule is fused at the C-terminus of the Fab heavy chain to the N-terminus of the Fab heavy chain of the second Fab molecule. In a specific such embodiment, the antibody essentially consists of the first, the second and the third Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first subunit of the Fc domain, and wherein the third Fab molecule is fused at the C-terminus of the Fab heavy chain to the N-terminus of the second subunit of the Fc domain. Such a configuration is schematically depicted in FIG. 1B and FIG. 1E (embodiments, wherein the third Fab molecule is a conventional Fab molecule and identical to the second Fab molecule), and FIG. 1I and FIG. 1M (embodiments, wherein the third Fab molecule is a crossover Fab molecule and preferably identical to the first Fab molecule). The second and the third Fab molecule may be fused to the Fc domain directly or through a peptide linker. In a particular embodiment the second and the third Fab molecule are each fused to the Fc domain through an immunoglobulin hinge region. In a specific embodiment, the immunoglobulin hinge region is a human IgG₁ hinge region, particularly where the Fc domain is an IgG₁ Fc domain. Optionally, the Fab light chain of the first Fab molecule and the Fab light chain of the second Fab molecule may additionally be fused to each other.

In another embodiment, the second and the third Fab molecule are each fused at the C-terminus of the Fab heavy chain to the N-terminus of one of the subunits of the Fc domain, and the first Fab molecule is fused at the C-terminus of the Fab heavy chain to the N-terminus of the Fab heavy chain of the second Fab molecule. In a specific such embodiment, the antibody essentially consists of the first, the second and the third Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first subunit of the Fc domain, and wherein the third Fab molecule is fused at the C-terminus of the Fab heavy chain to the N-terminus of the second subunit of the Fc domain. Such a configuration is schematically depicted in FIG. 1C and FIG. 1F (embodiments, wherein the third Fab molecule is a conventional Fab molecule and identical to the second Fab molecule) and in FIG. 1J and FIG. 1N (embodiments, wherein the third Fab molecule is a crossover Fab molecule and identical to the first Fab molecule). The first and the third Fab molecule may be fused to the Fc domain directly or through a peptide linker. In a particular embodiment the second and the third Fab molecule are each fused to the Fc domain through an immunoglobulin hinge region. In a specific embodiment, the immunoglobulin hinge region is a human IgG₁ hinge region, particularly where the Fc domain is an IgG₁ Fc domain. Optionally, the Fab light chain of the first Fab molecule and the Fab light chain of the second Fab molecule may additionally be fused to each other.

In configurations of the antibody wherein a Fab molecule is fused at the C-terminus of the Fab heavy chain to the N-terminus of each of the subunits of the Fc domain through an immunoglobulin hinge regions, the two Fab molecules, the hinge regions and the Fc domain essentially form an immunoglobulin molecule. In a particular embodiment, the immunoglobulin molecule is an IgG class immunoglobulin. In an even more particular embodiment, the immunoglobulin is an IgG₁ subclass immunoglobulin. In another embodiment, the immunoglobulin is an IgG₄ subclass immunoglobulin. In a further particular embodiment, the immunoglobulin is a human immunoglobulin. In other embodiments, the immunoglobulin is a chimeric immunoglobulin or a humanized immunoglobulin.

In some of the antibodies, the Fab light chain of the first Fab molecule and the Fab light chain of the second Fab molecule are fused to each other, optionally via a peptide linker. Depending on the configuration of the first and the second Fab molecule, the Fab light chain of the first Fab molecule may be fused at its C-terminus to the N-terminus of the Fab light chain of the second Fab molecule, or the Fab light chain of the second Fab molecule may be fused at its C-terminus to the N-terminus of the Fab light chain of the first Fab molecule. Fusion of the Fab light chains of the first and the second Fab molecule further reduces mispairing of unmatched Fab heavy and light chains, and also reduces the number of plasmids needed for expression of some of the antibodies.

In certain embodiments, the antibody comprises a polypeptide wherein the Fab light chain variable region of the first Fab molecule shares a carboxy-terminal peptide bond with the Fab heavy chain constant region of the first Fab molecule (i.e., the first Fab molecule comprises a crossover Fab heavy chain, wherein the heavy chain variable region is replaced by a light chain variable region), which in turn shares a carboxy-terminal peptide bond with an Fc domain subunit (VL₍₁₎-CH1₍₁₎-CH2-CH3(—CH4)), and a polypeptide wherein the Fab heavy chain of the second Fab molecule shares a carboxy-terminal peptide bond with an Fc domain subunit (VH₍₂₎-CH1₍₂₎-CH2-CH3(—CH4)). In some embodiments, the antibody further comprises a polypeptide wherein the Fab heavy chain variable region of the first Fab molecule shares a carboxy-terminal peptide bond with the Fab light chain constant region of the first Fab molecule (VH₍₁₎-CL₍₁₎) and the Fab light chain polypeptide of the second Fab molecule (VL₍₂₎-CL₍₂₎). In certain embodiments, the polypeptides are covalently linked, e.g., by a disulfide bond.

In certain embodiments, the antibody comprises a polypeptide wherein the Fab heavy chain variable region of the first Fab molecule shares a carboxy-terminal peptide bond with the Fab light chain constant region of the first Fab molecule (i.e., the first Fab molecule comprises a crossover Fab heavy chain, wherein the heavy chain constant region is replaced by a light chain constant region), which in turn shares a carboxy-terminal peptide bond with an Fc domain subunit (VH₍₁₎-CL₍₁₎-CH2-CH3(—CH4)), and a polypeptide wherein the Fab heavy chain of the second Fab molecule shares a carboxy-terminal peptide bond with an Fc domain subunit (VH₍₂₎-CH1₍₂₎-CH2-CH3(—CH4)). In some embodiments, the antibody further comprises a polypeptide wherein the Fab light chain variable region of the first Fab molecule shares a carboxy-terminal peptide bond with the Fab heavy chain constant region of the first Fab molecule (VL₍₁₎-CH1₍₁₎) and the Fab light chain polypeptide of the second Fab molecule (VL₍₂₎-CL₍₂₎). In certain embodiments the polypeptides are covalently linked, e.g., by a disulfide bond.

In some embodiments, the antibody comprises a polypeptide wherein the Fab light chain variable region of the first Fab molecule shares a carboxy-terminal peptide bond with the Fab heavy chain constant region of the first Fab molecule (i.e., the first Fab molecule comprises a crossover Fab heavy chain, wherein the heavy chain variable region is replaced by a light chain variable region), which in turn shares a carboxy-terminal peptide bond with the Fab heavy chain of the second Fab molecule, which in turn shares a carboxy-terminal peptide bond with an Fc domain subunit (VL₍₁₎-CH1₍₁₎-VH₍₂₎-CH1₍₂₎-CH2-CH3(—CH4)). In other embodiments, the antibody comprises a polypeptide wherein the Fab heavy chain of the second Fab molecule shares a carboxy-terminal peptide bond with the Fab light chain variable region of the first Fab molecule which in turn shares a carboxy-terminal peptide bond with the Fab heavy chain constant region of the first Fab molecule (i.e., the first Fab molecule comprises a crossover Fab heavy chain, wherein the heavy chain variable region is replaced by a light chain variable region), which in turn shares a carboxy-terminal peptide bond with an Fc domain subunit (VH₍₂₎-CH1₍₂₎-VL₍₁₎-CH1₍₁₎-CH2-CH3(—CH4)).

In some of these embodiments, the antibody further comprises a crossover Fab light chain polypeptide of the first Fab molecule, wherein the Fab heavy chain variable region of the first Fab molecule shares a carboxy-terminal peptide bond with the Fab light chain constant region of the first Fab molecule (VH₍₁₎-CL₍₁₎), and the Fab light chain polypeptide of the second Fab molecule (VL₍₂₎-CL₍₂₎). In others of these embodiments, the antibody further comprises a polypeptide wherein the Fab heavy chain variable region of the first Fab molecule shares a carboxy-terminal peptide bond with the Fab light chain constant region of the first Fab molecule which in turn shares a carboxy-terminal peptide bond with the Fab light chain polypeptide of the second Fab molecule (VH₍₁₎-CL₍₁₎-VL₍₂₎-CL₍₂₎), or a polypeptide wherein the Fab light chain polypeptide of the second Fab molecule shares a carboxy-terminal peptide bond with the Fab heavy chain variable region of the first Fab molecule which in turn shares a carboxy-terminal peptide bond with the Fab light chain constant region of the first Fab molecule (VL₍₂₎-CL₍₂₎-VH₍₁₎-CL₍₁₎), as appropriate.

The antibody according to these embodiments may further comprise (i) an Fc domain subunit polypeptide (CH2-CH3(—CH4)), or (ii) a polypeptide wherein the Fab heavy chain of a third Fab molecule shares a carboxy-terminal peptide bond with an Fc domain subunit (VH₍₃₎-CH1₍₃₎-CH2-CH3(—CH4)) and the Fab light chain polypeptide of a third Fab molecule (VL₍₃₎-CL₍₃₎). In certain embodiments the polypeptides are covalently linked, e.g., by a disulfide bond.

In some embodiments, the antibody comprises a polypeptide wherein the Fab heavy chain variable region of the second Fab molecule shares a carboxy-terminal peptide bond with the Fab light chain constant region of the first Fab molecule (i.e., the first Fab molecule comprises a crossover Fab heavy chain, wherein the heavy chain constant region is replaced by a light chain constant region), which in turn shares a carboxy-terminal peptide bond with the Fab heavy chain of the second Fab molecule, which in turn shares a carboxy-terminal peptide bond with an Fc domain subunit (VH₍₁₎-CL₍₁₎-VH₍₂₎-CH1₍₂₎-CH2-CH3(—CH4)). In other embodiments, the antibody comprises a polypeptide wherein the Fab heavy chain of the second Fab molecule shares a carboxy-terminal peptide bond with the Fab heavy chain variable region of the first Fab molecule which in turn shares a carboxy-terminal peptide bond with the Fab light chain constant region of the first Fab molecule (i.e., the first Fab molecule comprises a crossover Fab heavy chain, wherein the heavy chain constant region is replaced by a light chain constant region), which in turn shares a carboxy-terminal peptide bond with an Fc domain subunit (VH₍₂₎-CH1₍₂₎-VH₍₁₎-CL₍₁₎-CH2-CH3(—CH4)).

In some of these embodiments, the antibody further comprises a crossover Fab light chain polypeptide of the first Fab molecule, wherein the Fab light chain variable region of the first Fab molecule shares a carboxy-terminal peptide bond with the Fab heavy chain constant region of the first Fab molecule (VL₍₁₎-CH1₍₁₎), and the Fab light chain polypeptide of the second Fab molecule (VL₍₂₎-CL₍₂₎). In others of these embodiments, the antibody further comprises a polypeptide wherein the Fab light chain variable region of the first Fab molecule shares a carboxy-terminal peptide bond with the Fab heavy chain constant region of the first Fab molecule which in turn shares a carboxy-terminal peptide bond with the Fab light chain polypeptide of the second Fab molecule (VL₍₁₎-CH1₍₁₎-VL₍₂₎-CL₍₂₎), or a polypeptide wherein the Fab light chain polypeptide of the second Fab molecule shares a carboxy-terminal peptide bond with the Fab heavy chain variable region of the first Fab molecule which in turn shares a carboxy-terminal peptide bond with the Fab light chain constant region of the first Fab molecule (VL₍₂₎-CL₍₂₎-VH₍₁₎-CL₍₁₎), as appropriate.

The antibody according to these embodiments may further comprise (i) an Fc domain subunit polypeptide (CH2-CH3(—CH4)), or (ii) a polypeptide wherein the Fab heavy chain of a third Fab molecule shares a carboxy-terminal peptide bond with an Fc domain subunit (VH₍₃₎-CH1₍₃₎-CH2-CH3(—CH4)) and the Fab light chain polypeptide of a third Fab molecule (VL₍₃₎-CL₍₃₎). In certain embodiments, the polypeptides are covalently linked, e.g., by a disulfide bond.

In certain embodiments, the antibody comprises a polypeptide wherein the Fab heavy chain of the first Fab molecule shares a carboxy-terminal peptide bond with the Fab light chain variable region of the second Fab molecule, which in turn shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region) (VH₍₁₎-CH1₍₁₎-VL₍₂₎-CH1₍₂₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎).

In certain embodiments, the antibody comprises a polypeptide wherein the Fab light chain variable region of the second Fab molecule shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region), which in turn shares a carboxy-terminal peptide bond with the Fab heavy chain of the first Fab molecule (VL₍₂₎-CH1₍₂₎-VH₍₁₎-CH1₍₁₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎).

In certain embodiments, the antibody comprises a polypeptide wherein the Fab heavy chain variable region of the second Fab molecule shares a carboxy-terminal peptide bond with the Fab light chain constant region of the second Fab molecule (i.e., the second Fab molecule comprises a crossover Fab heavy chain, wherein the heavy chain constant region is replaced by a light chain constant region), which in turn shares a carboxy-terminal peptide bond with the Fab heavy chain of the first Fab molecule (VH₍₂₎-CL₍₂₎-VH₍₁₎-CH1₍₁₎). In some embodiments, the antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎).

In certain embodiments, the antibody comprises a polypeptide wherein the Fab heavy chain of a third Fab molecule shares a carboxy-terminal peptide bond with the Fab heavy chain of the first Fab molecule, which in turn shares a carboxy-terminal peptide bond with the Fab light chain variable region of the second Fab molecule, which in turn shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region) (VH₍₃₎-CH1₍₃₎-VH₍₁₎-CH1₍₁₎-VL₍₂₎-CH1₍₂₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments, the antibody further comprises the Fab light chain polypeptide of a third Fab molecule (VL₍₃₎-CL₍₃₎).

In certain embodiments, the antibody comprises a polypeptide wherein the Fab heavy chain of a third Fab molecule shares a carboxy-terminal peptide bond with the Fab heavy chain of the first Fab molecule, which in turn shares a carboxy-terminal peptide bond with the Fab heavy chain variable region of the second Fab molecule, which in turn shares a carboxy-terminal peptide bond with the Fab light chain constant region of the second Fab molecule (i.e., the second Fab molecule comprises a crossover Fab heavy chain, wherein the heavy chain constant region is replaced by a light chain constant region) (VH₍₃₎-CH1₍₃₎-VH₍₁₎-CH1₍₁₎-VH₍₂₎-CL₍₂₎). In some embodiments, the antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some embodiments, the antibody further comprises the Fab light chain polypeptide of a third Fab molecule (VL₍₃₎-CL₍₃₎).

In certain embodiments, the antibody comprises a polypeptide wherein the Fab light chain variable region of the second Fab molecule shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region), which in turn shares a carboxy-terminal peptide bond with the Fab heavy chain of the first Fab molecule, which in turn shares a carboxy-terminal peptide bond with the Fab heavy chain of a third Fab molecule (VL₍₂₎-CH1₍₂₎-VH₍₁₎-CH1₍₁₎-VH₍₃₎-CH1₍₃₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments, the antibody further comprises the Fab light chain polypeptide of a third Fab molecule (VL₍₃₎-CL₍₃₎).

In certain embodiments, the antibody comprises a polypeptide wherein the Fab heavy chain variable region of the second Fab molecule shares a carboxy-terminal peptide bond with the Fab light chain constant region of the second Fab molecule (i.e., the second Fab molecule comprises a crossover Fab heavy chain, wherein the heavy chain constant region is replaced by a light chain constant region), which in turn shares a carboxy-terminal peptide bond with the Fab heavy chain of the first Fab molecule, which in turn shares a carboxy-terminal peptide bond with the Fab heavy chain of a third Fab molecule (VH₍₂₎-CL₍₂₎-VH₍₁₎-CH1₍₁₎-VH₍₃₎-CH1₍₃₎). In some embodiments, the antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some embodiments, the antibody further comprises the Fab light chain polypeptide of a third Fab molecule (VL₍₃₎-CL₍₃₎).

In certain embodiments, the antibody comprises a polypeptide wherein the Fab heavy chain of the first Fab molecule shares a carboxy-terminal peptide bond with the Fab light chain variable region of the second Fab molecule, which in turn shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region), which in turn shares a carboxy-terminal peptide bond with the Fab light chain variable region of a third Fab molecule, which in turn shares a carboxy-terminal peptide bond with the Fab heavy chain constant region of a third Fab molecule (i.e., the third Fab molecule comprises a crossover Fab heavy chain, wherein the heavy chain variable region is replaced by a light chain variable region) (VH₍₁₎-CH1₍₁₎-VL₍₂₎-CH1₍₂₎-VL₍₃₎-CH1₍₃₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments, the antibody further comprises a polypeptide wherein the Fab heavy chain variable region of a third Fab molecule shares a carboxy-terminal peptide bond with the Fab light chain constant region of a third Fab molecule (VH₍₃₎-CL₍₃₎).

In certain embodiments, the antibody comprises a polypeptide wherein the Fab heavy chain of the first Fab molecule shares a carboxy-terminal peptide bond with the Fab heavy chain variable region of the second Fab molecule, which in turn shares a carboxy-terminal peptide bond with the Fab light chain constant region of the second Fab molecule (i.e., the second Fab molecule comprises a crossover Fab heavy chain, wherein the heavy chain constant region is replaced by a light chain constant region), which in turn shares a carboxy-terminal peptide bond with the Fab heavy chain variable region of a third Fab molecule, which in turn shares a carboxy-terminal peptide bond with the Fab light chain constant region of a third Fab molecule (i.e., the third Fab molecule comprises a crossover Fab heavy chain, wherein the heavy chain constant region is replaced by a light chain constant region) (VH₍₁₎-CH1₍₁₎-VH₍₂₎-CL₍₂₎-VH₍₃₎-CL₍₃₎). In some embodiments, the antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some embodiments, the antibody further comprises a polypeptide wherein the Fab light chain variable region of a third Fab molecule shares a carboxy-terminal peptide bond with the Fab heavy chain constant region of a third Fab molecule (VL₍₃₎-CH1₍₃₎).

In certain embodiments, the antibody comprises a polypeptide wherein the Fab light chain variable region of a third Fab molecule shares a carboxy-terminal peptide bond with the Fab heavy chain constant region of a third Fab molecule (i.e., the third Fab molecule comprises a crossover Fab heavy chain, wherein the heavy chain variable region is replaced by a light chain variable region), which in turn shares a carboxy-terminal peptide bond with the Fab light chain variable region of the second Fab molecule, which in turn shares a carboxy-terminal peptide bond with the Fab heavy chain constant region of the second Fab molecule (i.e., the second Fab molecule comprises a crossover Fab heavy chain, wherein the heavy chain variable region is replaced by a light chain variable region), which in turn shares a carboxy-terminal peptide bond with the Fab heavy chain of the first Fab molecule (VL₍₃₎-CH1₍₃₎-VL₍₂₎-CH1₍₂₎-VH₍₁₎-CH1₍₁₎). In some embodiments, the antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some embodiments, the antibody further comprises a polypeptide wherein the Fab heavy chain variable region of a third Fab molecule shares a carboxy-terminal peptide bond with the Fab light chain constant region of a third Fab molecule (VH₍₃₎-CL₍₃₎).

In certain embodiments, the antibody comprises a polypeptide wherein the Fab heavy chain variable region of a third Fab molecule shares a carboxy-terminal peptide bond with the Fab light chain constant region of a third Fab molecule (i.e., the third Fab molecule comprises a crossover Fab heavy chain, wherein the heavy chain constant region is replaced by a light chain constant region), which in turn shares a carboxy-terminal peptide bond with the Fab heavy chain variable region of the second Fab molecule, which in turn shares a carboxy-terminal peptide bond with the Fab light chain constant region of the second Fab molecule (i.e., the second Fab molecule comprises a crossover Fab heavy chain, wherein the heavy chain constant region is replaced by a light chain constant region), which in turn shares a carboxy-terminal peptide bond with the Fab heavy chain of the first Fab molecule (VH₍₃₎-CL₍₃₎-VH₍₂₎-CL₍₂₎-VH₍₁₎-CH1₍₁₎). In some embodiments, the antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some embodiments, the antibody further comprises a polypeptide wherein the Fab light chain variable region of a third Fab molecule shares a carboxy-terminal peptide bond with the Fab heavy chain constant region of a third Fab molecule (VL₍₃₎-CH1₍₃₎).

According to any of the above embodiments, components of the antibody (e.g., Fab molecules, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G₄S)ₙ (SEQ ID NO: 21), (SG4)ₙ (SEQ ID NO: 22), or G₄(SG₄)ₙ (SEQ ID NO: 23) peptide linkers, wherein n is generally an integer from 1 to 10, typically from 2 to 4.

2. Fc Domain

The anti-CD20/anti-CD3 bispecific antibody may comprise an Fc domain which consists of a pair of polypeptide chains comprising heavy chain domains of an antibody molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

In one embodiment, the Fc domain is an IgG Fc domain. In a particular embodiment, the Fc domain is an IgG₁ Fc domain. In another embodiment the Fc domain is an IgG4 Fc domain. In a more specific embodiment, the Fc domain is an IgG4 Fc domain comprising an amino acid substitution at position S228 (Kabat numbering), particularly the amino acid substitution S228P. This amino acid substitution reduces in vivo Fab arm exchange of IgG4 antibodies (see Stubenrauch et al., Drug Metabolism and Disposition 38, 84-91 (2010)). In a further particular embodiment, the Fc domain is human.

(i) Fc Domain Modifications Promoting Heterodimerization

The anti-CD20/anti-CD3 bispecific antibody may comprise different components (e.g., antigen binding domains) fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of such antibodies in recombinant production, it will thus be advantageous to introduce in the Fc domain of the antibody a modification promoting the association of the desired polypeptides.

Accordingly, in particular embodiments the Fc domain comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

Several approaches for modifications in the CH3 domain of the Fc domain in order to enforce heterodimerization are well described, e.g., in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed). These different approaches for improved heavy chain heterodimerization are contemplated as different alternatives in combination with heavy-light chain modifications (e.g., variable or constant region exchange/replacement in Fab arms, or introduction of substitutions of charged amino acids with opposite charges in the CH1/CL interface) which reduce light chain mispairing and Bence Jones-type side products.

In a specific embodiment said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g., in U.S. Pat. Nos. 5,731,168; 7,695,936; Ridgway et al., Prot Eng. 9, 617-621 (1996) and Carter, J Immunol Meth. 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in a particular embodiment, in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine (S), threonine (T), and valine (V).

The protuberance and cavity can be made by altering the nucleic acid encoding the polypeptides, e.g., by site-specific mutagenesis, or by peptide synthesis.

In a specific embodiment, in the CH3 domain of the first subunit of the Fc domain (the “knob” subunit) the threonine residue at position 366 is replaced with a tryptophan residue (T366W), and in the CH3 domain of the second subunit of the Fc domain (the “hole” subunit) the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A) (EU numbering).

In yet a further embodiment, in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C) (EU numbering). Introduction of these two cysteine residues results in formation of a disulfide bridge between the two subunits of the Fc domain, further stabilizing the dimer (Carter, J Immunol Methods 248, 7-15 (2001)).

In a particular embodiment, the first subunit of the Fc domain comprises amino acid substitutions S354C and T366W, and the second subunit of the Fc domain comprises amino acid substitutions Y349C, T366S, L368A and Y407V (EU numbering).

In a particular embodiment, the CD3 antigen binding moiety described herein is fused to the first subunit of the Fc domain (comprising the “knob” modification). Without wishing to be bound by theory, fusion of the CD3 antigen binding moiety to the knob-containing subunit of the Fc domain will (further) minimize the generation of bispecific antibodies comprising two CD3 antigen binding moieties (steric clash of two knob-containing polypeptides).

Other techniques of CH3-modification for enforcing the heterodimerization are contemplated as alternatives are described, e.g., in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one embodiment, the heterodimerization approach described in EP 1870459 A1, is used alternatively. This approach is based on the introduction of charged amino acids with opposite charges at specific amino acid positions in the CH3/CH3 domain interface between the two subunits of the Fc domain. One preferred embodiment are amino acid mutations R409D and K370E in one of the two CH3 domains (of the Fc domain) and amino acid mutations D399K and E357K in the other one of the CH3 domains of the Fc domain (EU numbering).

In another embodiment, the anti-CD20/anti-CD3 bispecific antibody may comprise amino acid mutation T366W in the CH3 domain of the first subunit of the Fc domain and amino acid mutations T366S, L368A, and Y407V in the CH3 domain of the second subunit of the Fc domain, and additionally amino acid mutations R409D and K370E in the CH3 domain of the first subunit of the Fc domain and amino acid mutations D399K and E357K in the CH3 domain of the second subunit of the Fc domain (EU numbering).

In another embodiment, the anti-CD20/anti-CD3 bispecific antibody may comprise amino acid mutations S354C and T366W in the CH3 domain of the first subunit of the Fc domain and amino acid mutations Y349C, T366S, L368A, and Y407V in the CH3 domain of the second subunit of the Fc domain, or the antibody comprises amino acid mutations Y349C and T366W in the CH3 domain of the first subunit of the Fc domain and amino acid mutations S354C, T366S, L368A, and Y407V in the CH3 domains of the second subunit of the Fc domain and additionally amino acid mutations R409D and K370E in the CH3 domain of the first subunit of the Fc domain and amino acid mutations D399K and E357K in the CH3 domain of the second subunit of the Fc domain (all EU numbering).

In one embodiment, the heterodimerization approach described in WO 2013/157953 is used alternatively. In one embodiment a first CH3 domain comprises amino acid mutation T366K and a second CH3 domain comprises amino acid mutation L351 D (EU numbering). In a further embodiment, the first CH3 domain comprises further amino acid mutation L351K. In a further embodiment, the second CH3 domain comprises further an amino acid mutation selected from Y349E, Y349D, and L368E (preferably L368E) (EU numbering).

In one embodiment, the heterodimerization approach described in WO 2012/058768 is used alternatively. In one embodiment, a first CH3 domain comprises amino acid mutations L351Y, Y407A, and a second CH3 domain comprises amino acid mutations T366A and K409F. In a further embodiment, the second CH3 domain comprises a further amino acid mutation at position T411, D399, S400, F405, N390, or K392, e.g., selected from (a) T411N, T411R, T411Q, T411K, T411D, T411E, or T411W; (b) D399R, D399W, D399Y, or D399K; (c) S400E, S400D, S400R, or S400K; (d) F405I, F405M, F405T, F405S, F405V, or F405W; (e) N390R, N390K, or N390D; or (f) K392V, K392M, K392R, K392L, K392F, or K392E (EU numbering). In a further embodiment, a first CH3 domain comprises amino acid mutations L351Y and Y407A and a second CH3 domain comprises amino acid mutations T366V and K409F. In a further embodiment, a first CH3 domain comprises amino acid mutation Y407A and a second CH3 domain comprises amino acid mutations T366A and K409F. In a further embodiment, the second CH3 domain further comprises amino acid mutations K392E, T411E, D399R, and S400R (EU numbering).

In one embodiment, the heterodimerization approach described in WO 2011/143545 is used alternatively, e.g., with the amino acid modification at a position selected from the group consisting of 368 and 409 (EU numbering).

In one embodiment, the heterodimerization approach described in WO 2011/090762, which also uses the knobs-into-holes technology described above, is used alternatively. In one embodiment, a first CH3 domain comprises amino acid mutation T366W and a second CH3 domain comprises amino acid mutation Y407A. In one embodiment, a first CH3 domain comprises amino acid mutation T366Y and a second CH3 domain comprises amino acid mutation Y407T (EU numbering).

In one embodiment, the anti-CD20/anti-CD3 bispecific antibody or the Fc domain of the anti-CD20/anti-CD3 bispecific antibody is of IgG₂ subclass and the heterodimerization approach described in WO 2010/129304 is used.

In an alternative embodiment, a modification promoting association of the first and the second subunit of the Fc domain comprises a modification mediating electrostatic steering effects, e.g., as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such embodiment, a first CH3 domain comprises amino acid substitution of K392 or N392 with a negatively charged amino acid (e.g., glutamic acid (E), or aspartic acid (D), preferably K392D or N392D) and a second CH3 domain comprises amino acid substitution of D399, E356, D356, or E357 with a positively charged amino acid (e.g., lysine (K) or arginine (R), preferably D399K, E356K, D356K, or E357K, and more preferably D399K and E356K). In a further embodiment the first CH3 domain further comprises amino acid substitution of K409 or R409 with a negatively charged amino acid (e.g., glutamic acid (E), or aspartic acid (D), preferably K409D or R409D).

In a further embodiment the first CH3 domain further or alternatively comprises amino acid substitution of K439 and/or K370 with a negatively charged amino acid (e.g., glutamic acid (E), or aspartic acid (D)) (EU numbering).

In yet a further embodiment, the heterodimerization approach described in WO 2007/147901 is used alternatively. In one embodiment, a first CH3 domain comprises amino acid mutations K253E, D282K, and K322D and a second CH3 domain comprises amino acid mutations D239K, E240K, and K292D (EU numbering).

In still another embodiment, the heterodimerization approach described in WO 2007/110205 can be used.

In one embodiment, the first subunit of the Fc domain comprises amino acid substitutions K392D and K409D, and the second subunit of the Fc domain comprises amino acid substitutions D356K and D399K (EU numbering).

(ii) Fc Domain Modifications Reducing Fc Receptor Binding and/or Effector Function

The Fc domain confers to an antibody, such as an anti-CD20/anti-CD3 bispecific, favorable pharmacokinetic properties, including a long serum half-life which contributes to good accumulation in the target tissue and a favorable tissue-blood distribution ratio. At the same time, however, it may lead to undesirable targeting of the antibody to cells expressing Fc receptors rather than to the preferred antigen-bearing cells. Moreover, the co-activation of Fc receptor signaling pathways may lead to cytokine release which, in combination with other immunostimulatory properties the antibody may have and the long half-life of the antibody, results in excessive activation of cytokine receptors and severe side effects upon systemic administration.

Accordingly, in particular embodiments, the Fc domain of the anti-CD20/anti-CD3 bispecific antibody exhibits reduced binding affinity to an Fc receptor and/or reduced effector function, as compared to a native IgG₁ Fc domain. In one such embodiment, the Fc domain (or the molecule, e.g., antibody, comprising said Fc domain) exhibits less than 50%, preferably less than 20%, more preferably less than 10% and most preferably less than 5% of the binding affinity to an Fc receptor, as compared to a native IgG₁ Fc domain (or a corresponding molecule comprising a native IgG₁ Fc domain), and/or less than 50%, preferably less than 20%, more preferably less than 10% and most preferably less than 5% of the effector function, as compared to a native IgG₁ Fc domain (or a corresponding molecule comprising a native IgG₁ Fc domain). In one embodiment, the Fc domain (or the molecule, e.g., antibody, comprising said Fc domain) does not substantially bind to an Fc receptor and/or induce effector function. In a particular embodiment, the Fc receptor is an Fcγ receptor. In one embodiment the Fc receptor is a human Fc receptor. In one embodiment, the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment, the effector function is one or more selected from the group of CDC, ADCC, ADCP, and cytokine secretion. In a particular embodiment, the effector function is ADCC. In one embodiment the Fc domain exhibits substantially similar binding affinity to neonatal Fc receptor (FcRn), as compared to a native IgG₁ Fc domain. Substantially similar binding to FcRn is achieved when the Fc domain (or the molecule, e.g., antibody, comprising said Fc domain) exhibits greater than about 70%, particularly greater than about 80%, more particularly greater than about 90% of the binding affinity of a native IgG₁ Fc domain (or the corresponding molecule comprising a native IgG₁ Fc domain) to FcRn.

In certain embodiments, the Fc domain is engineered to have reduced binding affinity to an Fc receptor and/or reduced effector function, as compared to a non-engineered Fc domain. In particular embodiments, the Fc domain comprises one or more amino acid mutation that reduces the binding affinity of the Fc domain to an Fc receptor and/or effector function. Typically, the same one or more amino acid mutation is present in each of the two subunits of the Fc domain. In one embodiment, the amino acid mutation reduces the binding affinity of the Fc domain to an Fc receptor. In one embodiment, the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment, the molecule, e.g., antibody, comprising an engineered Fc domain exhibits less than 20%, particularly less than 10%, more particularly less than 5% of the binding affinity to an Fc receptor as compared to a corresponding molecule comprising a non-engineered Fc domain. In a particular embodiment, the Fc receptor is an Fcγ receptor. In some embodiments, the Fc receptor is a human Fc receptor. In some embodiments, the Fc receptor is an activating Fc receptor. In a specific embodiment, the Fc receptor is an activating human Fcγ receptor, more specifically human FcγRIIIa, FcγRI or FcγRIIa, most specifically human FcγRIIIa. Preferably, binding to each of these receptors is reduced. In some embodiments, binding affinity to a complement component, specifically binding affinity to C1q, is also reduced. In one embodiment, binding affinity to neonatal Fc receptor (FcRn) is not reduced. Substantially similar binding to FcRn, i.e., preservation of the binding affinity of the Fc domain to said receptor, is achieved when the Fc domain (or the molecule, e.g., antibody, comprising said Fc domain) exhibits greater than about 70% of the binding affinity of a non-engineered form of the Fc domain (or a corresponding molecule comprising said non-engineered form of the Fc domain) to FcRn. The Fc domain, or molecule (e.g., antibody) comprising said Fc domain, may exhibit greater than about 80% and even greater than about 90% of such affinity. In certain embodiments, the Fc domain is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment, the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or a corresponding molecule comprising a non-engineered Fc domain).

In one embodiment, the amino acid mutation that reduces the binding affinity of the Fc domain to an Fc receptor and/or effector function is an amino acid substitution. In one embodiment the Fc domain comprises an amino acid substitution at a position selected from the group of E233, L234, L235, N297, P331, and P329 (EU numbering). In a more specific embodiment, the Fc domain comprises an amino acid substitution at a position selected from the group of L234, L235, and P329 (EU numbering). In some embodiments, the Fc domain comprises the amino acid substitutions L234A and L235A (EU numbering).

In one such embodiment, the Fc domain is an IgG₁ Fc domain, particularly a human IgG₁ Fc domain. In one embodiment, the Fc domain comprises an amino acid substitution at position P329. In a more specific embodiment, the amino acid substitution is P329A or P329G, particularly P329G (EU numbering). In one embodiment, the Fc domain comprises an amino acid substitution at position P329 and a further amino acid substitution at a position selected from E233, L234, L235, N297, and P331 (EU numbering). In a more specific embodiment, the further amino acid substitution is E233P, L234A, L235A, L235E, N297A, N297D, or P331S. In particular embodiments, the Fc domain comprises amino acid substitutions at positions P329, L234, and L235 (EU numbering). In more particular embodiments, the Fc domain comprises the amino acid mutations L234A, L235A, and P329G (“P329G LALA”). In one such embodiment, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, incorporated herein by reference in its entirety. WO 2012/130831 also describes methods of preparing such mutant Fc domains and methods for determining its properties such as Fc receptor binding or effector functions.

IgG4 antibodies exhibit reduced binding affinity to Fc receptors and reduced effector functions as compared to IgG₁ antibodies. Hence, in some embodiments, the Fc domain is an IgG4 Fc domain, particularly a human IgG4 Fc domain. In one embodiment, the IgG4 Fc domain comprises amino acid substitutions at position S228, specifically the amino acid substitution S228P (EU numbering). To further reduce its binding affinity to an Fc receptor and/or its effector function, in one embodiment the IgG4 Fc domain comprises an amino acid substitution at position L235, specifically the amino acid substitution L235E (EU numbering). In another embodiment, the IgG4 Fc domain comprises an amino acid substitution at position P329, specifically the amino acid substitution P329G (EU numbering). In a particular embodiment, the IgG4 Fc domain comprises amino acid substitutions at positions S228, L235, and P329, specifically amino acid substitutions S228P, L235E, and P329G (EU numbering). Such IgG4 Fc domain mutants and their Fcγ receptor binding properties are described in PCT Publication No. WO 2012/130831, incorporated herein by reference in its entirety.

In a particular embodiment, the Fc domain exhibiting reduced binding affinity to an Fc receptor and/or reduced effector function, as compared to a native IgG₁ Fc domain, is a human IgG₁ Fc domain comprising the amino acid substitutions L234A, L235A, and optionally P329G, or a human IgG4 Fc domain comprising the amino acid substitutions S228P, L235E, and optionally P329G (EU numbering).

In certain embodiments, N-glycosylation of the Fc domain has been eliminated. In one such embodiment, the Fc domain comprises an amino acid mutation at position N297, particularly an amino acid substitution replacing asparagine by alanine (N297A) or aspartic acid (N297D) or glycine (N297G) (EU numbering).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327, and 329 (U.S. Pat. No. 6,737,056) (EU numbering). Such Fc mutants include Fc mutants with substitutions at two or more of amino acid positions 265, 269, 270, 297, and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined, e.g., by ELISA, or by Surface Plasmon Resonance (SPR) using standard instrumentation such as a BIACORE® instrument (GE Healthcare), and Fc receptors such as may be obtained by recombinant expression. Alternatively, binding affinity of Fc domains or molecules comprising an Fc domain for Fc receptors may be evaluated using cell lines known to express particular Fc receptors, such as human NK cells expressing FcγIIIa receptor.

Effector function of an Fc domain, or a molecule (e.g., an antibody) comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA. 83, 7059-7063 (1986) and Hellstrom et al., Proc Natl Acad Sci USA. 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CYTOTOX 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed i, e.g., in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the Fc domain, or molecule (e.g., antibody) comprising the Fc domain, is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

3. Substitution, Insertion, and Deletion

In certain instances, the anti-CD20/anti-CD3 bispecific antibody variants of the pharmaceutical compositions provided herein have one or more amino acid substitutions. Sites of interest for substitutional mutagenesis include the HVRs and FRs. Conservative substitutions are shown in Table 3 under the heading of “preferred substitutions.” More substantial changes are provided in Table 3 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 3
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al., in Methods in Molecular Biology 178:1-37 (O'Brien et al., ed., Human Press, Totowa, NJ, (2001)). In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more HVRs so long as such alterations do not substantially reduce the ability of the antibody to bind antigen. For example, conservative alterations (e.g., conservative substitutions as described herein) that do not substantially reduce binding affinity may be made in HVRs. Such alterations may, for example, be outside of antigen contacting residues in the HVRs. In certain instances of the variant VH and VL sequences described above, each HVR either is unaltered, or includes no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

4. Glycosylation

In certain instances, anti-CD20/anti-CD3 bispecific antibodies comprised in the pharmaceutical compositions of the invention can be altered to increase or decrease the extent to which the antibody is glycosylated. Addition or deletion of glycosylation sites to anti-CD20/anti-CD3 bispecific antibodies may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody are made in order to create antibody variants with certain improved properties.

In one instance, anti-CD20/anti-CD3 bispecific antibody variants have a carbohydrate structure that lacks fucose attached (directly or indirectly) to an Fc region. For example, the amount of fucose in such antibody may be from 1% to 80%, from 1% to 65%, from 5% to 65%, or from 20% to 40%. The amount of fucose is determined by calculating the average amount of fucose within the sugar chain at Asn297, relative to the sum of all glycostructures attached to Asn297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., U.S. Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al., J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545 (1986); U.S. Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al., Biotech. Bioeng. 87: 614 (2004); Kanda, Y. et al., Biotechnol. Bioeng., 94(4):680-688 (2006); and WO 2003/085107).

In view of the above, in some instances, the pharmaceutical compositions of the invention comprise an anti-CD20/anti-CD3 bispecific antibody variant that comprises an aglycosylation site mutation. In some instances, the aglycosylation site mutation reduces effector function of the antibody. In some instances, the aglycosylation site mutation is a substitution mutation. In some instances, the antibody comprises a substitution mutation in the Fc region that reduces effector function. In some instances, the substitution mutation is at amino acid residue N297, L234, L235, and/or D265 (EU numbering). In some instances, the substitution mutation is selected from the group consisting of N297G, N297A, L234A, L235A, D265A, and P329G. In some instances, the substitution mutation is at amino acid residue N297. In a preferred instance, the substitution mutation is N297A.

Anti-CD20/anti-CD3 bispecific antibody variants may comprise bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and U.S. 2005/0123546. Other antibody variants comprise at least one galactose residue in the oligosaccharide attached to the Fc region. Such antibody variants may have improved CDC function. Such antibody variants are described, e.g., in WO 1997/30087, WO 1998/58964, and WO 1999/22764.

5. Antibody Derivatives

In certain instances, an anti-CD20/anti-CD3 bispecific antibody of the pharmaceutical compositions provided herein is further modified to contain additional nonproteinaceous moieties that are known in the art and readily available. The moieties suitable for derivatization of the antibody include, but are not limited to, water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another instance, conjugates of an antibody and nonproteinaceous moiety may be selectively heated by exposure to radiation. In one instance, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### C. Recombinant Production Methods

Anti-CD20/anti-CD3 bispecific antibodies (e.g., anti-CD20/anti-CD3 TCBs, e.g., glofitamab) of the pharmaceutical compositions of the invention may be produced using recombinant methods and compositions, for example, as described in U.S. Pat. No. 4,816,567, which is incorporated herein by reference in its entirety.

For recombinant production of an anti-CD20/anti-CD3 bispecific antibody, nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR− CHO cells (Urlaub et al., Proc. Natl. Acad. Sci. USA 77:4216 (1980)); and myeloma cell lines such as Y0, NS0, and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### V. Therapeutic Methods and Uses

The pharmaceutical compositions comprising an anti-CD20/anti-CD3 bispecific antibody described herein can be formulated for use as a medicament for treating various diseases and disorders. Thus, the invention features methods involving intravenous administration of the pharmaceutical composition to a subject in need thereof, e.g., a subject having a disease or disorder, such as cancer. A pharmaceutical composition of the present invention may be used to treat or delay progression of a cell proliferative disorder in a subject in need thereof (e.g., a human subject in need thereof) or to enhance immune function in a subject having a cell proliferative disorder (e.g., cancer).

In one aspect, the invention provides a pharmaceutical composition as described herein for use in treating or delaying progression of a cell proliferative disorder. In one aspect, the invention provides the use of a pharmaceutical composition as described herein in the manufacture of a medicament for treating or delaying progression of a cell proliferative disorder. In one aspect, the invention provides a method of treating or delaying progression of a cell proliferative disorder in a subject in need thereof, comprising administering to the subject a pharmaceutical composition as described herein.

In some embodiments, the cell proliferative disorder is a cancer that is a non-Hodgkin's lymphoma (NHL). In some embodiments, the NHL is selected from the group consisting of non-Hodgkin's lymphoma (NHL), chronic lymphoid leukemia (CLL), B cell lymphoma, splenic diffuse red pulp small B cell lymphoma, B cell lymphoma with features intermediate between diffuse large B-cell lymphoma and Burkitt lymphoma, Burkitt-like lymphoma with 11q aberration, B cell lymphoma with features intermediate between diffuse large B-cell lymphoma and classical Hodgkin lymphoma, germinal center B cell-like (GCB) diffuse large B cell lymphoma (DLBCL), activated B cell-like (ABC) DLBCL, primary cutaneous follicle centrecenter lymphoma, T-cell/histiocyte-rich large B-cell lymphoma, primary DLBCL of the central nervous system, primary cutaneous DLBCL (leg type), Epstein-Barr virus (EBV)-positive DLBCL of the elderly, DLBCL associated with chronic inflammation, primary mediastinal (thymic) large B cell lymphoma, intravascular large B cell lymphoma, ALK-positive large B-cell lymphoma, large B-cell lymphoma arising in HHV8-associated multicentric Castleman disease, B cell leukemia, breast cancer, colorectal cancer, non-small cell lung cancer, multiple myeloma, renal cancer, prostate cancer, liver cancer, head and neck cancer, melanoma, ovarian cancer, mesothelioma, glioblastoma, follicular lymphoma (FL), in situ follicular neoplasia, mantle cell lymphoma (MCL), in situ mantle cell neoplasia, acute myeloid leukemia (AML), marginal zone lymphoma (MZL), small lymphocytic leukemia (SLL), lymphoplasmacytic lymphoma (LL), central nervous system lymphoma (CNSL), Burkitt's lymphoma (BL), B cell prolymphocytic leukemia, splenic marginal zone lymphoma, hairy cell leukemia, splenic lymphoma/leukemia, hairy cell leukemia variant, a heavy chain disease, γ heavy chain disease, p heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, lymphomatoid granulomatosis, plasmablastic lymphoma, and primary effusion lymphoma. In particular embodiments, the cancer is germinal center B cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), or Burkitt's lymphoma (BL).

In some embodiments, the cancer is selected from the group consisting of breast cancer, colorectal cancer, non-small cell lung cancer (NSCLC), multiple myeloma, renal cancer, prostate cancer, liver cancer, head and neck cancer, melanoma, ovarian cancer, mesothelioma, and glioblastoma.

The anti-CD20/anti-CD3 bispecific antibody can be formulated for administration to the subject at a dosage of 0.5 mg, 2.5 mg, 10 mg, or 30 mg.

For all the methods and pharmaceutical formulations described herein, the anti-CD20/anti-CD3 bispecific antibody (e.g., anti-CD20/anti-CD3 TCB, e.g., glofitamab) would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The anti-CD20/anti-CD3 bispecific antibody (e.g., anti-CD20/anti-CD3 TCB, e.g., glofitamab) need not be, but is optionally formulated with, one or more agents currently used to prevent or treat the disorder in question. The effective amount of such other agents depends on the amount of the anti-CD20/anti-CD3 bispecific antibody (e.g., anti-CD20/anti-CD3 TCB, e.g., glofitamab) present in the formulation, the type of disorder or treatment, and other factors discussed above. The anti-CD20/anti-CD3 bispecific antibody (e.g., anti-CD20/anti-CD3 TCB, e.g., glofitamab) may be suitably administered to the patient over a series of treatments.

### VI. Articles of Manufacture

In another aspect of the invention, an article of manufacture containing materials useful for the treatment, prevention, and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a pharmaceutical composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an anti-CD20/anti-CD3 bispecific antibody (e.g., anti-CD20/anti-CD3 TCB, e.g., glofitamab), as described herein. The label or package insert indicates that the composition is used for treating the condition of choice (e.g., a cancer) and further includes information related to at least one of the dosing regimens described herein.

The pharmaceutical composition can be supplied in a container having a volume from 1 ml to 100 ml (e.g., from 1 ml to 5 ml, from 5 ml to 10 ml, from 10 ml to 15 ml, from 15 ml to 20 ml, from 20 ml to 25 ml, from 25 ml to 30 ml, from 30 ml to 40 ml, from 40 ml to 50 ml, from 50 ml to 60 ml, from 60 ml to 70 ml, from 70 ml to 80 ml, from 80 ml to 90 ml, or from 90 ml to 100 ml, e.g., about 5 ml, about 10 ml, about 15 ml, about 20 ml, about 25 ml, about 30 ml, about 40 ml, about 50 ml, about 60 ml, about 70 ml, about 80 ml, about 90 ml, or about 100 ml).

In some embodiments, the container is a stainless steel container or a nickel-steel alloy container (e.g., HASTELLOY®), such as a tank, mini-tank, canister, can, etc. In some instances, the pharmaceutical composition in such a container is a drug substance (DS), which can be further diluted prior to use, e.g., into a drug product (DP) (e.g., in final vial configuration). Alternatively, the pharmaceutical composition in the container is a DP. In some embodiments, the DP is in a container such as an IV bag or a syringe (e.g., for delivery via syringe pump).

In some embodiments, the article of manufacture includes a vial having a volume of about 1 ml or more, for example, about 1 ml, about 2 ml, about 3 ml, about 4 ml, about 5 ml, about 6 ml, about 7 ml, about 8 ml, about 9 ml, about 10 ml, about 11 ml, about 12 ml, about 13 ml, about 14 ml, about 15 ml, about 16 ml, about 17 ml, about 18 ml, about 19 ml, about 20 ml, about 25 ml, about 30 ml, about 35 ml, about 40 ml, about 50 ml, or more. In some embodiments, the container is a vial having a volume of about 10 ml. In some embodiments, the vial is for single-use. In some embodiments, the vial contains about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, or more of the anti-CD20/anti-CD3 bispecific antibody (e.g., anti-CD20/anti-CD3 TCB, e.g., glofitamab). In some embodiments, the container closure system comprises one or more, or all, of a glass vial, a stopper, and a cap.

Moreover, the article of manufacture may comprise (a) a first container with a composition contained therein, wherein the pharmaceutical composition comprises an anti-CD20/anti-CD3 bispecific antibody (e.g., anti-CD20/anti-CD3 TCB, e.g., glofitamab) described herein; and (b) a second container with a pharmaceutical composition contained therein, wherein the pharmaceutical composition comprises a further cytotoxic or otherwise therapeutic agent. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

A further aspect of the present invention relates to the invention as described hereinbefore.

### EMBODIMENTS

Some embodiments of the technology described herein can be defined according to any of the following numbered embodiments:
I. A liquid pharmaceutical composition comprising:
   about 1 to 25 mg/ml of an anti-CD20/anti-CD3 bispecific antibody;
   about 10 to 50 mM of a buffering agent;
   about ≥200 mM of a tonicity agent;
   about 0-15 mM methionine; and
   about 0.2 mg/ml of a surfactant
   at a pH in the range of from about 5.0 to about 6.0,
wherein the anti-CD20/anti-CD3 bispecific antibody comprises:
a) at least one antigen binding domain that specifically binds to CD20 comprising
   a heavy chain variable region comprising:
      (i) an HVR-H1 comprising the amino acid sequence of SEQ ID NO: 1;
      (ii) an HVR-H2 comprising the amino acid sequence of SEQ ID NO: 2; and
      (iii) an HVR-H3 comprising the amino acid sequence of SEQ ID NO:3;
   and a light chain variable region comprising:
      (i) an HVR-L1 comprising the amino acid sequence of SEQ ID NO: 4;
      (ii) an HVR-L2 comprising the amino acid sequence of SEQ ID NO: 5; and
      (iii) an HVR-L3 comprising the amino acid sequence of SEQ ID NO: 6; and
b) at least one antigen binding domain that specifically binds to CD3 comprising
   a heavy chain variable region comprising:
      (i) an HVR-H1 comprising the amino acid sequence of SEQ ID NO: 9;
      (ii) an HVR-H2 comprising the amino acid sequence of SEQ ID NO: 10; and
      (iii) an HVR-H3 comprising the amino acid sequence of SEQ ID NO:11; and
   a light chain variable region comprising:
      (i) an HVR-L1 comprising the amino acid sequence of SEQ ID NO: 12;
      (ii) an HVR-L2 comprising the amino acid sequence of SEQ ID NO: 13; and
      (iii) an HVR-L3 comprising the amino acid sequence of SEQ ID NO: 14.
II. The liquid pharmaceutical composition according to embodiment I, wherein the anti-CD20/anti-CD3 bispecific antibody concentration is in the range of about 1 to 5 mg/ml.
III. The liquid pharmaceutical composition according to any one of the preceding embodiments, wherein the anti-CD20/anti-CD3 bispecific antibody concentration is in the range of about 0.9-1.1 mg/ml.
IV. The liquid pharmaceutical composition according to any one of the preceding embodiments, wherein the anti-CD20/anti-CD3 bispecific antibody concentration is about 1 mg/ml.
V. The liquid pharmaceutical composition according to any one of the preceding embodiments, wherein the anti-CD20/anti-CD3 bispecific antibody comprises:
   a) at least one antigen binding domain that specifically binds to CD20 comprising the heavy chain variable region sequence of SEQ ID NO: 7 and the light chain variable region sequence of SEQ ID NO: 8, and
   b) at least one antigen binding domain that specifically binds to CD3 comprising the heavy chain variable region sequence of SEQ ID NO: 15 and the light chain variable region sequence of SEQ ID NO: 16.
VI. The liquid pharmaceutical composition according to any one of the preceding embodiments, wherein the anti-CD20/anti-CD3 bispecific antibody comprises:
   a) a first Fab molecule which specifically binds to CD3, particularly CD3 epsilon; and wherein the variable domains VL and VH of the Fab light chain and the Fab heavy chain are replaced by each other;
   b) a second Fab and a third Fab molecule which specifically bind to CD20, wherein in the constant domain CL of the second Fab and third Fab molecule the amino acid at position 124 is substituted by lysine (K) (numbering according to Kabat) and the amino acid at position 123 is substituted by lysine (K) or arginine (R), particularly by arginine (R) (numbering according to Kabat), and wherein in the constant domain CH1 o of the second Fab and third Fab molecule the amino acid at position 147 is substituted by glutamic acid (E) (EU numbering) and the amino acid at position 213 is substituted by glutamic acid (E) (EU numbering); and
   c) c) a Fc domain composed of a first and a second subunit capable of stable association.
VII. The liquid pharmaceutical composition according to any one of the preceding embodiments, wherein the anti-CD20/anti-CD3 bispecific antibody is glofitamab.
VIII. The liquid pharmaceutical composition according to any one of the preceding embodiments, wherein the buffering agent is a histidine buffer, optionally a histidine HCl buffer.
IX. The liquid pharmaceutical composition according to any one of the preceding embodiments, wherein the buffering agent is at a concentration of about 15 to 25 mM.
X. The liquid pharmaceutical composition according to any one of the preceding embodiments, wherein the buffering agent is at a concentration of about 20 mM.
XI. The liquid pharmaceutical composition according to any one of the preceding embodiments, wherein the buffering agent provides a pH of about 5.2 to about 5.8.
XII. The liquid pharmaceutical composition according to any one of the preceding embodiments, wherein the tonicity agent is selected from the group of salts, sugars, and amino acids.
XIII. The liquid pharmaceutical composition according to embodiment XII, wherein the tonicity agent is either sucrose or sodium chloride.
XIV. The liquid pharmaceutical composition according to embodiment XIII, wherein the tonicity agent is sucrose at a concentration of about 200 mM or higher.
XV. The liquid pharmaceutical composition according to embodiment XIII or XIV, wherein the tonicity agent is sucrose at a concentration of about 200 mM-280 mM.
XVI. The liquid pharmaceutical composition according to any one of embodiments XIII to XV, wherein the tonicity agent is sucrose at a concentration of about 240 mM.
XVII. The liquid pharmaceutical composition according to any one of the preceding embodiments, wherein the methionine is at a concentration of about 5-15 mM.
XVIII. The liquid pharmaceutical composition according to embodiment XVII, wherein the methionine is at a concentration of about 10 mM.
XIX. The liquid pharmaceutical composition according to any one of the preceding embodiments, wherein the surfactant is at a concentration of about 0.2-0.8 mg/ml.
XX. The liquid pharmaceutical composition according to any one of the preceding embodiments, wherein the surfactant is polysorbate 20 or poloxamer 188.
XXI. The liquid pharmaceutical composition according to embodiment XX, wherein the surfactant is polysorbate 20 at a concentration of 0.2-0.8 mg/ml.
XXII. The liquid pharmaceutical composition according to embodiment XXI, wherein the surfactant is polysorbate 20 at a concentration of about 0.5 mg/ml.
XXIII. The liquid pharmaceutical composition according to any one of the preceding embodiments, which comprises:
   about 1 to 5 mg/ml of the anti-CD20/anti-CD3 bispecific antibody;
   about 15-25 mM of a histidine buffer;
   about 200-280 mM sucrose;
   about 0-15 mM methionine; and
   about 0.2-0.8 mg/ml of PS20
   at a pH of about 5 to about 6.
XXIV. The liquid pharmaceutical composition according to any one of the preceding embodiments, which comprises:
   about 1 mg/ml of glofitamab;
   about 20 mM of a histidine buffer;
   about 240 mM sucrose;
   about 10 mM methionine; and
   about 0.5 mg/ml of PS20
   at a pH of about 5.5.
XXV. Use of a liquid pharmaceutical composition according to any one of the preceding embodiments for the preparation of a medicament useful for treating a cell proliferative disorder.
XXVI. The pharmaceutical composition according to any one of embodiments I to XXIV for use in treating or delaying progression of a cell proliferative disorder in a subject in need thereof.
XXVII. A method of treating or delaying the progression of a cell proliferative disorder in a subject in need thereof, the method comprising administering to the subject an effective amount of the pharmaceutical composition according to any one of embodiments I to XXIV.
XXVIII. The use, liquid pharmaceutical composition for use, or method according to any one of embodiments XXV to XXVII, wherein the cell proliferative disorder is a cancer.
XXIX. The invention as described hereinbefore.

### EXAMPLES

The following are examples of methods and compositions of the invention. It is understood that various other embodiments may be practiced, given the general description provided above.

### Example 1: Glofitamab in Silico Analysis

RO7082859/Glofitamab is a T-cell bispecific humanized monoclonal antibody (TCB) that binds to human CD20 on tumor cells and to the human CD3 epsilon subunit (CD3s) of the T cell receptor complex (TCR) on T cells. It is comprised of two different heavy chains and two different light chains. Point mutations in the CH3 domain (“Knobs-into-holes”) promote the assembly of two different heavy chains. Exchange of the VH and VL domains in the CD3 binding Fab (“CrossMab approach”) and point mutations in the CH and CL domains (“charged variants”) in the CD20 binding Fabs promote the correct assembly of the two different light chains with the corresponding heavy chains. The “Knobs-into-holes” mutations consist of amino exchanges Y349C, T366S, L368A and Y407V in the heavy chain HC1 and of amino exchanges S354C and T366W in the heavy chain HC2 (Kabat EU index numbering). The “charged variants” mutations consist of amino acid exchanges E123R and Q124K in the light chain LC2 (Kabat numbering) and K147E and K213E in the heavy chains HC1 and HC2 (Kabat EU index numbering).

The binding to human CD20 occurs with high affinity and in a bivalent binding mode, whereas the binding to CD3 s is monovalent and of low affinity. RO7082859 is a human IgG1 with the Fc region bearing a modification (“PG LALA” mutation) which abrogates its binding in vitro to Fc gamma receptors (FcγR), and prevents FcγR-mediated co-activation of innate immune effector cells, including natural killer (NK) cells, monocytes/macrophages and neutrophils without changes in functional binding to FcRn (neonatal Fc receptor). The “PG LALA” mutations consist of amino acid exchanges P329G, L234A, and L235A in the heavy chain HCl and in the heavy chain HC2 (“PG LALA”, Kabat EU index numbering).

The recombinant antibody is produced in CHO cells and consists of two heavy chains (449 and 674 amino acid residues, respectively) and three light chains (232 and 219 (two copies) amino acid residues, respectively), arranged in an asymmetric configuration as illustrated in FIG. 2.

Summary Active Hot Spots

For the CD3 binding moiety of the molecule, in silico prediction indicated two degradation prone Asn residues and one exposed Trp residue in CDR3 of the heavy chain. In a stress experiment over 14 days, no major change in target binding activity was observed after incubation at pH 6.0 but a strong loss of target binding activity was observed after incubation at physiological pH (PBS pH 7.4, data not shown).

### Example 2: Glofitamab Formulation Development GLP Tox and Entry into Human Study

The screen was performed according to the scheme displayed in Table 4. During the screen, the formulations were exposed to the following conditions: 3 and 6-week storage (at 5° C., 25° C. and 40° C.), shaking at 5° C. and 25° C. for 1 week and freeze/thaw (F/T) stress (5 cycles). The nominated formulation is then followed up to 52 weeks.

TABLE 4
Adapted platform screen study design with formulation codes

| | Glofitamab | | | | | |
|---|---|---|---|---|---|---|
| | Protein | | | | | |
| | conc. | | | | | |
| Formulation | (mg/ml) | Buffer | pH | Excipient 1 | Excipient 2 | Surfactant |
| F1 | 5 | 20 mM | 5.5 | 240 mM | 10 mM | 0.05 (w/v)% |
| | | His/His-Cl | | Sucrose | Methionine | PS20 |
| F2 | 5 | 20 mM | 5.5 | 240 mM | — | 0.05 (w/v)% |
| | | His/His-Cl | | Sucrose | | PS20 |
| F3 | 5 | 20 mM | 5.5 | 240 mM | 10 mM | 0.05 (w/v)% |
| | | His/His-Cl | | Sucrose | Methionine | Px188 |
| F4 | 5 | 20 mM | 5.5 | 240 mM | — | 0.05 (w/v)% |
| | | His/His-Cl | | Sucrose | | Px188 |
| F5 | 5 | 20 mM | 6.0 | 240 mM | 10 mM | 0.05 (w/v)% |
| | | His/His-Cl | | Sucrose | Methionine | PS20 |

After 6 weeks of storage at 5° C., 25° C., and 40° C., all formulations remained without significant changes in most of the tested physical properties, namely visible and sub-visible particles, color, turbidity, pH, and protein content. CE-SDS (capillary electrophoresis sodium dodecyl sulfate) data is not shown as it was not critical for the nomination.

Visible particle analysis by the Seidenader method demonstrated no formation of visible particles for either of the formulation at all storage conditions. Subvisible particle count was low (not shown). Under mechanical stress conditions, F2-F5 showed many particles at both 5 and 25° C. F1 was free of particles in both conditions. Using EP and Optima, all compositions were practically free of particles (0 particles) apart from F3 and F4 (both with P188) show particles but below the limit (not shown). Sub-visible particles were significantly worse in F3 (P188+Met) than in F4 (P188) at 5° C. shake, all other formulations have similar counts at each condition (not shown).

Turbidity and color showed no significant changes in all formulations under all conditions after 6 weeks. Surfactant contents were stable at 5 and 25° C., and for both P188 containing formulations (F3, F4) also at 40° C. For all PS20 containing active formulations (F1, F2, and F5), a loss in surfactant content was observed at 40° C., regardless of whether the formulation contained methionine or not.

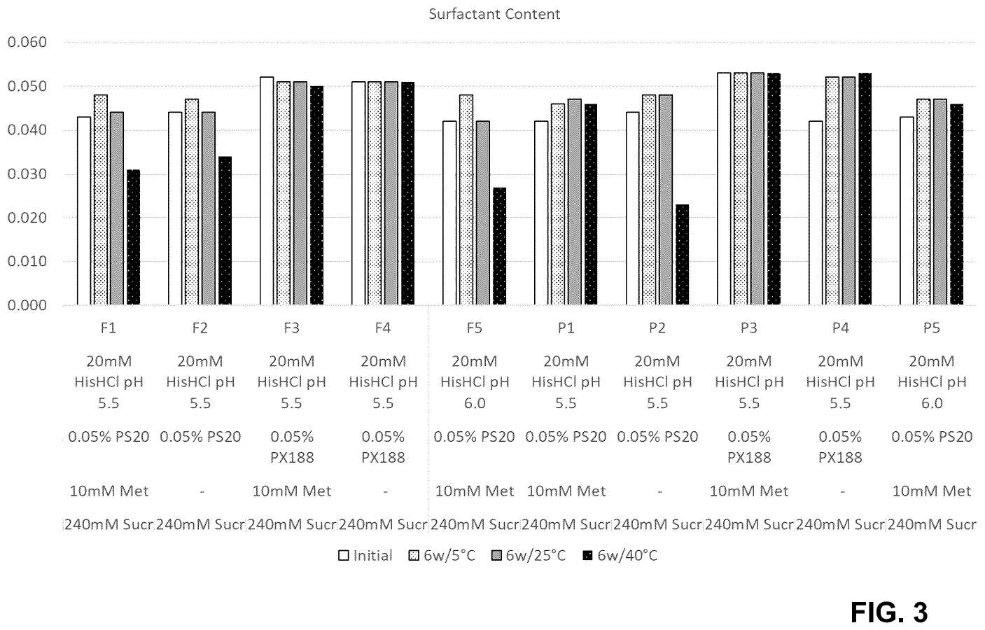

A beneficial effect of methionine could only be seen in the PS20 containing placebo formulations, where only P2 dropped in PS content at 40° C. (FIG. 3). Biochemical characterization revealed differences in formulations only after storage at 40° C.

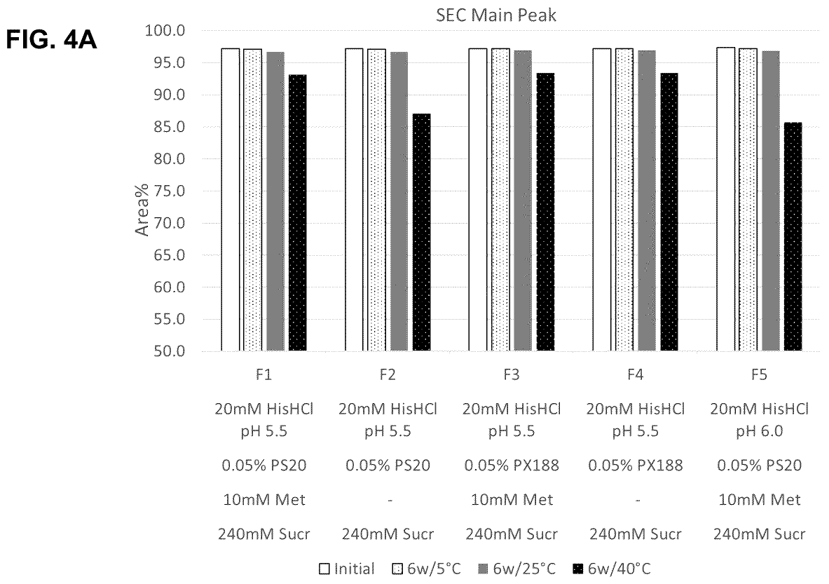

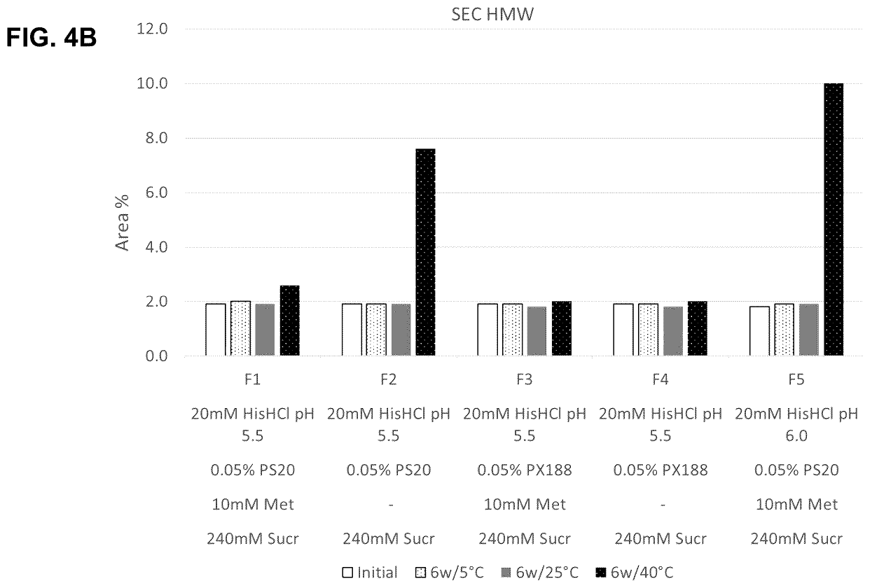

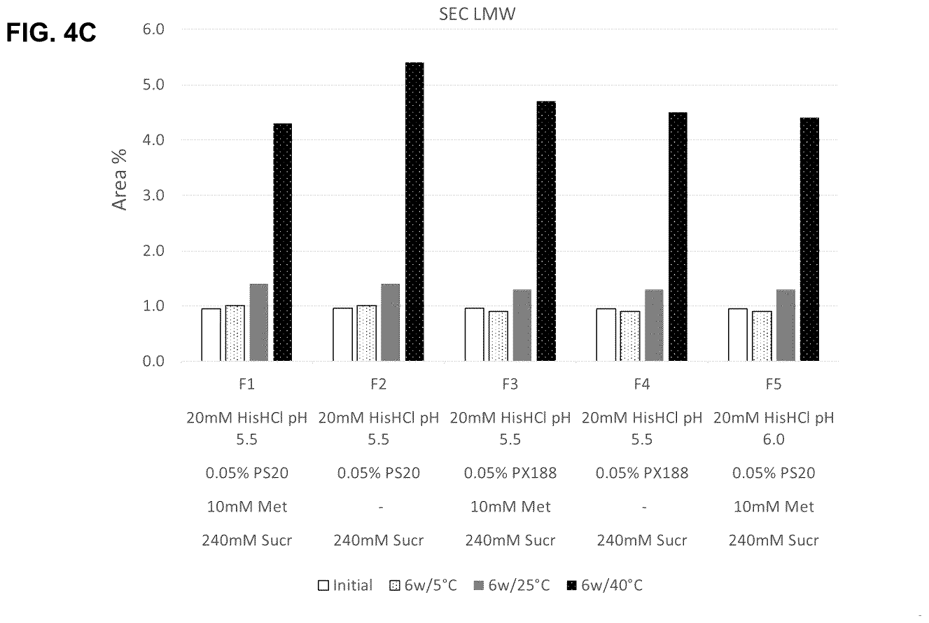

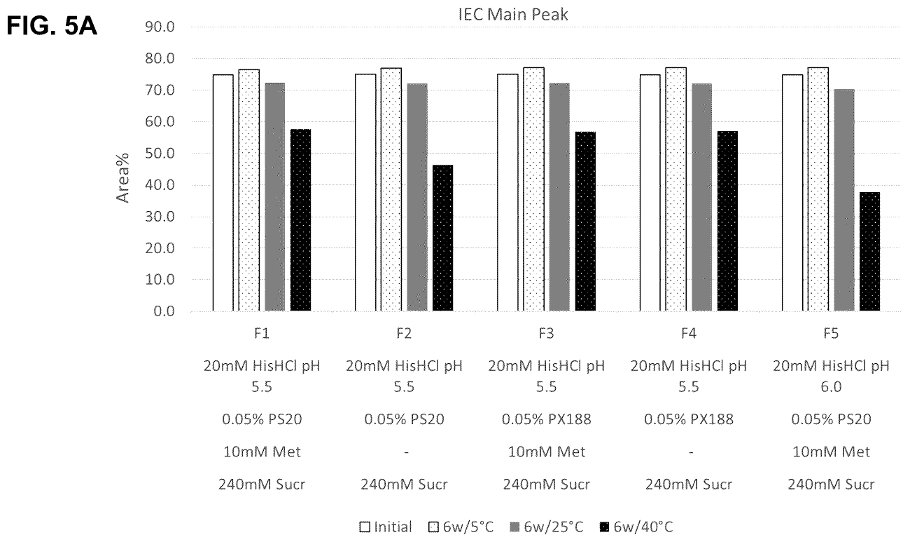

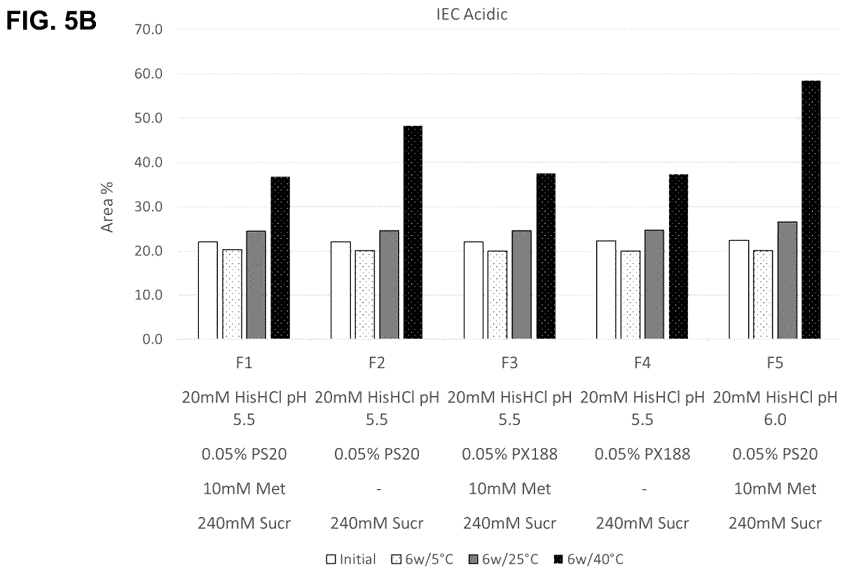

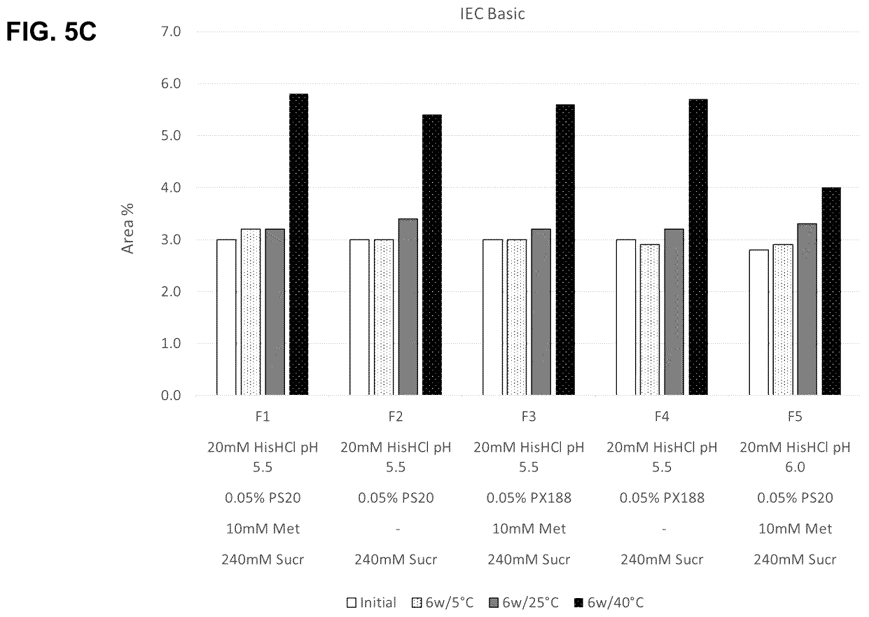

In size exclusion chromatography (SEC), the loss of monomer was more pronounced for F2 and F5, correlating with an increase in HMW (high molecular weight) area. A new HMWspecies could be seen to emerge, only minor in F3 and F4, stronger in F1, and dominant in F2 and F5. LMW (low molecular weight) species were found to increase in all formulations at approximately the same rate (FIG. 4). The similar trend could be observed in ion exchange chromatography (IEC), with an overall increase in Basic peak area, and with the increase of Acidic area being more pronounced in F2 and F5 (FIG. 5).

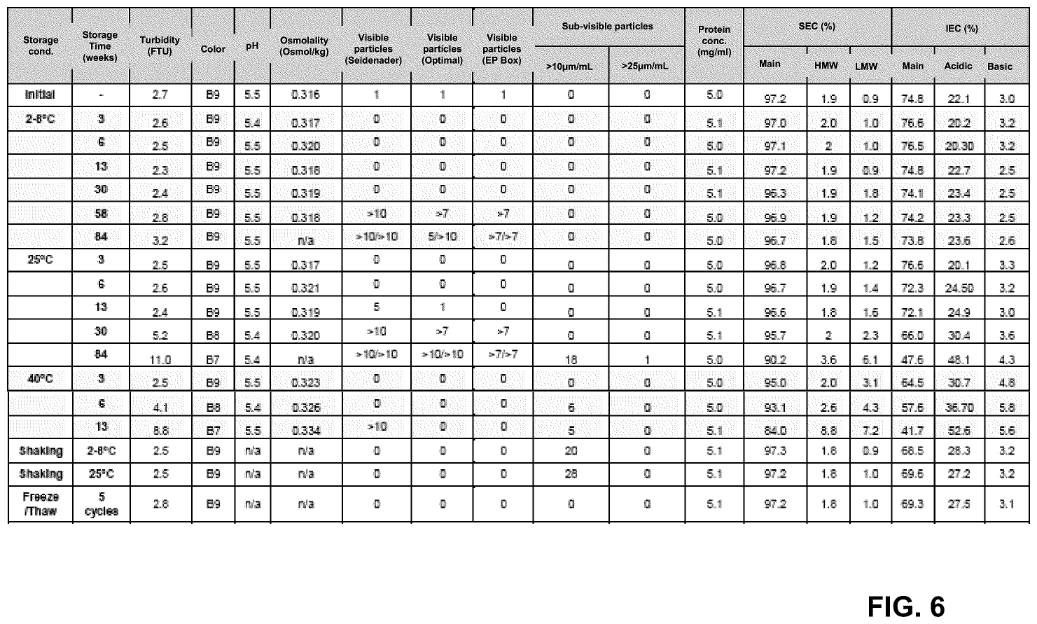

In summary, the data clearly ruled out F2 and F5, and showed F1, F3, and F4 to be equally stable, with no clear preference for any of the three. F1 (5 mg/ml glofitamab, 20 mM Histidine/Histidine HCl, pH 5.5, 240 mM Sucrose, 10 mM Methionine, 0.05% (w/v) PS20) was nominated. A summary of all analytical results for F1 can be found in FIG. 6.

### Example 3: GLP Tox/Entry into Human Study

Binding by BIACORE®

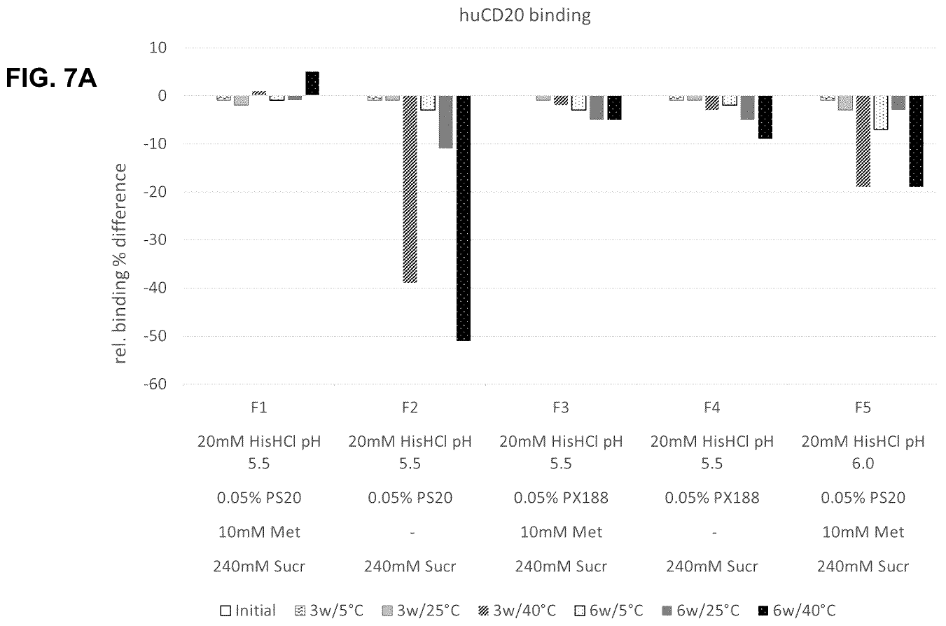

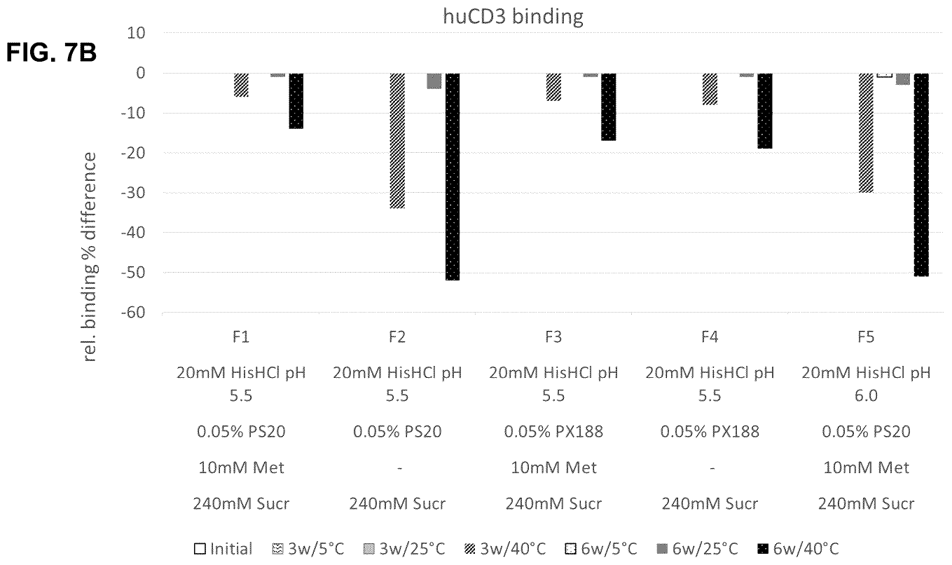

The aforementioned purity results are also reflected in a loss of CD20 binding at 40° C. for F2 and F5, and a strong loss of CD3 binding in those formulations of up to 50%, compared to a loss between 10 to 20% for the remaining formulations (FIG. 7A and FIG. 7B).

### Example 4: Development Studies for Phase III and Commercial Formulation

This Example provides an overview of the pharmaceutical development of the glofitamab formulation. As a result of this development, glofitamab drug product is provided as a sterile liquid concentrate for solution for IV infusion. The drug product is composed of 1 mg/ml glofitamab in 20 mM L-histidine/L-histidine hydrochloride (HCl) buffer, 240 mM sucrose, 10 mM L-methionine, 0.5 mg/ml polysorbate 20, pH 5.5. Glofitamab is the only active ingredient in the drug substance and drug product. Formulation development studies established that the dosage form and formulation are suitable for the intended use. The formulation is sufficiently robust to ensure that the drug product is stable during manufacture, storage, transportation, and administration.

Formulations having higher protein concentrations (e.g., 5, 25, or 50 mg/ml glofitamab) were tested, but were not then pursued because of sub-visible and visible particle formation due to PS20 degradation. The release of free fatty acids (lauric and myristic acids) at levels increasing together with protein concentration confirmed the root cause for sub-visible and visible particle formation as being due to hydrolytic PS20 degradation.

A liquid dosage form was selected enabling few handling steps while ensuring product quality during manufacturing and through end of drug product shelf life.

Glofitamab drug product will be commercially available in two strengths provided in two vial configurations: 2.5 mg/vial filled in a 6-ml single-use glass vial and 10 mg/vial filled in a 15-ml single-use glass vial, to match the required clinical doses of 2.5, 10, and 30 mg, while minimizing product wastage. For commercial drug product formulation, the concentration of glofitamab was reduced to 1 mg/ml while keeping the excipient composition unchanged.

Formulation development studies informed the rationale for the selection of the appropriate dosage form, protein concentration, surfactant concentration, buffer species, solution pH, stabilizer, tonicity agent, and vial configuration for the drug product. The drug substance formulation was optimized to account for facility fit, dilution, and storage considerations.

Selection of Dosage Form

A liquid dosage form was selected to provide a concentrate for solution for infusion requiring few handling steps while ensuring product quality during manufacturing and through end of drug product shelf life.

Selection of Protein Concentration

A protein concentration of 5 mg/ml was selected for the phase I and retained until phase III. A protein concentration of 1 mg/ml was subsequently selected as commercial formulation based on formulation development studies and updated clinical dosing requirements.

The stability of formulations containing 20 mM L-histidine/L-histidine hydrochloride, 10 mM L-methionine, 240 mM D-sucrose and 0.5 mg/ml polysorbate 20 (PS20), at pH 5.5, was tested at glofitamab concentrations of 1 mg/ml, 5 mg/ml, and 25 mg/ml in order to be prepared to adapt the protein concentration to clinical needs. These formulations were evaluated at the initial time point (TO), at several intermediate time points and at the end of the study after 104 weeks of storage at 2° C.-8° C. by assessing purity of glofitamab by SE-HPLC and IE-HPLC, PS20 content and visible/subvisible particle formation.

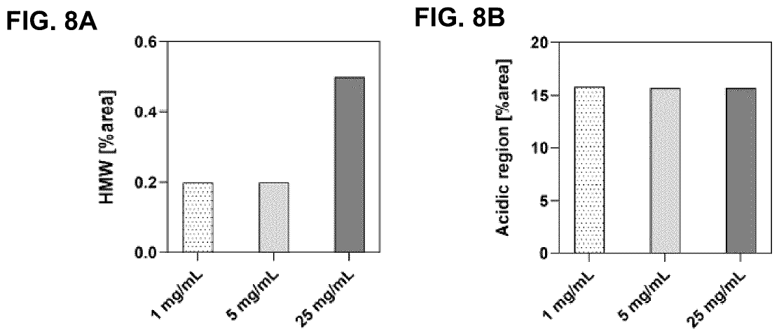

Purity by SE-HPLC and IE-HPLC were comparable between the 1 mg/ml and 5 mg/ml formulations throughout the study (FIG. 8A and FIG. 8B). The subvisible particle counts were also comparable. Furthermore, the 1 mg/ml formulation exhibits no PS20 degradation beyond method variability (FIG. 12, also see below, Assessment of Polysorbate 20 Degradation) compared to the 5 mg/ml and 25 mg/ml formulations. Based on these results and the updated clinical dose regimen of 2.5, 10 and 30 mg, the 1 mg/ml formulation was selected as commercial formulation.

A concentration range of 0.9-1.1 mg/ml protein was further assessed in a subsequent multivariate formulation robustness study (see Example 5, Formulation Robustness Studies). The study confirmed the acceptable stability behavior over this concentration range.

### Selection of PH, Buffer, Stabilizer, and Tonicity Agent

Based on formulation development studies, a 20 mM solution of L-histidine/L-histidine hydrochloride at pH 5.5 was selected as the buffer in combination with 10 mM L-methionine as stabilizer and 240 mM D-sucrose as tonicity agent for the phase I and retained for the phase III and commercial formulation.

A study at 5 mg/ml glofitamab was set up to test a pH range of 5.5 to 6.0 of a 20 mM L-histidine/L-histidine hydrochloride buffer as well as L-methionine levels of 0 and 10 mM. Additionally, a comparison between 240 mM D-sucrose and 130 mM sodium chloride was performed.

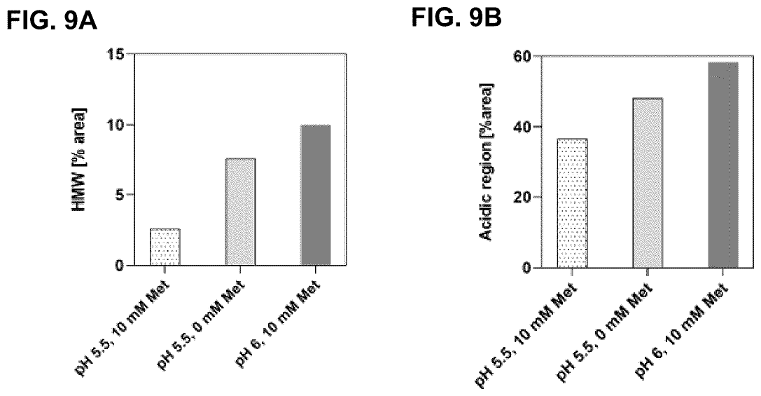

The effect of pH and stabilizer was evaluated at the initial time point (TO) and after 6 weeks of storage at 40° C. by assessing purity of glofitamab by SE-HPLC and IE-HPLC, and visible/subvisible particle formation. The choice of tonicity agent was assessed at the initial time point (TO) and after 26 weeks of storage at 25° C. by measuring SE-HPLC, IE-HPLC, and determine visible/subvisible particle formation. A 20 mM L-histidine/L-histidine hydrochloride buffer at pH 5.5 in combination with 10 mM L-methionine showed lowest formation of high molecular weight species (HMWS) (FIG. 9A) and change in charge variants (FIG. 9B) compared to the corresponding formulation without stabilizer addition or a 20 mM L-histidine/L-histidine hydrochloride buffer/10 mM L-methionine combination at pH 6. A L-histidine/L-histidine hydrochloride monohydrate concentration of 20 mM was shown to be sufficient to maintain the formulation pH during the manufacturing of the drug product as well as during storage of the drug substance and drug product.

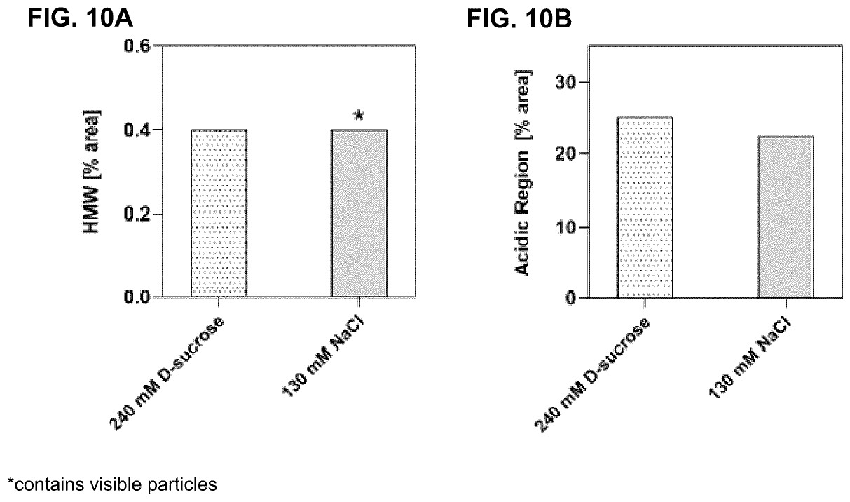

240 mM D-sucrose was chosen based on the comparison between 240 mM D-sucrose and 130 mM sodium chloride. The subvisible particle counts were comparable between the formulations. No visible particle formation was observed after 26 weeks storage at 25° C. for the D-sucrose containing formulation whereas visible particles were observed for the NaCl containing formulation (FIG. 10A and FIG. 10B).

### Selection of Surfactant

PS20, at a concentration of 0.5 mg/ml, was selected for the phase I and retained until commercial formulation based on the results of the stability studies. A study at 50 mg/ml glofitamab in a 20 mM L-histidine/L-histidine hydrochloride buffer, pH 5.5 with 10 mM L-methionine and 240 mM D-sucrose was set up to investigate the stabilizing effect of poloxamer 188 (P188) versus PS20. P188 was tested at levels of 0.5, 0.7, and 1.0 mg/ml; PS20 at levels of 0.1, 0.3, and 0.5 mg/ml.

The effect of the added surfactant was evaluated at the initial time point (TO) and after 7 days of shaking at 25° C. by assessing the purity of glofitamab by SE-HPLC and IE-HPLC, and visible/subvisible particle formation.

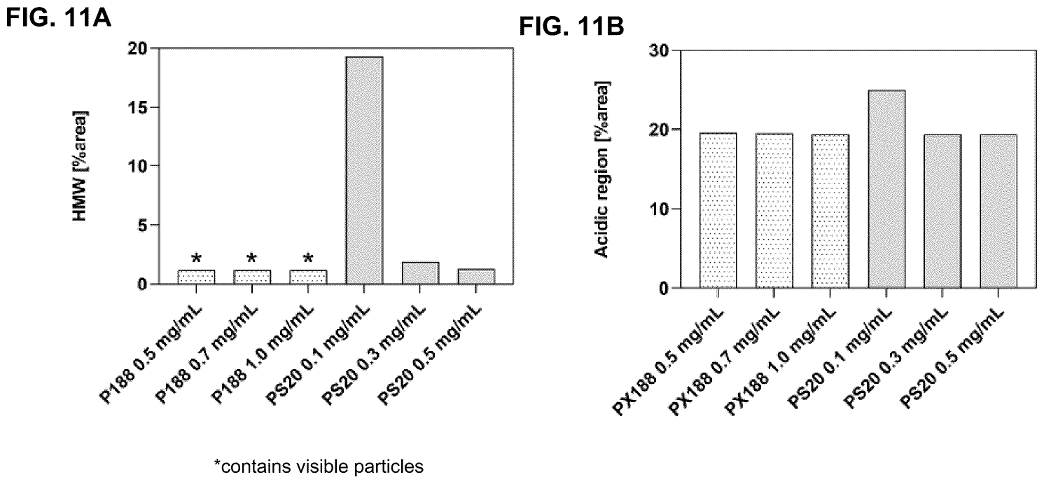

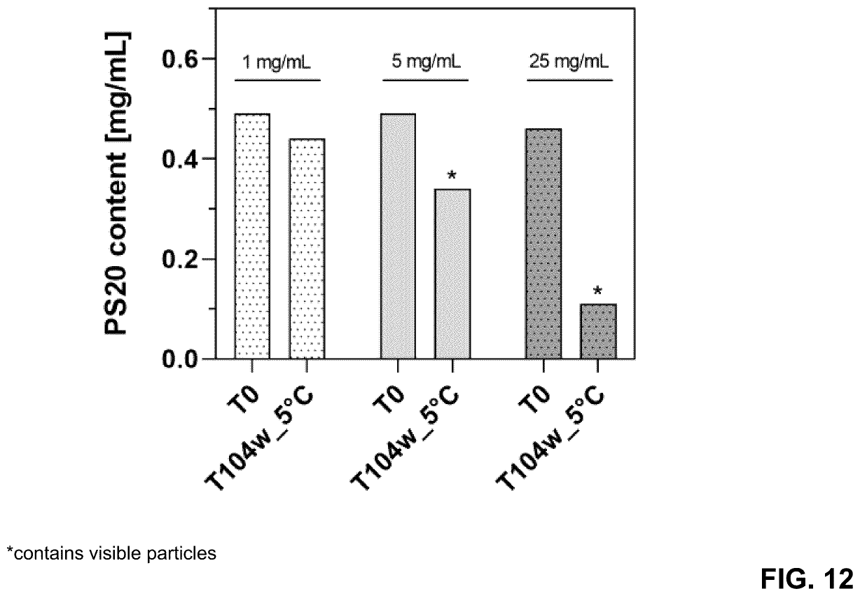

Visible particle formation was observed for all P188 concentrations. It was therefore ruled out as a suitable surfactant for glofitamab (FIG. 11A and FIG. 11B). No visible particles were detected in the PS20 containing formulations after 7 days of shaking at 25° C. (FIG. 11A and FIG. 11B). For the 0.1 mg/ml PS20 containing formulation a substantial increase in HMWS and charge variants was observed, whereas for the 0.3 mg/ml PS20 containing formulation, a slightly increased level of HMWS and charge variants was observed, compared to the 0.5 mg/ml PS20 containing formulation after 7 days of shaking at 25° C. (FIG. 11A and FIG. 11 B). The subvisible particle counts were comparable across the different PS20 concentrations. For the 0.1 mg/ml PS20 containing formulation, a substantial increase in HMWS and charge variants was observed, whereas for the 0.3 mg/ml PS20 containing formulation, a slightly increased level of HMWS and charge variants was observed, compared to the 0.5 mg/ml PS20 containing formulation after 7 days of shaking at 25° C. (FIG. 11A and FIG. 11B). Therefore, the 0.5 mg/ml PS20 containing formulation was selected. A polysorbate 20 level of 0.5 mg/ml was shown to be sufficient to protect glofitamab against stresses that may occur during processing (e.g., agitation, freezing and thawing, or shear stress), handling, storage, and transportation. A concentration range of 0.2-0.8 mg/ml PS20 was further assessed in a subsequent multivariate formulation robustness study (see Example 5, Formulation Robustness Studies). The study confirmed the acceptable stability behavior over this concentration range.

### Example 5: Formulation Robustness Studies

The composition of the drug substance and the drug product can vary within a range based on manufacturing factors such as weighing tolerances of the buffer components. A multivariate formulation robustness study was performed, and it demonstrated that the relevant quality attributes (QAs) of glofitamab are acceptable at the edges of these composition ranges. A multivariate stability study at two levels was conducted on three factors that had been identified as having a potential impact on critical quality attributes (CQAs) during drug product storage. The following three formulation parameters were assessed:
1. Protein concentration
2. pH
3. PS20 concentration
   In addition, three formulation parameters were assessed individually in a univariate stability study:
4. Buffer strength
5. L-methionine concentration
6. D-sucrose concentration

The multivariate formulation robustness study demonstrated that the relevant CQAs of glofitamab are acceptable throughout the entire claimed formulation composition ranges.

### Design of Study

A risk assessment was performed to identify formulation parameters in the drug substance and drug product that are important for maintaining product quality over shelf life. A multivariate study and a univariate study have been set up accordingly.

Multivariate Study (F6 to F12)

A fractional factorial design (resolution III) stability study at two levels was conducted using the three identified formulation parameters protein concentration, pH, and PS20 concentration, as input factors.

Univariate Study (F13 to F20)

L-Methionine and D-sucrose concentration (low and high level), as well as a buffer strength (low and high level) was tested.

One formulation with low protein concentration, low pH and low PS20 concentration was assessed as direct comparison to the corresponding formulation at high pH, high protein concentration and high PS20 concentration.

One formulation with 0.3 mg/ml PS20 concentration was included to support acceptance criteria setting.

The tested formulation parameter ranges are defined to cover either the drug product specification acceptance criteria and/or manufacturing acceptable ranges, as described in Table 5. Table 6 shows the design plan comprising 15 experiments including 3 center points, with the 3 center points corresponding to the target commercial formulation composition.

TABLE 5
Formulation Robustness Study: Target Formulation
and Multivariate and Univariate Study Range

| | | | Lower | Upper |
|---|---|---|---|---|
| | | Target | Level | Level |
| Glofitamab Concentration (mg/ml) | Mab | 1 | 0.9 | 1.1 |
| L-Histidine/L-histidine | His | 20 | 15 | 25 |
| hydrochloride (mM) | | | | |
| pH | pH | 5.5 | 5.0 | 6.0 |
| PS20 Concentration (mg/ml) | PS20 | 0.5 | 0.2 (0.3) | 0.8 |
| D-sucrose Concentration (mM) | Suc | 240 | 200 | 280 |
| L-Methionine Concentration (mM) | Met | 10 | 5 | 15 |

TABLE 6
Formulation Robustness Study Design Plan: Evaluated Glofitamab Formulations

| Multivariate Study | | | | Tested in the Univariate Study | | |
|---|---|---|---|---|---|---|
| | Protein | | PS20 | Buffer | D-Sucrose | L-Methionine |
| | Concentration | | Concentration | Strength | Concentration | Concentration |
| Formulation | (mg/ml) | pH | (mg/ml) | (mM) | (mM) | (mM) |
| F6 | 0.90 | 5.0 | 0.80 | | | |
| F7 | 1.10 | 5.0 | 0.20 | | | |
| F8 | 0.90 | 6.0 | 0.20 | | | |
| F9 | 1.10 | 6.0 | 0.80 | 20 | 240 | 10 |
| F10 (target) | 1.00 | 5.5 | 0.50 | | | |
| F11 (target) | 1.00 | 5.5 | 0.50 | | | |
| F12 (target) | 1.00 | 5.5 | 0.50 | | | |
| Univariate Study | | | | | | |
| F13 | 0.90 | 5.0 | 0.20 | 20 | 240 | 10 |
| F14 | 1.00 | 5.5 | 0.50 | 20 | 200 | 10 |
| F15 | 1.00 | 5.5 | 0.50 | 20 | 280 | 10 |
| F16 | 1.00 | 5.5 | 0.50 | 20 | 240 | 5 |
| F17 | 1.00 | 5.5 | 0.50 | 20 | 240 | 15 |
| F18 | 1.00 | 5.5 | 0.50 | 15 | 240 | 10 |
| F19 | 1.00 | 5.5 | 0.50 | 25 | 240 | 10 |
| F20 | 1.00 | 5.5 | 0.30 | 20 | 240 | 10 |

The stability of glofitamab in the formulation compositions described in Table 6 was evaluated as:
Stability study:
   Storage conditions: Real time (2° C.-8° C.), and accelerated (25° C.)
   Testing frequency: 0, 4, 13, 26 (end of 25° C. storage), 39, 52, 78 and 104 weeks of storage at the above storage conditions
Stress tests:
   5 freeze-thaw cycles,
   Shaking for one week at 2-8° C. and shaking for one week at 25° C.
Stability to support DS: storage at −40° C. for 0, 26, 52 and 104 weeks

### Assessed QAs:

HMWS (high weight molecular species) and Main Peak by SE-HPLC
LMWS (low weight molecular species) and Main Peak by non-reduced CE-SDS,
Acidic Peak 2 and 3, Acidic Region, Basic Region and Main Peak by IE-HPLC
Protein content by ultraviolet-visible spectroscopy
Polysorbate 20 content by HPLC-ELSD
L-Methionine and L-histidine concentration by RP-HPLC
Oxidation, and isomerization by peptide mapping (LC-MS)
Potency by bioassay
Visible particles
Subvisible particles
Color, Clarity/Opalescence
pH
Osmolality
Density

### Overall Data Analysis Procedure

Data for all quality attributes were collected over time for each formulation. The relative change of each QA over time was evaluated.

### Multivariate Study:

A simple linear regression is fitted for each quality attribute and for each formulation over time. Thus, a degradation rate for each quality attribute and each formulation is calculated. If not mentioned explicitly, degradation rates are reported as degradation per week. These degradation rates are evaluated as responses in a Design of Experiment (DoE) study and the effect of the three parameters, protein concentration, pH, and PS20 concentration, on these degradations was investigated. If a quality attribute showed no meaningful change compared to target formulation overtime, regression analysis and effect estimates was not performed. For quality attributes that showed a meaningful change over time, a linear regression was used to estimate the main effects of the three factors on the degradation rates. In addition, main effect plots are shown to illustrate these effects graphically.

### Univariate Study:

For the parameters tested in the univariate study, the results after 39 weeks storage at 2° C.-8° C. were evaluated in comparison to the T0 to identify potential changes. If changes were identified, degradation rates are calculated and compared to the degradation of the target formulation in order to estimate the impact of the investigated formulation parameter at the edges. In some cases, the degradation rate per week was transformed to a degradation observed over 104 weeks by multiplying it with a factor of 104. Regression analysis was performed using JMP® software (SAS Institute, Cary, NC, Version 10.0 or higher).

### Stability of Robustness Formulations at Recommended Storage Condition (2° C.-8° C.):

An overview of the evaluation of the relative change in comparison to the target formulation after 39 weeks storage at 2° C.-8° C. is provided in Table 7. Increased acidic variant levels (Acidic Region and Acidic Peak 2 by IE-HPLC) were observed for all formulations formulated at pH 6 (F8, F9, F20). The observed increase in acidic variants is reflected by a corresponding decrease of the IE-HPLC Main Peak in the impacted formulations. No changes were observed for all other CQAs across all other formulations after 39 weeks at 2° C.-8° C. storage. In conclusion, pH was identified as critical formulation parameter. All other formulation parameters, protein content, PS20, L-methionine, and D-sucrose concentration, as well as buffer strengths did not show an impact on the tested CQAs over the investigated range.

### Stability of Robustness Formulations at Accelerated Storage Conditions (25° C.):

Comparable to the 2° C.-8° C. data, increased acidic variant levels due to deamidation (Acidic Region and Acidic Peak 2 by IE-HPLC) were observed for all formulations formulated at pH 6 (F8, F9, F20), which is reflected in a decrease of the IE-HPLC Main Peak in the impacted formulations. Additionally, increased fragmentation levels were observed by an increase in LMWS by CE-SDS for F1 and F2 which are formulated at pH 5. This increase is reflected in a decrease of the CE-SDS Main Peak. No changes were observed for any other CQAs across all other formulations after 26 weeks at 25° C. storage.

In conclusion, the 25° C. data confirmed that pH is a critical formulation parameter. All other formulation parameters did not show an impact on CQAs.

TABLE 7
Relative Change After Storage of 39 Weeks at
2° C.-8° C. of Relevant CQA

| | Relative Change | Description |
|---|---|---|
| | in Comparison | in Comparison |
| | to Target | to Target |
| | Formulation | Formulation |
| Purity by SE-HPLC | | |
| Sum of HMWs | No Change | |
| Main Peak | No Change | |
| Purity by NR-CE-SDS | | |
| Sum of LMWS | No Change | |
| Main Peak | No Change | |
| Purity by IE-HPLC | | |
| Acidic Peak 2 | Increase | Increase for all |
| | | Formulations at pH 6 |
| | | (F8, F9, F20) |
| Acidic Peak 3 | No Change | |
| Acidic Region | Increase | Increase for all |
| | | Formulations at pH 6 |
| | | (F8, F9, F20) |
| Main Peak | Decrease | Decrease for all |
| | | Formulations at pH 6 |
| | | (F8, F9, F20) |
| Protein Concentration | No Change | |
| Polysorbate 20 Concentration | No Change | |
| L-Methionine and L-Histidine | NA | |
| Concentration by RP-HPLC^{a} | | |
| Tryptophan and Methionine | No Change | |
| Oxidation | | |
| Aspartic Acid Isomerization^{b} | No Change | |
| Potency by Bioassay | No Change | |
| Visible Particles | No Change | |
| Subvisible Particles | No Change | |
| Color, Clarity/Opalescence | No Change | |
| Solution pH | No Change | |
| Osmolality^{a} | NA | |

^{a}Measurement at t = 0 and at the end of the study after 104 weeks.
^{b}Measurement at t = 0 and after 52 and 104 weeks of storage at 2° C.-8° C. only.

### Stability of Robustness Formulations at Recommended Drug Substance Storage Condition (−40° C.):

To support the stability of the drug substance over the entire claimed formulation composition ranges, a stability study of drug product robustness formulations stored at −40° C. was performed. The study results confirmed that no significant change in the tested quality attributes was observed when formulations were stored at the recommended drug substance storage condition of −40° C. for 26 weeks.

Stability of Robustness Formulations after Shaking and Freeze/Thaw Stress:

Formulations were subjected to one week of shaking at 2° C.-8° C. or 25° C. Additionally, the formulations were evaluated after undergoing five freeze/thaw cycles between −40° C. and 5° C. All samples were practically free of visible particles upon shaking or freeze/thaw stress.

Subvisible particles did not change upon shaking and freeze/thaw stress for all formulations. The formulations with low PS20 content (0.2 mg/ml, F7, F8, F13), did not show any product quality impact after shaking and freeze/thaw stress compared to all other formulations containing levels of 0.3-0.8 mg/ml of PS20.

This result confirms that a level of ≥0.2 mg/ml of polysorbate 20 is sufficient to protect the protein against shaking and freeze/thaw stress. Comparably, the formulation with low D-sucrose content (200 mM, F19) did not show any product quality impact after shaking and freeze/thaw stress compared to all other formulations containing levels of 240-280 mM of D-sucrose. This result confirms that a level of ≥200 mM D-sucrose is sufficient to protect the protein against freeze/thaw stress. No substantial changes to any other quality attributes were observed upon shaking or freeze/thaw stress when compared to control samples.

### Linear Regression Analysis of Identified CQAs Based on Data at Recommended Storage Conditions (2° C.-8° C.):

A simple linear regression analysis was performed for the impacted CQAs: solution pH, protein concentration and PS20 concentration. pH was identified to have the main impact. The calculated degradation rates per week were extrapolated to end of shelf-life (EoS) by multiplication with 104 weeks (=24 months). The extrapolated results are summarized in Table 8.

Linear regression analysis demonstrated that there is no meaningful impact of the tested pH range on the identified CQAs, because all CQAs are within the stability acceptance criteria. However, in order to control the increase in Acidic Region, the pH acceptance criterion at drug product release was tightened to 5.2-5.8.

TABLE 8
Results of Linear Regression Analysis Based on 2° C.-8° C. Data

| | pH 5^{a} | | pH 6^{a} | | Target (pH 5.5) | Target |
|---|---|---|---|---|---|---|
| | Extrapolated | pH 5^{a} | Extrapolated | pH 6^{a} | Extrapolated | (pH 5.5) |
| | Degradation | Calculated | Degradation | Calculated | Degradation | calculated |
| | Rate after | Value at | Rate after | Value at | Rate after | value at |
| CQA | 104 weeks | EoS^{b} | 104 weeks | EoS^{b} | 104 weeks | EoS^{b} |
| Acidic Peak | 0.298 | 5.9 | 1.829 | 7.4 | 1.064 | 6.7 |
| 2 (area %) | | | | | | |
| Acidic Peak | 0.147 | 2.8 | 0.481 | 3.1 | 0.314 | 2.9 |
| 3 (area %) | | | | | | |
| Acidic | 1.509 | 15.7 | 3.996 | 18.2 | 2.753 | 16.9 |
| Region | | | | | | |
| (area %) | | | | | | |
| LMWS | 0.574 | 2.4 | 0.134 | 2.0 | 0.354 | 2.2 |
| (area %) | | | | | | |

^{a}All other parameters are set to target for the linear regression analysis.
^{b}Calculated by t = 0 + degradation rate after 104 weeks (average t = 0 over all formulations was used).

### Conclusion:

The extrapolated data suggest an impact of a high pH of 6.0 on the level of acidic variants after 24 months (claimed drug product shelf life). Therefore, the pH acceptance criterion at drug product release was tightened to 5.2-5.8 to limit the formation of acidic forms during drug product stability.

The formulation is considered robust until the end of shelf life since:
CQAs meet the release acceptance criteria at t=0 and after 9 months of storage at 2° C.-8° C. for all formulations at the edges of the formulation ranges
CQAs meet the stability acceptance criteria when using degradation rates for extrapolation to EoS for all formulations at the edges of the formulation ranges.

### Example 6: Assessment of Polysorbate 20 Degradation

Polysorbate 20 can degrade via oxidative or hydrolytic mechanisms. Hydrolytic degradation of polysorbate 20 results in the formation of free fatty acids (FFAs), such as lauric acid. At certain high concentrations, the FFAs may form subvisible or visible particles. Moreover, polysorbate 20 degradation is also a concern if this leads to less polysorbate in the formulation than what is necessary to protect the protein from agitation stress.

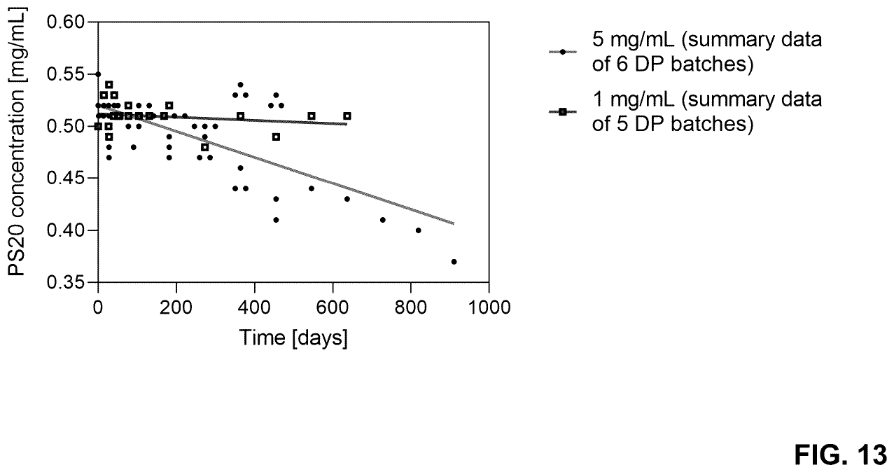

Due to these concerns, polysorbate 20 degradation was monitored during formulation development. PS20 degradation was observed in glofitamab formulations during formulation development with dependence on the protein concentration. Significant PS20 degradation was observed for the 25 mg/ml formulation (FIG. 12) with observation of visible particles at 2° C.-8° C. For the 5 mg/ml formulation, PS20 degradation was less prominent, with observation of visible particles after 20 months. The subvisible particle counts were not impacted. Visible particles were isolated and characterized by fourier transform infrared (FTIR) analysis and were found to be FFA. The 1 mg/ml formulation showed no PS20 degradation (beyond method precision) with absence of visible particle formation throughout the study time of 24 months. The subvisible particle counts were consistently low. Long-term stability data of nine drug product (DP) batches which were derived from four different drug substance (DS) batches confirmed the absence of visible particle. FIG. 13 provides a visualization of long-term stability data of example DP batches.

### Example 7: Physicochemical In-Use Stability Study

Glofitamab drug product is provided as a sterile liquid concentrate for solution for IV infusion. The drug product is composed of 1 mg/ml glofitamab in 20 mM L-histidine/L-histidine hydrochloride buffer, 240 mM sucrose, 10 mM L-methionine, 0.5 mg/ml polysorbate 20, pH 5.5. Glofitamab is a preservative-free drug product supplied in single-dose 2.5-ml and 10-ml glass vials. Glofitamab is intended for IV administration after dilution in 0.9% or 0.45% sodium chloride via IV bag infusion. The proposed registration dose and schedule based on the step-up dosing schedule is 2.5/10/30 mg. The doses are enabled in the IV bag by dose solution concentrations from 0.05 mg/ml to 0.6 mg/ml. In a bracketing approach, 0.05 mg/ml, 0.1 mg/ml and 0.6 mg/ml dose solutions were tested for compatibility to cover the full dose range (Table 9).

Stability and compatibility studies were conducted to confirm the physicochemical stability of the solutions for infusion under recommended in-use conditions. The studies demonstrated that the glofitamab solutions for infusion are stable during typical preparation and administration procedures and may be held for 72 hours at 2° C.-8° C. and an additional 24 hours at 30° C. at ambient room light conditions followed by an infusion at 25° C. taking no longer than 16 hours. The nominal protein concentration range over which the solutions for infusion are demonstrated to be stable is 0.05 to 0.6 mg/ml.

### Study Materials and Setup:

The physicochemical stability of glofitamab was evaluated after dilution into 100 ml or 250 ml IV bags containing 0.9% sodium chloride solution and 0.45% sodium chloride solution, mimicking the handling procedures to be used in the commercial setting. For each diluent, the product quality of glofitamab was evaluated at diluted concentrations of approximately 0.05 mg/ml (low dose, tested in 0.9% sodium chloride only), 0.1 mg/ml (low dose) and 0.6 mg/ml (high dose), which bracket the expected concentration range of the product as outlined in Table 9.

For 0.9% sodium chloride, two different bag types with drug product contact surfaces made of polyvinylchloride (PVC) or polyolefin-polyethylene-polypropylene (PO-PE-PP) were tested. For 0.45% sodium chloride bags with drug product contact surfaces made of PVC were tested. For each diluent, three drug product batches were set up in a matrix approach for this stability assessment. The drug product batches had been stored for 20 months, or 7 months at 2° C.-8° C.

TABLE 9
Simulated In-Use Study Setup (0.9% Sodium Chloride Solution in PVC or
PO-PE-PP IV Bags and 0.45% Sodium Chloride Solution in PVC IV Bags)

| | | Nominal | | | | | |
|---|---|---|---|---|---|---|---|
| | | Protein | 0.9% NaCl/ | Drug | | | |
| | | Concentration | 0.45% NaCl | Product | | | |
| | | in the Bag after | Removed | Injected | | Infusion | Infusion |
| | Dose | Dilution | from Bag | into Bag | Hold Time | Volume | Speed |
| Low | 2.5 mg | 0.05 mg/ml | 12.5 ml* | 12.5 ml | 2° C.-8° C.: 72 h | 250 ml | 0.3 mg/h |
| Dose | | | | | 30° C.: 24 h | | 6 ml/h |
| Low | 2.5 mg | 0.1 mg/ml | 10 ml | 10 ml | 2°° C.-8° C.: 72 h | 100 ml | 0.3 mg/h |
| Dose | | | | | 30° C.: 24 h | | 3 ml/h |
| High | 30 mg | 0.6 mg/ml | 60 ml | 60 ml | 2° C.-8° C.: 72 h | 100 ml | 0.72 mg/h |
| Dose | | | | | 30° C.: 24 h | | 1.2 ml/h |

*tested in 0.9% NaCl only

Infusion of the dosing solution was simulated by passing the diluted glofitamab solutions through the following:
1. Infusion sets with product-contacting surfaces of PVC, polyethylene (PE), polybutadiene (PBD), polyurethane (PUR), silicone, and acrylonitrile butadiene styrene (ABS) with/without 0.2 μm in-line filters made of polysulfone or polyethersulfone (PES).
2. A three-way stopcock infusion aid made from polycarbonate (PC).
3. Catheters made from polyetherurethane (PEU), or polytetrafluoroethylene (PTFE)

The simulated infusion was performed over a period of 16 hours, which is longer than the intended infusion duration of 4-8 hours to ensure compatibility of the dosing solution during extended contact with the materials of construction of the infusion sets and aids.

Samples were collected for analysis from each IV bag after dilution and after the cumulative hold time, as well as at the end of the simulated infusion.

The samples were tested using appropriate stability-indicating methods including purity by SE-HPLC, IE-HPLC and CE-SDS, content of protein by UV, subvisible particles by light obscuration, color, clarity/opalescence, pH, and potency by bioassay. LMW by CE-SDS was measured for high dose (0.6 mg/ml) only, because at a sample concentration of 0.1 mg/ml the signal intensity was too low to allow for meaningful interpretation of the data. However, the product quality was ensured by the presented potency data.

### Results:

The in-use studies demonstrated that glofitamab is physicochemically stable after dilution into 0.9% or 0.45% sodium chloride solution and after holding for 72 hours at 2° C.-8° C. and for an additional 24 hours at 30° C. at ambient room light conditions, followed by simulated infusion at ≤25° C. taking no longer than 16 hours. For 0.5 mg/ml dose solutions, no inline filter should be used.

The drug product batches used in these compatibility studies had previously been stored at the recommended storage temperature (2° C.-8° C.) for 7-20 months, demonstrating that drug product age does not impact stability during in-use handling and administration.

### Example 8: Microbiological Stability

The drug product must be diluted before administration using aseptic technique. Solutions of glofitamab for IV administration are prepared by dilution of the drug product into an infusion bag containing 0.9% sodium chloride or 0.45% sodium chloride. The prepared infusion solution should be used immediately. The drug product does not contain any antimicrobial preservative; therefore, sterility of the solution must be ensured during in-use handling by maintaining appropriate aseptic conditions.

Microbiological challenge studies were performed to evaluate the propensity of the solutions to support microbiological proliferation, in case an accidental contamination was to occur. The proliferation of seven different test microorganisms (listed in USP <51>) at 2° C.-8° C. for up to 96 hours and at 20° C.-25° C. for up to 48 hours was assessed. The results met the acceptance criterion of “no growth,” when a difference of not more than 0.5 log₁₀ unit higher than the initial value was measured.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A liquid pharmaceutical composition comprising:
about 1 to 25 mg/ml of an anti-CD20/anti-CD3 bispecific antibody;
about 10 to 50 mM of a buffering agent;
about ≥200 mM of a tonicity agent;
about 0-15 mM methionine; and
about ≥0.2 mg/ml of a surfactant;
at a pH in the range of from about 5.0 to about 6.0,
wherein the anti-CD20/anti-CD3 bispecific antibody comprises;
a) at least one antigen binding domain that specifically binds to CD20 comprising:
a heavy chain variable region comprising:
(i) an HVR-H1 comprising the amino acid sequence of SEQ ID NO: 1;
(ii) an HVR-H2 comprising the amino acid sequence of SEQ ID NO: 2; and
(iii) an HVR-H3 comprising the amino acid sequence of SEQ ID NO:3;
and a light chain variable region comprising:
(i) an HVR-L1 comprising the amino acid sequence of SEQ ID NO: 4;
(ii) an HVR-L2 comprising the amino acid sequence of SEQ ID NO: 5; and
(iii) an HVR-L3 comprising the amino acid sequence of SEQ ID NO: 6; and
b) at least one antigen binding domain that specifically binds to CD3 comprising:
a heavy chain variable region comprising:
(i) an HVR-H1 comprising the amino acid sequence of SEQ ID NO: 9;
(ii) an HVR-H2 comprising the amino acid sequence of SEQ ID NO: 10; and
(iii) an HVR-H3 comprising the amino acid sequence of SEQ ID NO:11; and
a light chain variable region comprising:
(i) an HVR-L1 comprising the amino acid sequence of SEQ ID NO: 12;
(ii) an HVR-L2 comprising the amino acid sequence of SEQ ID NO: 13; and
(iii) an HVR-L3 comprising the amino acid sequence of SEQ ID NO: 14.

2. The liquid pharmaceutical composition according to claim 1, wherein the anti-CD20/anti-CD3 bispecific antibody concentration is in the range of about 1 to 5 mg/ml.

3. The liquid pharmaceutical composition according to claim 1, wherein the anti-CD20/anti-CD3 bispecific antibody concentration is in the range of about 0.9-1.1 mg/ml.

4. The liquid pharmaceutical composition according to claim 1, wherein the anti-CD20/anti-CD3 bispecific antibody concentration is about 1 mg/ml.

5. The liquid pharmaceutical composition according to claim 1, wherein the anti-CD20/anti-CD3 bispecific antibody comprises;
a) at least one antigen binding domain that specifically binds to CD20 comprising a heavy chain variable region comprising the amino acid sequence of SEQ ID NO: 7 and a light chain variable region comprising the amino acid sequence of SEQ ID NO: 8, and
b) at least one antigen binding domain that specifically binds to CD3 comprising a heavy chain variable region comprising the amino acid sequence of SEQ ID NO: 15 and a light chain variable region comprising the amino acid sequence of SEQ ID NO: 16.

6. The liquid pharmaceutical composition according to claim 1, wherein the anti-CD20/anti-CD3 bispecific antibody comprises;
a) a first Fab molecule which specifically binds to CD3, wherein the variable domains VL and VH of the Fab light chain and the Fab heavy chain are replaced by each other;
b) a second Fab molecule and a third Fab molecule which each specifically binds to CD20, wherein in the constant domain CL of each of the second Fab molecule and third Fab molecule the amino acid at position 124 is substituted by lysine (K) (numbering according to Kabat) and the amino acid at position 123 is substituted by lysine (K) or arginine (R) (numbering according to Kabat), and wherein in the constant domain CH1 of each of the second Fab and third Fab molecule the amino acid at position 147 is substituted by glutamic acid (E) (EU numbering) and the amino acid at position 213 is substituted by glutamic acid (E) (EU numbering); and
c) a Fc domain composed of a first and a second subunit capable of stable association.

7. The liquid pharmaceutical composition according to claim 1, wherein the anti-CD20/anti-CD3 bispecific antibody is glofitamab.

8. The liquid pharmaceutical composition according to claim 1, wherein the buffering agent:
a) is a histidine buffer;
b) is at a concentration of about 15 to 25 mM; and/or
c) provides a pH of about 5.2 to about 5.8.

9. (canceled)

10. The liquid pharmaceutical composition according to claim 8, wherein the buffering agent is at a concentration of about 20 mM.

11. (canceled)

12. The liquid pharmaceutical composition according to claim 1, wherein the tonicity agent is selected from the group of salts, sugars, and amino acids.

13-14. (canceled)

15. The liquid pharmaceutical composition according to claim 12, wherein the tonicity agent is sucrose at a concentration of about 200 mM-280 mM.

16. The liquid pharmaceutical composition according to claim 12, wherein the tonicity agent is sucrose at a concentration of about 240 mM.

17. The liquid pharmaceutical composition according to claim 1, wherein the methionine is at a concentration of about 5-15 mM.

18. The liquid pharmaceutical composition according to claim 17, wherein the methionine is at a concentration of about 10 mM.

19. The liquid pharmaceutical composition according to claim 1, wherein the surfactant is:
a) at a concentration of about 0.2-0.8 mg/ml; and/or
b) polysorbate 20 or poloxamer 188.

20. (canceled)

21. The liquid pharmaceutical composition according to claim 19, wherein the surfactant is polysorbate 20 at a concentration of 0.2-0.8 mg/ml.

22. The liquid pharmaceutical composition according to claim 21, wherein the surfactant is polysorbate 20 at a concentration of about 0.5 mg/ml.

23. A liquid pharmaceutical composition comprising:
about 1 to 5 mg/ml of the anti-CD20/anti-CD3 bispecific antibody;
about 15-25 mM of a histidine buffer;
about 200-280 mM sucrose;
about 0-15 mM methionine; and
about 0.2-0.8 mg/ml of polysorbate 20
at a pH of about 5 to about 6.

24. A liquid pharmaceutical composition comprising:
about 1 mg/ml of glofitamab;
about 20 mM of a histidine buffer;
about 240 mM sucrose;
about 10 mM methionine; and
about 0.5 mg/ml of polysorbate 20
at a pH of about 5.5.

25. (canceled)

26. The liquid pharmaceutical composition according to claim 19, wherein the molar ratio of the polysorbate 20 to the anti-CD20/anti-CD3 bispecific antibody is between 50 and 100.

27. The liquid pharmaceutical composition according to claim 26, wherein the molar ratio of the polysorbate 20 to the anti-CD20/anti-CD3 bispecific antibody is about 79.

28-29. (canceled)

30. A method of treating or delaying the progression of a cell proliferative disorder in a subject in need thereof, the method comprising administering to the subject an effective amount of the liquid pharmaceutical composition according to claim 1.

31-32. (canceled)


SEQUENCE LISTING (58 sequences)

SEQ ID NO: 1 - protein, 5 aa, synthetic construct
YSWIN

SEQ ID NO: 2 - protein, 17 aa, synthetic construct
RIFPGDGDTDYNGKFKG

SEQ ID NO: 3 - protein, 10 aa, synthetic construct
NVFDGYWLVY

SEQ ID NO: 4 - protein, 16 aa, synthetic construct
RSSKSLLHSNGITYLY

SEQ ID NO: 5 - protein, 7 aa, synthetic construct
QMSNLVS

SEQ ID NO: 6 - protein, 9 aa, synthetic construct
AQNLELPYT

SEQ ID NO: 7 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 9 - protein, 5 aa, synthetic construct
TYAMN

SEQ ID NO: 10 - protein, 19 aa, synthetic construct
RIRSKYNNYATYYADSVKG

SEQ ID NO: 11 - protein, 14 aa, synthetic construct
HGNFGNSYVSWFAY

SEQ ID NO: 12 - protein, 14 aa, synthetic construct
GSSTGAVTTSNYAN

SEQ ID NO: 13 - protein, 7 aa, synthetic construct
GTNKRAP

SEQ ID NO: 14 - protein, 9 aa, synthetic construct
ALWYSNLWV

SEQ ID NO: 15 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 16 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 17 - protein, 672 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 18 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 19 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
GGGGS

SEQ ID NO: 22 - protein, 5 aa, synthetic construct
SGGGG

SEQ ID NO: 23 - protein, 9 aa, synthetic construct
GGGGSGGGG

SEQ ID NO: 24 - protein, 10 aa, synthetic construct
GGGGSGGGGS

SEQ ID NO: 25 - protein, 11 aa, synthetic construct
DGGGGSGGGGS

SEQ ID NO: 26 - protein, 20 aa, synthetic construct
GGGGSGGGGSGGGGSGGGGS

SEQ ID NO: 27 - protein, 12 aa, synthetic construct
GGGSGGGSGGGS

SEQ ID NO: 28 - protein, 16 aa, synthetic construct
GGGSGGGSGGGSGGGS

SEQ ID NO: 29 - protein, 20 aa, synthetic construct
GGGSGGGSGGGSGGGSGGGS

SEQ ID NO: 30 - protein, 24 aa, synthetic construct
GGGSGGGSGGGSGGGSGGGSGGGS

SEQ ID NO: 31 - protein, 10 aa, synthetic construct
GGGGSGGGGS

SEQ ID NO: 32 - protein, 15 aa, synthetic construct
GGGGSGGGGSGGGGS

SEQ ID NO: 33 - protein, 20 aa, synthetic construct
GGGGSGGGGSGGGGSGGGGS

SEQ ID NO: 34 - protein, 25 aa, synthetic construct
GGGGSGGGGSGGGGSGGGGSGGGGS

SEQ ID NO: 35 - protein, 13 aa, synthetic construct
GGGSGGGSGGGSG

SEQ ID NO: 36 - protein, 17 aa, synthetic construct
GGGSGGGSGGGSGGGSG

SEQ ID NO: 37 - protein, 21 aa, synthetic construct
GGGSGGGSGGGSGGGSGGGSG

SEQ ID NO: 38 - protein, 25 aa, synthetic construct
GGGSGGGSGGGSGGGSGGGSGGGSG

SEQ ID NO: 39 - protein, 11 aa, synthetic construct
GGGGSGGGGSG

SEQ ID NO: 40 - protein, 16 aa, synthetic construct
GGGGSGGGGSGGGGSG

SEQ ID NO: 41 - protein, 21 aa, synthetic construct
GGGGSGGGGSGGGGSGGGGSG

SEQ ID NO: 42 - protein, 26 aa, synthetic construct
GGGGSGGGGSGGGGSGGGGSGGGGSG

SEQ ID NO: 43 - protein, 14 aa, synthetic construct
GGGSGGGSGGGSGG

SEQ ID NO: 44 - protein, 18 aa, synthetic construct
GGGSGGGSGGGSGGGSGG

SEQ ID NO: 45 - protein, 22 aa, synthetic construct
GGGSGGGSGGGSGGGSGGGSGG

SEQ ID NO: 46 - protein, 26 aa, synthetic construct
GGGSGGGSGGGSGGGSGGGSGGGSGG

SEQ ID NO: 47 - protein, 12 aa, synthetic construct
GGGGSGGGGSGG

SEQ ID NO: 48 - protein, 17 aa, synthetic construct
GGGGSGGGGSGGGGSGG

SEQ ID NO: 49 - protein, 22 aa, synthetic construct
GGGGSGGGGSGGGGSGGGGSGG

SEQ ID NO: 50 - protein, 27 aa, synthetic construct
GGGGSGGGGSGGGGSGGGGSGGGGSGG

SEQ ID NO: 51 - protein, 15 aa, synthetic construct
GGGSGGGSGGGSGGG

SEQ ID NO: 52 - protein, 19 aa, synthetic construct
GGGSGGGSGGGSGGGSGGG

SEQ ID NO: 53 - protein, 23 aa, synthetic construct
GGGSGGGSGGGSGGGSGGGSGGG

SEQ ID NO: 54 - protein, 27 aa, synthetic construct
GGGSGGGSGGGSGGGSGGGSGGGSGGG

SEQ ID NO: 55 - protein, 13 aa, synthetic construct
GGGGSGGGGSGGG

SEQ ID NO: 56 - protein, 18 aa, synthetic construct
GGGGSGGGGSGGGGSGGG

SEQ ID NO: 57 - protein, 23 aa, synthetic construct
GGGGSGGGGSGGGGSGGGGSGGG

SEQ ID NO: 58 - protein, 28 aa, synthetic construct
GGGGSGGGGSGGGGSGGGGSGGGGSGGG